(19) United States
(12) Patent Application Publication
(10) Pub. No.: US 2021/0046159 A1
(43) Pub. Date: Feb. 18, 2021

(54) IL-1 ANTAGONIST AND TOXICITY INDUCED BY CELL THERAPY

(71) Applicants: OSPEDALE SAN RAFFAELE S.R.L., Milano (Ml) (IT); FONDAZIONE CENTRO SAN RAFFAELE, Milano (IT)

(72) Inventors: Attilio BONDANZA, Milano (IT); Barbara CAMISA, Milano (IT); Margherita NORELLI, Milano (IT)

(73) Assignees: OSPEDALE SAN RAFFAELE S.R.L., Milano (MI) (IT); FONDAZIONE CENTRO SAN RAFFAELE, Milano (IT)

(21) Appl. No.: 16/978,897

(22) PCT Filed: Mar. 8, 2019
(86) PCT No.: PCT/EP2019/055810

§ 371 Date: Sep. 8, 2020

Related U.S. Application Data
(60) Provisional application No. 62/640,920, filed on Mar. 9, 2018.

(51) Int. Cl. A61K 38/20; A61P 39/00

(52) U.S. Cl. CPC A61K 38/2006; A61P 39/00

(57) ABSTRACT

The present invention relates to a IL-1 antagonist alone or in combination with other therapeutic agents and relative pharmaceutical compositions for use for the treatment and/or prevention of toxicity induced by a T cell therapy, wherein the T cell expresses at least one recombinant receptor.

### TECHNICAL FIELD

The present invention relates to a IL-1 antagonist alone or in combination with other therapeutic agents and relative pharmaceutical compositions for use for the treatment and/or prevention of toxicity induced by a T cell therapy, wherein the T cell expresses at least one recombinant receptor.

### BACKGROUND ART

Genetically engineering T cells with chimeric antigen receptors (CARs) represents a highly sophisticated and radically innovative way of treating cancer. The basic structure of CARs comprises a tumor-targeting domain, usually from the single-chain fragment variables (scFvs) of a monoclonal antibody (mAb), fused to at least one immune tyrosine activatory motif (ITAM), typically the CD3 zeta chain, and one or more costimulatory endodomains¹. In pioneering clinical trials, the incorporation of costimulatory endodomains from either CD28²⁻⁴ or 4-1BB^{5,6} into CD19-specific CARs proved to be decisive for engineered T-cell persistence and antitumor effects against chronic lymphocytic leukemia (CLL)^{7,8}, B cell acute lymphoblastic leukemia (ALL)⁹⁻¹² and non-Hodgkin lymphoma (NHL)¹³⁻¹⁵ refractory or relapsed after standard treatments, including bispecific antibodies, allogeneic hematopoietic stem cell transplantation (HSCT) and targeted therapies. More recently, the FDA approval of two distinct CD19 CAR-T cell products in pediatric/young adult ALL and in NHL¹⁶ has paved the way to their availability outside clinical trials. Unfortunately, remarkable antitumor efficacy by CD19 CAR-T cells is accompanied by a number of toxicities, the most obvious being profound and, in some cases, long-lasting B cell aplasia. Instead, the almost invariant development of an early systemic inflammatory syndrome, also known as cytokine release syndrome (CRS), was initially quite unexpected, at least in its severity. Clinical manifestations of CRS typically develop within the first days from CD19 CAR-T cell infusion and include high fever, increased levels of acute phase proteins, respiratory and cardiovascular insufficiency, which if severe and left untreated may lead to death¹⁷. Recognized factors for life-threatening CRS are tumor burden¹⁷ and in vivo peak expansion of CAR-T cells promoted by prior lymphodepletion^{8,12}. CRS responsiveness to the anti-IL-6 receptor (IL-6R) monoclonal antibody (mAb) tocilizumab, as well as correlative biomarker studies^{17,18}, have consolidated a central role for IL-6 signaling in the pathogenesis of this syndrome. A revised grading system has been also proposed, with the aim of precociously identifying patients at high risk for severe CRS and of guiding targeted interventions¹⁹.

Besides CRS, another increasingly reported complication of CD19 CAR-T cells is represented by neurotoxicity. Signs of neurological dysfunction, including headache, confusion, hallucinations, aphasia and seizures, often develop also during CRS, but usually subside after its resolution. Nonetheless, a delayed form of neurotoxicity has been reported to occur days after disappearance of all CRS signs¹⁰⁻¹². Moreover, neurotoxicity by CD19 CAR-T cells is seemingly more frequent in ALL patients and, at odds with initial conjectures, tends to occur independently from CNS localization of leukemia. Since similar neurological events have been also observed with the CD19/CD3 bispecific mAb blinatutomab²⁰, some authors have speculated that neurotoxicity might be, for some reasons, specifically related to the CD19 antigen. Interestingly, although effective in CRS management, preliminary clinical experience suggests that tocilizumab might fail at successfully preventing delayed neurotoxicity.

Widely used preclinical mouse models of CAR-T cell therapy of leukemia rely on xeno-engraftment of primary human acute myeloid leukemia (AML) cells^{21,22} and B-ALL cells²³, or more frequently cell lines²⁴⁻²⁷ in highly immunocompromised non-obese diabetic (NOD)/severe combined immunodeficient/double y-chain knock-out (NSG) mice. Although clearly informative on general fitness and short-term tumor-targeting capacity of CAR-T cells, currently available xenograft mouse models are poorly predictive of long-term antitumor efficacy. The lack of by-stander human hematopoiesis, for example, limits the availability of factors supporting in vivo human T cell persistence and function, requiring in some cases exogenous supplementation²⁸. Moreover, since human engineered T cells retain significant residual xenoreactivity, xenogeneic graft-versus-host disease (X-GVHD) ultimately ensues^{29,30}, thwarting the interpretation of other immune-related toxicities. Different approaches are being studied in order to re-create a microenvironment that better supports human immune functions in immunocompromised mice, including reconstitution of a functional human lympho-hematopoietic system via transplantation of hematopoietic stem cells (HSCs)³¹ and germ-line expression of human cytokines, either by transgenic³² or knock-in means³³. Although these methodologies promise to better model the complex immune interactions that influence antitumor efficacy and toxicities by CAR-T cells, xenoreactivity and resulting X-GVHD remain challenging problems³⁴. To overcome these issues, syngeneic mouse models are increasingly employed and have so far provided useful information on the determinants of B cell aplasia by CD19 CAR-T cells³⁵⁻³⁷ and on the CAR structural cues for avoiding GVHD in case of allogeneic donors³⁸. So far, for reasons that still need to be fully elucidated, both xenograft and syngeneic mouse models have failed to reproduce CRS and neurotoxicity. Moreover, since tocilizumab does not cross-react with mouse IL-6R, the same models cannot be used for a comprehensive assessment of its clinical appropriateness, especially in light of preserved antitumor efficacy.

Various immunotherapy and/or cell therapy methods are available for treating diseases and conditions. Improved methods are needed, for example, to reduce the risk of toxicity of such methods. For example, improved methods are needed to reduce the risk of toxicity to cell therapies, while maintaining exposure of the subject to the administered cells, for example, due to expansion and/or persistence of the administered cells. Provided are methods and uses that meet such needs.

Certain available methods for treating or ameliorating toxicity may not always be entirely satisfactory. Many such approaches focus, for example, on targeting downstream effects of toxicity, such as by cytokine blockade, and/or delivering agents such as high-dose steroids which can also eliminate or impair the function of administered cells. Additionally, such approaches often involve administration of such interventions only upon detection of physical signs or symptoms of toxicity, which in general involve signs or symptoms of moderate or severe toxicity (e.g. moderate or severe CRS or moderate or severe neurotoxicity). Many of these other approaches also do not prevent other forms of toxicity such as neurotoxicity, which can be associated with adoptive cell therapy.

In some cases, this is at a time where such symptoms are severe, and that therefore may require even harsher or more extreme treatments (e.g. higher dosages or an increased frequency of administration) to ameliorate or treat the toxicity.

The use of certain alternative approaches does not provide satisfactory solutions to such issues. In some cases, such agents and therapies (e.g. steroids) are themselves associated with toxic side effects. Such side effects may be even greater at the higher dose or frequency in which is it necessary to administer or treat with the agent or therapy in order to treat or ameliorate the severity of the toxicity that can result from cell therapy. In addition, in some cases, it is believed that an agent or therapy for treating a toxicity may limit the efficacy of the cell therapy, such as the efficacy of the chimeric receptor (e.g. CAR) expressed on cells provided as part of the cell therapy (Sentman (2013) Immunotherapy, 5: 10).

### SUMMARY OF THE INVENTION

In the present invention, the inventors have established a new xenotolerant mouse model recapitulating all toxicities observed with CD19 CAR-T cells in humans, including B cell aplasia, CRS and neurotoxicity, and took advantage of this model to shed light on their mechanisms. The results obtained address fundamental questions to the CAR-T cell field, among others: whether similar toxicities apply to hematological tumor antigens other than CD19, whether their pharmacological prophylaxis or treatment interfere with antileukemia efficacy and whether there are ways for managing neurotoxicity. For comparison with CD19 CAR-T cells, throughout the study the inventors used CAR-T cells specific for CD44v6²¹, an antigen overexpressed on AML and multiple myeloma (MM), as well as on circulating monocytes.

The remarkable antileukemia efficacy by CD19-specific chimeric antigen receptor (CAR) T cells reported so far in humans is frequently associated with life-threatening cytokine release syndrome (CRS) and neurotoxicity. To recapitulate these toxicities and gauge into their pathogenesis, T cells reconstituting in NSG mice transgenic for human stem cell factor (SCF), IL-3 and GM-CSF (SGM3) after transplantation with human hematopoietic stem cells (HSCs) were CAR-engineered ex vivo and infused into secondary recipients co-engrafted with human HSCs and leukemia. Xenogeneic graft-versus-host disease was avoided, and, in case of high leukemia burden, tumor clearance was accompanied by severe CRS, characterized by high fever and elevated systemic human IL-6 levels. CRS lethality was similar between mice infused with CD19 CAR-T cells or CAR-T cells specific for CD44v6, a target antigen expressed on leukemia and monocytes. As demonstrated in vivo by single-cell RNA sequencing and flow cytometry, human monocytes were major sources of IL-1 and IL-6 during CRS. Accordingly, the syndrome was prevented by depleting circulating monocytes or by administering the anti-human IL-6 receptor monoclonal antibody tocilizumab. Despite preservation of antileukemia efficacy, tocilizumab administration failed to protect mice from delayed lethal neurotoxicity, characterized by meningeal inflammation at histopathology. Instead, in the present invention it was surprisingly found that administering an IL-1 receptor antagonist, such as anakinra, abolished both CRS and neurotoxicity, resulting in significant prolongation of survival in the absence of leukemia.

The present disclosure relates to methods for preventing or ameliorating toxicity caused by or due to a cell therapy by pre-emptive or early administration of an IL-1 antagonist. In some embodiments, the therapy is a cell therapy in which the cells generally express recombinant receptors such as chimeric receptors, e.g., chimeric antigen receptors (CARs) or other transgenic receptors such as T cell receptors (TCRs). Features of the methods, including the timing of the administration of the agents or treatments for toxicity, provide various advantages, such as lower toxicity while maintaining persistence and efficacy of the administered cells.

The provided methods offer advantages over available approaches. In some embodiments, the provided methods involve the early or preemptive treatment of subjects prior to the subjects exhibiting physical signs or symptom of toxicity that are more than mild, such as prior to exhibiting physical signs or symptoms of severe toxicity. In some embodiments, the treatment occurs at a time in which a physical sign or symptom of mild toxicity is present, but before moderate or severe toxicity has developed or before extremely severe toxicity has developed. In some embodiments, the treatment occurs at a time in which a physical sign or symptom of mild neurotoxicity, such as grade 1 neurotoxicity is present, but before moderate or severe neurotoxicity has developed or before grade 2 or grade 3 neurotoxicity has developed. In some embodiments, the treatment with the IL-1 antagonist occurs at a time at which no physical signs or symptom of neurotoxicity has developed. Thus, in some cases, the provided methods provide the ability to intervene early before undesired CNS-related outcomes can result. In some cases, the ability to intervene early in the treatment of a toxic outcome or the potential of a toxic outcome.

The present invention provides a IL-1 antagonist for use for the treatment and/or prevention of toxicity induced by a T cell therapy wherein the T cell expresses at least one recombinant receptor. Preferably more than one IL-1 antagonist or a combination of IL-1 antagonists is used.

Preferably a) the administration of the IL-1 antagonist(s) is:
(i) at a time that is less than or no more than ten, seven, six, five, four or three days after initiation of the administration of the cell therapy; and/or
(ii) at a time at which the subject does not exhibit a sign or symptom of toxicity; and/or
(b) between the time of the initiation of the administration of the cell therapy and the time of the administration of the IL-1 antagonist, the subject has not exhibited toxicity; and/or
(c) the administration of the IL-1 antagonist is performed before or simultaneously to the T cell therapy.

Preferably the IL-1 antagonist(s) is selected from the group consisting of: anakinra, rilonacept, canakinumab, gevokizumab, LY2189102, MABp1, MEDI-8968, CYT013, sIL-1RI, sIL-1RII, EBI-005, CMPX-1023, VX-765 as reported and described in Table I below. Preferably the toxicity is selected from the group consisting of: cytokine release syndrome, neurotoxicity, delayed toxicity, preferably the neurotoxicity is severe neurotoxicity, preferably the severe neurotoxicity is a grade 3 or higher neurotoxicity.

Preferably the physical signs or symptoms associated with neurotoxicity, optionally severe neurotoxicity are selected from among confusion, delirium, expressive aphasia, obtundation, myoclonus, lethargy, altered mental status, convulsions, seizure-like activity, seizures (optionally as confirmed by electroencephalogram [EEG]), encephalopathy, dysphasia, tremor, choreoathetosis, symptoms that limit self-care, symptoms of peripheral motor neuropathy, symptoms of peripheral sensory neuropathy and combinations thereof; and/or the physical signs or symptoms associated with toxicity, optionally severe neurotoxicity, are associated with grade 3, grade 4 or grade 5 neurotoxicity; and/or the physical signs or symptoms associated with neurotoxicity, optionally severe neurotoxicity, manifest greater than or greater than about or about 5 days after cell therapy, 6 days after cell therapy or 7 days after T cell therapy.

Preferably the physical signs or symptoms associated with neurotoxicity, are selected from among acute inflammatory response and/or endothelial organ damage, fever, rigors, chills, hypotension, dyspnea, acute respiratory distress syndrome (ARDS), encephalopathy, ALT/AST elevation, renal failure, cardiac disorders, hypoxia, neurologic disturbances, and death, neurological complications such as delirium, seizure-like activity, confusion, word-finding difficulty, aphasia, and/or becoming obtunded, or fatigue, nausea, headache, seizure, tachycardia, myalgias, rash, acute vascular leak syndrome, liver function impairment, and renal failure and combinations thereof; and/or the physical signs or symptoms associated with toxicity manifest greater than or greater than about or about 5 days after cell therapy, 6 days after cell therapy or 7 days after cell therapy.

In a preferred embodiment the T cell therapy is for treating a disease or condition in the subject, which T cell therapy is associated with or is capable of inducing neurotoxicity, wherein the T cell therapy optionally is adoptive cell therapy and/or wherein the T cell therapy comprises administration of a dose of cells to treat a disease or condition in the subject.

Preferably the disease or condition is a cancer; preferably the disease or condition is a solid or an hematopoietic cancer, and/or the disease or condition is a leukemia or lymphoma; and/or the disease or condition is a non-Hodgkin lymphoma (NHL), preferably acute lymphoblastic leukemia (ALL).

Preferably the dose of T cells comprises a number of cells between about 0.5×10⁶ cells/kg body weight of the subject and 3×10⁶ cells/kg, between about 0.75×10⁶ cells/kg and 2.5×10⁶ cells/kg or between about 1×10⁶ cells/kg and 2×10⁶ cells/kg.

Still preferably the dose of T cells comprises a number of cells between about such as between about 1×10⁵ cells/kg and 5×10⁷ cells/kg, 2×10⁵ cells/kg and 2×10⁷cells/kg, 2×10⁵ cells/kg and 1×10⁷ cells/kg, 2×10⁵ cells/kg and 5×10⁶ cells/kg, 2×10⁵cells/kg and 2×10⁶ cells/kg or 2×10⁵ cells/kg and 1×10⁶ cells/kg.

The present invention also provides the IL-1 antagonist for use as indicated above in combination with a further therapeutic agent.

Preferably the further therapeutic agent is a IL-6 antagonist or a chemotherapeutic agent, preferably the further therapeutic agent is selected from among tocilizumab, siltuximab, sarilumab, clazakizumab, olokizumab (CDP6038), elsilimomab, ALD518/BMS-945429, sirukumab (CNTO 136), CPSI-2634, ARGX-109, FE301, FMIOI, Hu-Mik-β-I, tofacitinib, ruxolitinib, CCX140-B, R0523444, BMS CCR2 22, INCB 3284 dimesylate, JNJ27141491 and RS 504393, adalimumab, certolizumab pegol, golimumab, lenalidomide, ibrutinib or acalabrutinib.

Preferably the recombinant receptor as indicated above binds to, recognizes or targets an antigen associated with the disease or condition; and/or the recombinant receptor is a T cell receptor or a functional non-T cell receptor; and/or the recombinant receptor is a chimeric antigen receptor (CAR).

Preferably the CAR comprises an extracellular antigen-recognition domain that specifically binds to the antigen and an intracellular signaling domain comprising an IT AM, wherein optionally, the intracellular signaling domain comprises an intracellular domain of a CD3-zeta chain; and/or wherein the CAR further comprises a costimulatory signaling region, which optionally comprises a signaling domain of CD28 or 4-IBB.

Preferably the antigen is CD19 or CD 44v6. Preferably the T cell is a CD4+ or CD8+ T cell.

The present invention also provides a pharmaceutical composition comprising a IL-1 antagonist and pharmaceutically acceptable excipients for use for the treatment and/or prevention of toxicity induced by a T cell therapy wherein the T cell expresses at least one recombinant receptor. Preferably the pharmaceutical composition comprises at least one IL-1 antagonist or a combination thereof. Preferably the pharmaceutical composition further comprises a therapeutic agent. Preferably the further therapeutic agent is selected from the group consisting of: Il-6 antagonist or a chemotherapeutic agent, preferably the further therapeutic agent is selected from among tocilizumab, siltuximab, sarilumab, clazakizumab, olokizumab (CDP6038), elsilimomab, ALD518/BMS-945429, sirukumab (CNTO 136), CPSI-2634, ARGX-109, FE301, FMIOI, Hu-Mik-β-I, tofacitinib, ruxolitinib, CCX140-B, R0523444, BMS CCR2 22, INCB 3284 dimesylate, JNJ27141491 and RS 504393, adalimumab, certolizumab pegol, golimumab, lenalidomide, ibrutinib or acalabrutinib.

Preferably the pharmaceutical composition for use for the treatment and/or prevention of a toxicity selected from the group consisting of cytokine release syndrome, neurotoxicity, delayed toxicity, preferably the neurotoxicity is severe neurotoxicity, preferably the severe neurotoxicity is a grade 3 or higher neurotoxicity.

In some embodiments, the agent is an antagonist or inhibitor of IL-1 or of the IL-1 receptor (IL-1R). In some aspects, the agent is an IL-1 receptor antagonist, which is a modified form of IL-1R, such as anakinra (see, e.g., Fleischmann et al., (2006) Annals of the rheumatic diseases. 65(8): 1006-12). In some aspects, the agent is an antibody that neutralizes IL-1 activity, such as an antibody or antigen-binding fragment that binds to IL-1 or IL-1R, such as canakinumab (see also EP 2277543). In some embodiments, the agent that is an antagonist or inhibitor of IL-1/IL-1R is a small molecule, a protein or peptide, or a nucleic acid.

Preferably the at least one IL-1 antagonist is selected from any one as reported in Table I below:

TABLE I
Prefered IL-1 antagonists

| Agent | Availability | Mechanism of action | Company |
|---|---|---|---|
| Anakinra | Approved | Receptor antagonist for IL-1RI | Swedish Orphan BioVitrum (see |
| | | | Supplementary information S1 |
| | | | (table)) |
| Rilonacept ^{#} | Approved | Soluble IL-1 receptor that binds | Regeneron |
| | | IL-1β > IL-1α > IL-1Ra | |
| Canakinumab | Approved | Neutralizing anti-IL-1β IgG1 mAb | Novartis |
| Gevokizumab | Phase II | Neutralizing anti-IL-1β IgG2 mAb | Xoma |
| LY2189102 | Phase II | Neutralizing anti-IL-1β IgG1 mAb | Lilly |
| MABp1 | Phase I/II | Neutralizing anti-IL-1α IgG1 mAb | XBiotech |
| MEDI-8968 | Phase II/III | Blocking antibody to IL-1RI | MedImmune |
| CYT013 | Phase I | Therapeutic vaccine targeting IL-1β | Cytos Biotechnology |
| sIL-1RI^{‡} | Halted | Binds IL-1Ra > IL-1α > IL-1β | Amgen |
| sIL-1RII^{§} | Halted | Binds IL-1β complex with soluble IL-1RAcP | Amgen |
| EBI-005 | Phase I/II | Chimeric IL-1Ra-IL-1β | Eleven Biotherapeutics |
| CMPX-1023 | Preclinical | Alphabody | Complix |
| VX-765 | Phase II | Oral caspase 1 inhibitor Vertex | Vertex |

In the present invention the IL-1 antagonist treats, prevents, delays, or attenuates the development of a toxicity.

Provided in some aspects are methods of treatment including administering to a subject an IL-1 antagonist capable of treating, preventing, delaying, or attenuating the development of a toxicity. In some cases, at the time of said administration, the subject has been previously administered a cell therapy. In some embodiments, the administration of the IL-1 antagonist is at a time that is less than or no more than ten, seven, six, five, four or three days after initiation of the administration of the therapy. In some embodiments, the administration of the IL-1 antagonist is at a time at which the subject does not exhibit a sign or symptom of toxicity and/or does not exhibit grade 2 or higher toxicity (see Table II).

In some embodiments, the administration of the IL-1 antagonist is at a time at which the subject does not exhibit a sign or symptom of severe neurotoxicity and/or does not exhibit grade 2 or higher neurotoxicity. In some aspects, between the time of the initiation of the administration of the therapy and the time of the administration of the IL-1 antagonist the subject has not exhibited severe toxicity and/or has not exhibited grade 2 or higher toxicity. In some instances, between the time of the initiation of the administration of the cell therapy and the time of the administration of the IL-1 antagonist, the subject has not exhibited severe neurotoxicity and/or does not exhibit grade 2 or higher neurotoxicity.

Provided in some embodiments are methods of treatment including administering to a subject having a disease or condition a cell therapy. In some instances, the method includes administering to the subject an IL-1 antagonist capable of treating, preventing, delaying, or attenuating the development of a toxicity to the administered cell therapy at a time within 24 hours after the first sign of a toxicity following initiation of administration of the therapy. In some aspects, the IL-1 antagonist is administered within about 16 hours, within about 12 hours, within about 8 hours, within about 2 hours or within about 1 hour after the first sign of toxicity following initiation of administration of the therapy.

In some embodiments, the IL-1 antagonist is administered less than five days after initiation of administration of the therapy, less than four days after initiation of administration of the therapy or less than three days after initiation of administration of the therapy.

In some embodiments, the therapy is or comprises a cell therapy. In some cases, the cell therapy is or comprises an adoptive cell therapy. In some aspects, the therapy is or comprises a tumor infiltrating lymphocytic (TIL) therapy, a transgenic TCR therapy or a recombinant receptor-expressing cell therapy, which optionally is a T cell therapy. In some embodiments, the therapy is a chimeric antigen receptor (CAR)-expressing T cell therapy.

In some cases, the IL-1 antagonist is combined with an agent selected from among tocilizumab, situximab, sarilumab, olokizumab (CDP6038), elsilimomab, ALD518/BMS-945429, sirukumab (CNTO 136), CPSI-2634, ARGX-109, FE301 and FMIOI.

In some embodiments, tocilizumab is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive, or tocilizumab is administered in a dosage amount of at least or at least about or about 2 mg/kg, 4 mg/kg, 6 mg/kg or 8 mg/kg.

In some of any of the above embodiments, the therapy is or comprises a cell therapy and the number of cells administered is between about 0.25×10⁶ cells/kg body weight of the subject and 5×10⁶ cells/kg, 0.5×10⁶ cells/kg body weight of the subject and 3×10⁶ cells/kg, between about 0.75×10⁶ cells/kg and 2.5×10⁶ cells/kg or between about 1×10⁶ cells/kg and 2×10⁶ cells/kg, each inclusive.

In some embodiments, the therapy is or comprises a cell therapy and the cells are administered in a single pharmaceutical composition containing the cells. In some cases, the therapy is or comprises a cell therapy and the dose of cells is a split dose, wherein the cells of the dose are administered in a plurality of compositions, collectively containing the cells of the dose, over a period of no more than three days.

In some embodiments, the disease or condition is or comprises a tumor or a cancer. In some cases, the disease or condition is or comprises a leukemia or lymphoma. In some embodiments, the disease or condition is a B cell malignancy or is a hematological disease or condition. In some aspects, the disease or condition is or comprises a non-Hodgkin lymphoma (NHL) or acute lymphoblastic leukemia (ALL).

In some embodiments, the therapy is a cell therapy including a dose of cells expressing a recombinant receptor. In some aspects, the recombinant receptor binds to, recognizes or targets an antigen associated with the disease or condition. In some cases, the recombinant receptor is a T cell receptor or a functional non-T cell receptor. In some instances, the recombinant receptor is a chimeric antigen receptor (CAR).

In some embodiments, the CAR contains an extracellular antigen-recognition domain that specifically binds to the antigen and an intracellular signaling domain containing an IT AM. In some cases, the antigen is CD 19 or CD44v6. In some embodiments, the intracellular signaling domain contains an intracellular domain of a CD3-zeta chain. In some embodiments, the CAR further contains a costimulatory signaling region. In some aspects, the costimulatory signaling domain contains a signaling domain of CD28 or 4-1BB.

In some embodiments, the therapy is or comprises a therapy containing a dose of cells containing T cells. In some cases, the T cells are CD4+ or CD8+. In some embodiments, the T cells are autologous to the subject. In some embodiments, the method further includes administering a chemotherapeutic agent prior to administering the therapy. In some instances, the subject has been previously treated with a chemotherapeutic agent prior to the initiation of administration of the therapy. In some aspects, the chemotherapeutic agent includes an agent selected from the group consisting of cyclophosphamide, fludarabine, and/or a combination thereof. In some embodiments, the chemotherapeutic agent is administered between 2 and 5 days prior to the initiation of administration of the therapy. In some cases, the chemotherapeutic agent is administered at a dose of between at or about 1 g/m² of the subject and at or about 3 g/m² of the subject.

In some embodiments, toxicity is a neurotoxicity. In some embodiments, a CNS-related outcome in the subject at day up to or up to about day 7, 8, 9, 10, 11, 12, 13, 14, 15, 16, 17, 18, 19, 20, 21, 22, 23, 24, 25, 26, 27, 28, 29 or 30 following administration of the therapy is not detectable or is reduced as compared to a method including an alternative treatment regimen wherein the subject is administered the IL-1 antagonist after severe neurotoxicity has developed or after grade 2 or higher neurotoxicity has developed. In some embodiments, the toxic outcome is a symptom associated with grade 3 or higher neurotoxicity. In some embodiments, the toxic outcome is reduced by greater than 50%, 60%, 70%, 80%, 90% or more. In some cases, the toxic outcome is a symptom associated with grade 3 or higher neurotoxicity. In some embodiments, the toxic outcome is selected from among grade 3 or higher neurotoxicity include confusion, delirium, expressive aphasia, obtundation, myoclonus, lethargy, altered mental status, convulsions, seizure-like activity and seizures. In some aspects, in the cell therapy, the cells exhibit increased or longer expansion and/or persistence in the subject than cells administered in a method including an alternative treatment regimen wherein the subject is administered the agent or other treatment after severe neurotoxicity has developed or after grade 2 or higher neurotoxicity has developed. In some instances, expansion and/or persistence is increased 2-fold, 3-fold, 4-fold, 5-fold, 6-fold, 7-fold, 8-fold, 9-fold or 10-fold.

In some embodiments, the cell therapy, comprises engineered and/or CAR-expressing cells. In some cases, the concentration or number of the engineered and/or CAR-expressing cells in the blood of the subject at day 30, day 60, or day 90 following initiation of administration of the therapy is at least at or about 10 engineered or CAR-expressing cells per microliter, at least 50% of the total number of peripheral blood mononuclear cells (PBMCs), at least or at least about 1×10⁵ engineered or CAR-expressing cells, and/or at least 5,000 copies of CAR-encoding or engineered receptor-encoding DNA per micrograms DNA. In some embodiments, at day 30, 60, or 90 following the initiation of the administration of the therapy, the CAR-expressing and/or engineered cells are detectable in the blood or serum of the subject. In some instances, at day 30, 60, or 90 following the initiation of the administration of the therapy, the blood of the subject contains at least 20% CAR-expressing cells, at least 10 CAR-expressing cells per microliter or at least 1×10⁴ CAR-expressing cells. In some cases, at day 30, 60, or 90 following the initiation of the administration of the therapy, the blood of the subject contains at least 50%, 60%, 70%, 80%, or 90% of a biologically effective dose of the cells. In some embodiments, at day 30, 60, or 90 following the initiation of the administration of the therapy, the blood of the subject contains at least 20% engineered and/or CAR-expressing cells, at least 10 engineered and/or CAR-expressing cells per microliter and/or at least 1×10⁴ engineered and/or CAR-expressing cells. In some cases, at day 30, 60, or 90 following the initiation of the administration of the therapy, the subject exhibits a reduction or sustained reduction in burden of the disease or condition. In some cases, the reduction or sustained reduction in burden of the disease or condition is at or about or at least at or about 50, 60, 70, or 80% peak reduction following the therapy administration or reduction associated with effective dose.

In some embodiments, at day 30, 60 or 90 following the initiation of the administration of the therapy, the subject does not, and/or has not, following the cell therapy treatment, exhibited severe neurotoxicity, grade 2 or higher neurotoxicity, and/or has not exhibited seizures or other CNS outcome; or at day 30, 60, or 90 following the initiation of the administration of the therapy, less than or about less than 25%, less than or about less than 20%, less than or about less than 15%, or less than or about less than 10%) of the subjects so treated do not, and/or have not, following the cell therapy treatment, exhibited severe neurotoxicity, grade 2 or higher neurotoxicity, and/or have not exhibited seizures or other CNS outcome. In some embodiments, the cell therapy, comprising engineered and/or CAR-expressing cells; and the area under the curve (AUC) for blood concentration of engineered and/or CAR-expressing cells over time following the administration of the therapy is greater as compared to that achieved via a method comprising an alternative dosing regimen, such as where the subject is administered the therapy and is administered the IL-1 antagonist at a time at which the subject exhibits a severe or grade 2 or higher or grade 3 or higher neurotoxicity.

In some embodiments, symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03).

In some embodiments, neurologic symptoms are seen to begin 5 to 7 days after cell therapy infusion. In some embodiments, duration of neurologic changes may range from 3 to 19 days. In some cases, recovery of neurologic changes occurs after other symptoms of sCRS have resolved. In some embodiments, time or degree of resolution of neurologic changes is not hastened by treatment with anti-IL-6 and/or steroid(s).

In some embodiments, a subject is deemed to develop “severe neurotoxicity” in response to or secondary to administration of a cell therapy or dose of cells thereof, if, following administration, the subject displays symptoms that limit self-care (e.g. bathing, dressing and undressing, feeding, using the toilet, taking medications) from among: 1) symptoms of peripheral motor neuropathy, including inflammation or degeneration of the peripheral motor nerves; 2) symptoms of peripheral sensory neuropathy, including inflammation or degeneration of the peripheral sensory nerves, dysesthesia, such as distortion of sensory perception, resulting in an abnormal and unpleasant sensation, neuralgia, such as intense painful sensation along a nerve or a group of nerves, and/or paresthesia, such as functional disturbances of sensory neurons resulting in abnormal cutaneous sensations of tingling, numbness, pressure, cold and warmth in the absence of stimulus. In some embodiments, severe neurotoxicity includes neurotoxicity with a grade of 3 or greater, such as set forth in Table II.

TABLE II
Exemplary Grading Criteria for neurotoxicity

| Grade | Description of Symptoms |
|---|---|
| 1 | Mild or asymptomatic symptoms |
| Asymptomatic | |
| or Mild | |
| 2 | Presence of symptoms that limit instrumental activities |
| Moderate | of daily living (ADL), such as preparing meals, |
| | shopping for groceries or clothes, using the telephone, |
| | managing money |
| 3 | Presence of symptoms that limit self-care ADL, such |
| Severe | as bathing, dressing and undressing, feeding self, using |
| | the toilet, taking medications |
| 4 | Symptoms that are life-threatening, requiring urgent |
| Life-threatening | intervention |
| 5 | Death |
| Fatal | |

In some embodiments, the methods reduce symptoms associated with CNS-outcomes or neurotoxicity compared to other methods. For example, subjects treated according to the present methods may lack detectable and/or hpve reduced symptoms of neurotoxicity, such as limb weakness or numbness, loss of memory, vision, and/or intellect, uncontrollable obsessive and/or compulsive behaviors, delusions, headache, cognitive and behavioral problems including loss of motor control, cognitive deterioration, and autonomic nervous system dysfunction, and sexual dysfunction, compared to subjects treated by other methods in which the administration of the toxicity-targeting agent is administered later and after severe CRS or severe neurotoxicity or other toxic outcomes have developed. In some embodiments, subjects treated according to the present methods may have reduced symptoms associated with peripheral motor neuropathy, peripheral sensory neuropathy, dysethesia, neuralgia or paresthesia. In some embodiments, the methods reduce outcomes associated with neurotoxicity including damages to the nervous system and/or brain, such as the death of neurons. In some aspects, the methods reduce the level of factors associated with neurotoxicity such as beta amyloid (Aβ), glutamate, and oxygen radicals.

In some embodiments, subjects administered the therapy in conjunction with an early intervention with a IL-1 antagonist have reduced symptoms, outcomes, or factors associated with a CNS-related outcome or neurotoxicity (e.g. severe neurotoxicity or grade 3 or higher neurotoxcity) compared to a method comprising an alternative treatment regimen wherein the subject is administered the IL-1 antagonist after grade 2 or higher neurotoxicity has developed. In some embodiments, the CNS-related or neurotoxicity (e.g. severe neurotoxicity or grade 3 or higher neurotoxicity) outcome is reduced by greater than 50%, 60%, 70%, 80%, 90% or more. In some embodiments, administration of the cell therapy causes one more adverse events. In some embodiments, the adverse event includes, but is not limited to, an increase in alanine aminotransferase, an increase in aspartate aminotransferase, chills, febrile neutropenia, headache, hypotension, left ventricular dysfunction, encephalopathy, hydrocephalus, seizure, and/or tremor. In some embodiments, the intervention methods provided herein ameliorate or reduce such adverse events.

Cell Therapy and Engineered Cells

In some aspects, the provided therapeutic methods involve administering cells expressing a recombinant receptor, and compositions thereof, to subjects, e.g., patients. In some embodiments, the cells contain or are engineered to contain an engineered receptor, e.g., an engineered antigen receptor, such as a chimeric antigen receptor (CAR), or a T cell receptor (TCR). The cells include populations of such cells, compositions containing such cells and/or enriched for such cells, such as in which cells of a certain type such as T cells or CD8+ or CD4+ cells are enriched or selected. Among the compositions are pharmaceutical compositions and formulations for administration, such as for adoptive cell therapy. In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

Recombinant Receptors

The cells generally express recombinant receptors, such as antigen receptors including functional non-TCR antigen receptors, e.g., chimeric antigen receptors (CARs), and other antigen-binding receptors such as transgenic T cell receptors (TCRs). Also, among the receptors are other chimeric receptors.

Chimeric Antigen Receptors (CARs)

Exemplary antigen receptors, including CARs, and methods for engineering and introducing such receptors into cells, include those described, for example, in International Patent Application Publication Numbers WO200014257, W2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479, 118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include orphan tyrosine kinase receptor ROR1, tEGFR, Her2, LI-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, KG2D Ligands, NY-ESO-1, MART-1, gpIOO, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD 123, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgGI. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in International Patent Application Publication Number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, International Patent Application Publication Number WO2014031687, U.S. Pat. No. 8,822,647 or published app. No. US2014/0271635.

In some embodiments, the constant region or portion is of a human IgG, such as IgG4 or IgGI. This antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD 16. For example, in some aspects, the CAR or other chimeric receptor includes a chimeric molecule between CD3-zeta or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement. In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of IT AM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-IBB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects. In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-IBB, T FRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-IBB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in International Patent Application Publication Number WO2014031687. For example, the marker can be a truncated EGFR (tEGFR) that is, optionally, linked to a linker sequence, such as a T2A cleavable linker sequence. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors. In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P01747.1) or variant thereof.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein.

In some embodiments, the intracellular signaling domain of the recombinant receptor, e.g. the CAR, comprises a human CD3 zeta stimulatory signaling domain or functional variant thereof, such as an 112 AA cytoplasmic domain of isoform 3 of human CD3-zeta (Accession No.: P20963.2) or a CD3 zeta signaling domain as described in U.S. Pat. Nos. 7,446,190 or 8,911,993.

In some aspects, the spacer contains only a hinge region of an IgG, such as only a hinge of IgG4 or IgGI. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-IBB-derived intracellular signaling domain, and a CD3 zeta-derived signaling domain. In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence. The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

TCRs

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13: 1050-1063; Frecha et al. (2010) Molecular Therapy: The Journal of the American Society of Gene Therapy. 18: 1748-1757; Hackett et al. (2010) Molecular Therapy: The Journal of the American Society of Gene Therapy. 18:674-683.

Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory

CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing IT AM or ITAM-like motifs. In some embodiments, the activation induced by the first receptor involves a signal transduction or change in protein expression in the cell resulting in initiation of an immune response, such as IT AM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-KB and/or AP-1, and/or induction of gene expression of factors such as cytokines, proliferation, and/or survival. In some embodiments, the first and/or second receptor includes intracellular signaling domains of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-IBB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved. In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

Cells and Preparation of Cells for Genetic Engineering

Among the cells expressing the receptors and administered in the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or K cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and reintroducing them into the same subject, before or after cryopreservation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the cells are natural killer (K) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types. In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 antibody conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy. In embodiments, memory T cells are present in both CD62L+ and CD62L⁻ subsets of CD8+ peripheral blood lymphocytes. PBMC can be enriched for or depleted of CD62L−CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subj ected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order. CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA, CD62L, CD4+T cells. In some embodiments, central memory CD4 cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample. In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells. In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application Publication Number WO2009/072003, or US Patent Application Publication Number US 20110003380.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps. In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701. In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., International Patent Application Publication Number WO 2010/033140, Cho et al. (2010) Lab Chip 10, 1567-1573; and Godin et al. (2008) J Biophoton. I(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously. In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL. In some aspects, the IL-2 concentration is at least about 10 units/mL. In some embodiments, the stimulating agents include PMA and ionomycin. In some aspects, incubation is carried out in accordance with techniques such as those described in U.S. Pat. No. 6,040, 177 to Riddell et al., Klebanoff et al. (2012) J Immunother. 35(9): 651-660, Terakura et al. (2012) Blood. 1:72-82, and/or Wang et al. (2012) J Immunother. 35 (9): 689-701.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Vectors and Methods for Genetic Engineering

Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in International Patent Application Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing. In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol and Cell Biol, 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17. In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example, in some aspects, the cells are engineered so that they can be eliminated as a result of a change in the in vivo condition of the subject to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

Compositions and Formulations

In some embodiments, the immunotherapy and/or a cell therapy is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods. The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some embodiments, the T cell therapy, such as engineered T cells (e.g. CAR T cells), are formulated with a pharmaceutically acceptable carrier. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride;

hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine;monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells, including one or more active ingredients where the activities are complementary to the cells and/or the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contain cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

Prolonged absorption of the injectable pharmaceutical form can be brought about by the use of agents delaying absorption, for example, aluminum monostearate and gelatin. The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

Treatment and Methods

In some embodiments, the immunotherapy and/or a cell therapy, e.g., a dose of cells expressing a recombinant receptor are administered to a subject to treat or prevent diseases, conditions, and disorders, including cancers. In some embodiments, the immunotherapy and/or a cell therapy, e.g., cells, populations, and compositions are administered to a subject or patient having the particular disease or condition to be treated, e.g., via adoptive cell therapy, such as adoptive T cell therapy. In some embodiments, cells and compositions, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell. In some embodiments, the provided methods include an early or preemptive intervention or interventions, including by administration of agents or therapies or other treatments that are administered in addition to the immunotherapy and/or cell therapy.

Methods for administration of cells for adoptive cell therapy are known and may be used in connection with the provided methods and compositions. For example, adoptive T cell therapy methods are described, e.g., in US Patent Application Publication No. 2003/0170238 to Gmenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma. In some embodiments, the subject has acute-lymphoblastic leukemia (ALL). In some embodiments, the subject has non-Hodgkin's lymphoma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant. In some embodiments, the antigen associated with the disease or disorder is selected from the group consisting of orphan tyrosine kinase receptor ROR1, tEGFR, Her2, LI-CAM, CD 19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethy choline e receptor, GD2, GD3, HMW-MAA, IL-22R-alpha, IL-13R-alpha2, kdr, kappa light chain, Lewis Y, LI-cell adhesion molecule, MAGE-A1, mesothelin, MUC1, MUC16, PSCA, KG2D Ligands, NY-ESO-1, MART-1, gpIOO, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin AI (CCNA1), and/or biotinylated molecules, and/or molecules expressed by HIV, HCV, HBV or other pathogens.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the methods comprise administration of a chemotherapeutic agent, e.g., a conditioning chemotherapeutic agent, for example, to reduce tumor burden prior to the administration. Preconditioning subjects with immunodepleting (e.g., lymphodepleting) therapies in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy. In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose. Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, TNFγ, IL-2, and TNF. In some aspects the biological activity is measured by assessing clinical outcome, such as reduction in tumor burden or load. In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3:111 (1995), and U.S. Pat. No. 5,087,616.

Dosing

The pharmaceutical composition in some embodiments of the methods provided herein contains the cells in amounts effective to treat or prevent the disease or condition, such as a therapeutically effective or prophylactically effective amount. In some embodiments, the composition includes the cells in an amount effective to reduce burden of the disease or condition. In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the first dose.

The term “split dose” refers to a dose that is split so that it is administered over more than one day. This type of dosing is encompassed by the present methods and is considered to be a single dose.

Thus, the dose in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33%> of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, one or more consecutive or subsequent dose of cells can be administered to the subject. In some embodiments, the consecutive or subsequent dose of cells is administered greater than or greater than about 7 days, 14 days, 21 days, 28 days or 35 days after initiation of administration of the first dose of cells. The consecutive or subsequent dose of cells can be more than, approximately the same as, or less than the first dose. In some embodiments, administration of the T cell therapy, such as administration of the first and/or second dose of cells, can be repeated.

In some embodiments, a dose of cells is administered to subjects in accord with the provided methods. In some embodiments, the size or timing of the doses is determined as a function of the particular disease or condition in the subject. It is within the level of a skilled artisan to empirically determine the size or timing of the doses for a particular disease. Dosages may vary depending on attributes particular to the disease or disorder and/or patient and/or other treatments. In certain embodiments, the cells, or individual populations of sub-types of cells, are administered to the subject at a range of about 0.1 million to about 100 billion cells and/or that amount of cells per kilogram of body weight of the subject, such as, e.g., about 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subj ect. Dosages may vary depending on attributes particular to the disease or disorder and/or patient and/or other treatments. In some embodiments, such values refer to numbers of recombinant receptor expressing cells; in other embodiments, they refer to number of T cells or PBMCs or total cells administered. In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵cells/kg, no more than at or about 4×10⁵cells/kg, no more than at or about 5×10⁵cells/kg, no more than at or about 6×10⁵cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1,1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%), about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

The amount of the IL-1 antagonist that treats or ameliorates symptoms of a toxicity of a cell therapy, such neurotoxicity to be administered to ameliorate symptoms or adverse effects of a toxicity to a cell therapy, such as neurotoxicity, can be determined by standard clinical techniques. Exemplary adverse events include, but are not limited to, an increase in alanine aminotransferase, an increase in aspartate aminotransferase, chills, febrile neutropenia, headache, hypotension, left ventricular dysfunction, encephalopathy, hydrocephalus, seizure, and/or tremor.

In some embodiments, the IL-1 antagonist is administered in a dosage amount of from or from about 30 mg to 5000 mg, such as 50 mg to 1000 mg, 50 mg to 500 mg, 50 mg to 200 mg, 50 mg to 100 mg, 100 mg to 1000 mg, 100 mg to 500 mg, 100 mg to 200 mg, 200 mg to 1000 mg, 200 mg to 500 mg or 500 mg to 1000 mg. In some embodiments, the IL-1 antagonist is administered from or from about 0.5 mg/kg to 100 mg/kg, such as from or from about 1 mg/kg to 50 mg/kg, 1 mg/kg to 25 mg/kg, 1 mg/kg to 10 mg/kg, 1 mg/kg to 5 mg/kg, 5 mg/kg to 100 mg/kg, 5 mg/kg to 50 mg/kg, 5 mg/kg to 25 mg/kg, 5 mg/kg to 10 mg/kg, 10 mg/kg to 100 mg/kg, 10 mg/kg to 50 mg/kg, 10 mg/kg to 25 mg/kg, 25 mg/kg to 100 mg/kg, 25 mg/kg to 50 mg/kg to 50 mg/kg to 100 mg/kg. In some embodiments, the agent is administered in a dosage amount of from or from about 1 mg/kg to 10 mg/kg, 2 mg/kg to 8 mg/kg, 2 mg/kg to 6 mg/kg, 2 mg/kg to 4 mg/kg or 6 mg/kg to 8 mg/kg, each inclusive. In some aspects, the agent is administered in a dosage amount of at least or at least about or about 1 mg/kg, 2 mg/kg, 4 mg/kg, 6 mg/kg, 8 mg/kg, 10 mg/kg or more. In some embodiments, the agent is administered at a dose of 4 mg/kg or 8 mg/kg.

In some embodiments, the IL-1 antagonist is administered by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, the amount of the IL-1 antagonist is administered about or approximately twice daily, daily, every other day, three times a week, weekly, every other week or once a month.

In some embodiments, the IL-1 antagonist is administered as part of a composition or formulation, such as a pharmaceutical composition or formulation as described below. Thus, in some cases, the composition comprising the agent is administered as described below. In other aspects, the IL-1 antagonist is administered alone and may be administered by any known acceptable route of administration or by one described herein, such as with respect to compositions and pharmaceutical formulations.

In some embodiments, the IL-1 antagonist that treats or ameliorates symptoms of a toxicity of the cell therapy, such as neurotoxicity, is an antibody or antigen binding fragment. In some embodiments, the IL-1 antagonist is combined with tocilizumab, siltuximab, sarilumab, olokizumab (CDP6038), elsilimomab, ALD518/BMS-945429, sirukumab (CNTO 136), CPSI-2634, ARGX-109, FE301, or FMIOI.

In some embodiments, the IL-1 antagonist is combined with an antagonist or inhibitor of IL-6 or the IL-6 receptor (IL-6R), preferably an antibody that neutralizes IL-6 activity, such as an antibody or antigen-binding fragment that binds to IL-6 or IL-6R. For example, in some embodiments, the IL-1 antagonist is combined with tocilizumab (atlizumab) or sarilumab, anti-IL-6R antibodies. In some embodiments, the IL-1 antagonist is combined with an anti-IL-6R antibody described in U.S. Pat. No. 8,562,991, preferably siltuximab, elsilimomab, ALD518/BMS-945429, sirukumab (CNTO 136), CPSI-2634, ARGX-109, FE301, FMIOI, or olokizumab (CDP6038). In particular tocilizumab is administered as an early invervention in accord with the provided methods a dosage of from or from about 1 mg/kg to 12 mg/kg, such as at or about 4 mg/kg, 8 mg/kg, or 10 mg/kg. In some embodiments, tocilizumab is administered by intravenous infusion. In some embodiments, tocilizumab is administered for a persistent fever of greater than 39° C. lasting 10 hours that is unresponsive to acetaminophen. In some embodiments, a second administration of tocilizumab is provided if symptoms recur after 48 hours of the initial dose.

In some embodiments, the IL-1 antagonist is combined with an agonist or stimulator of TGF-β or a TGF-β receptor (e.g., TGF-β receptor I, II, or III), preferably an antibody that increases TGF-β activity, such as an antibody or antigen-binding fragment that binds to TGF-β or one of its receptors. In some embodiments, the agent that is an agonist or stimulator of TGF-β and/or its receptor is a small molecule, a protein or peptide, or a nucleic acid. In some embodiments, the agent is an antagonist or inhibitor of MCP-1 (CCL2) or a MCP-1 receptor (e.g., MCP-1 receptor CCR2 or CCR4). In some aspects, the agent is an antibody that neutralizes MCP-1 activity, such as an antibody or antigen-binding fragment that binds to MCP-1 or one of its receptors (CCR2 or CCR4). In some embodiments, the MCP-1 antagonist or inhibitor is any described in Gong et al. J Exp Med. 1997 Jul. 7; 186(1): 131-137 or Shahrara et al. J Immunol 2008; 180:3447-3456. In some embodiments, the agent that is an antagonist or inhibitor of MCP-1 and/or its receptor (CCR2 or CCR4) is a small molecule, a protein or peptide, or a nucleic acid.

In some embodiments, the agent is an antagonist or inhibitor of IFN-γ or an IFN-γ receptor (IFNGR). In some aspects, the agent is an antibody that neutralizes IFN-γ activity, such as an antibody or antigen-binding fragment that binds to IFN-γ or its receptor (IFNGR). In some aspects, the IFN-gamma neutralizing antibody is any described in Dobber et al. Cell Immunol. 1995 February; 160(2): 185-92 or Ozmen et al. J Immunol. 1993 Apr. 1; 150(7):2698-705. In some embodiments, the agent that is an antagonist or inhibitor of IFN-γ/IFNGR is a small molecule, a protein or peptide, or a nucleic acid.

In some embodiments, the agent is an antagonist or inhibitor of IL-10 or the IL-10 receptor (IL-IOR). In some aspects, the agent is an antibody that neutralizes IL-10 activity, such as an antibody or antigen-binding fragment that binds to IL-10 or IL-10R. In some aspects, the IL-10 neutralizing antibody is any described in Dobber et al. Cell Immunol. 1995 Febraury; 160(2): 185-92 or Hunter et al. J Immunol. 2005 Jun. 1; 174(11):7368-75. In some embodiments, the agent that is an antagonist or inhibitor of IL-101IL-IOR is a small molecule, a protein or peptide, or a nucleic acid.

Compositions and Formulations

In some embodiments, the agents, e.g., toxicity-targeting agents are provided as a composition or formulation, such as a pharmaceutical composition or formulation. Such compositions can be used in accord with the provided methods, such as in an early intervention for the prevention, treatment or amelioration of a toxicity, such as to delay, attenuate, reduce neurotoxicity in the subject.

In some embodiments, the toxicity-targeting agents are formulated with a pharmaceutical carrier. Such carriers can include, for example, carriers such as a diluent, adjuvant, excipient, or vehicle with which the agent is administered. Examples of suitable pharmaceutical carriers are described in “Remington's Pharmaceutical Sciences” by E. W. Martin. Such compositions will contain a therapeutically effective amount of the agent, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Saline solutions and aqueous dextrose and glycerol solutions also can be employed as liquid carriers, particularly for injectable solutions. The pharmaceutical compositions can contain any one or more of a diluents(s), adjuvant(s), antiadherent(s), binder(s), coating(s), filler(s), flavor(s), color(s), lubricant(s), glidant(s), preservative(s), detergent(s), sorbent(s), emulsifying agent(s), pharmaceutical excipient(s), pH buffering agent(s), or sweetener(s) and a combination thereof. In some embodiments, the pharmaceutical composition can be liquid, solid, a lyophilized powder, in gel form, and/or combination thereof. In some aspects, the choice of carrier is determined in part by the particular agent and/or by the method of administration. In some embodiments, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the agent to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369. The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician. The pharmaceutical composition in some embodiments contains agents in amounts effective to ameliorate the toxicity and/or to prevent, delay, or attenuate the development of or risk for developing a toxicity, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of toxicity or symptoms associated with toxicity occurs and/or the risk for developing the toxicity has passed. However, other dosage regimens may be useful and can be determined. The desired dosage can be delivered by a single bolus administration of the composition, by multiple bolus administrations of the composition, or by continuous infusion administration of the composition. The agents can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the agent. In some embodiments, it is administered by multiple bolus administrations of the agent. For the amelioration of a toxicity and/or to delay, attenuate to prevent the risk of a toxicity, the appropriate dosage may depend on the type of toxicity to be treated, the type of agent or agents, the type of cells or recombinant receptors previously administered to the subject, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. When administering a therapeutic composition (e.g., a pharmaceutical composition containing an agent that treats or ameliorates symptoms of a toxicity, such as CRS or neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent is administered parenterally. In some embodiments, the agent is administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the agent in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

Prolonged absorption of the injectable pharmaceutical form can be brought about by the use of agents delaying absorption, for example, aluminum monostearate and gelatin. Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes. In some embodiments, the toxicity-targeting agents are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. In some embodiments, unit dosage forms, include, but are not limited to, tablets, capsules, pills, powders, granules, sterile parenteral solutions or suspensions, and oral solutions or suspensions, and oil water emulsions containing suitable quantities of the compounds or pharmaceutically acceptable derivatives thereof. Unit dose forms can be contained ampoules and syringes or individually packaged tablets or capsules. Unit dose forms can be administered in fractions or multiples thereof. In some embodiments, a multiple dose form is a plurality of identical unit dosage forms packaged in a single container to be administered in segregated unit dose form. Examples of multiple dose forms include vials, bottles of tablets or capsules or bottles of pints or gallons.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

All publications mentioned in the above specification are herein incorporated by reference. Various modifications and variations of the described CARS (in particular WO 2016/042461), polynucleotides, vectors, cells and compositions of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

The present invention will be illustrated by means of non-limiting examples in reference to the following figures.

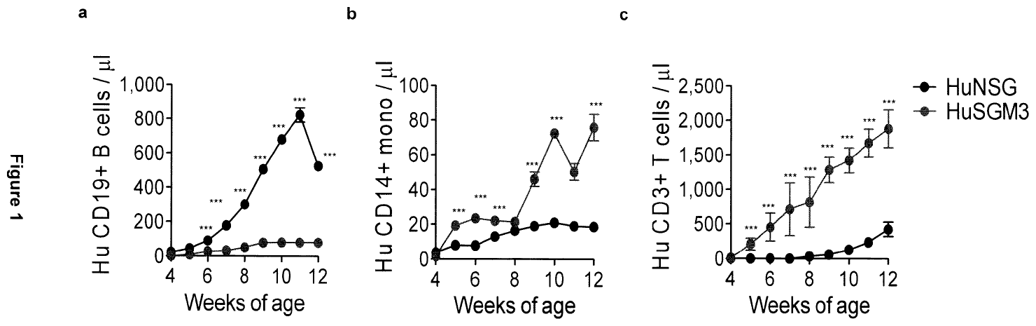

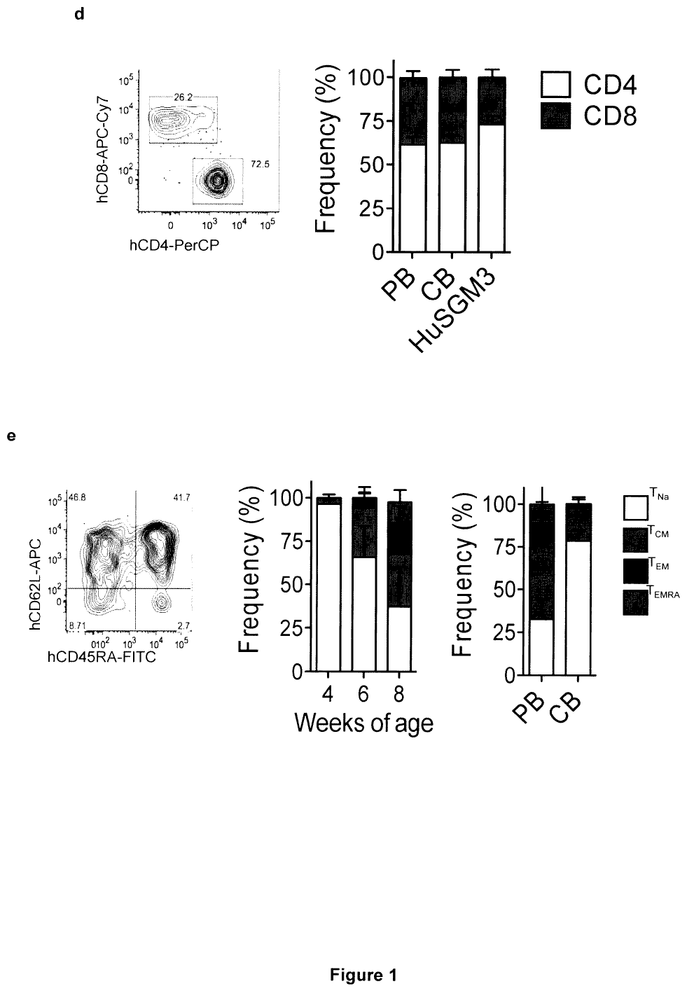

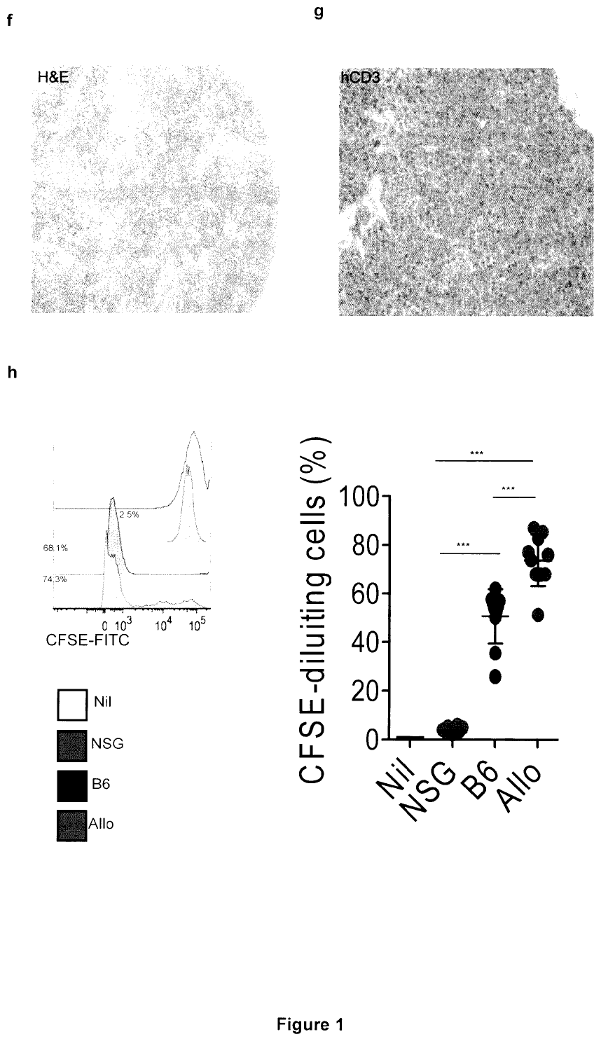

FIG. 1. Non-xenoreactive HuSGM3 T cells can be redirected against leukemia by CAR gene transfer. Cord blood (CB) human CD34⁺ hematopoietic stem cells (HSCs, n=5 donors) were injected intra-liver into irradiated newborn NSG (n=10, HuNSG) or SGM3 (n=10, HuSGM3) mice. After weaning, mice were monitored weekly for human lympho-hematopoietic reconstitution. (a) Mean counts±SD of human (Hu) CD19⁺ B cells, (b) CD14⁺ monocytes or (c) CD3⁺ T cells in mice over time (weeks of age) are shown. (d) Representative plot of circulating human CD4/CD8 T cells in HuSGM3 mice at 8 weeks of age (left) and mean CD4/CD8 frequencies±SD in human peripheral blood (PB, n=16 donors), CB (n=12 donors) and HuSGM3 T cells (right) are shown. (e) Representative plot of circulating human CD45RA/CD62L T cells in HuSGM3 at 8 weeks of age (left), mean frequencies±SD of circulating CD45RA⁺/CD62L⁺ naïve/stem cell memory (T_{Na/SCM}), CD45RA⁻/CD62L⁺ central memory (T_{CM}), CD45RA⁻/CD62L⁻ effector memory (T_{EM}) and CD45RA⁺/CD62L⁻ effector memory RA (T_{EMRA}) cells in HuSGM3 mice at 4, 6 and 8 weeks of age (middle) and in PB and CB (right) are shown. (f) Histology (hematoxylin and eosin, H&E) and (g) human CD3 immunohistochemistry pictures of HuSGM3 mouse thymus at 12 weeks of age (representative of n=5) are shown. (h) T cells were harvested from the spleen of 12-weeks old HuSGM3 mice (n=9) and left alone (Nil) or co-cultured with irradiated splenocytes from NSG or C57/Bl6 (B6) mice, or with irradiated human allogeneic PB mononuclear cells (Allo). Proliferation of HuSGM3 T cells was measured by CFSE-dilution. Representative plots (left) and percentages of CFSE-diluting cells in response to the different stimuli (right) are shown. Dots represent biological replicates. (i-I) 5×10⁶ HuSGM3 or PB T cells were infused into sub-lethally irradiated NSG mice (n=15 per group from three independent experiments). Mean percentages±SD of weight from initial and of circulating human CD3+ T cells over weeks from T cells are shown. (m) 5×10⁶ HuSGM3 T cells were transferred into sub-lethally irradiated NSG mice (n=18 from two independent experiments). After 24 weeks, mice were challenged with irradiated DCs from NSG mice (NSG, n=6), human allogeneic PB (Allo, n=6) or autologous CB mononuclear cells (Auto, n=6). Mice were re-challenged after 48 days (arrow). Mean percentages±SD of circulating human CD3+ T cells over days from DCs are shown. (n) HuSGM3 T cells were activated with CD3/CD28-beads and IL-7/IL-15, and RV transduced with either a CD44v6.28z, a CD44v6.BBz or a CD44v6.zOX CAR (see Methods). HuSGM3 CAR-T cells were co-cultured at a 1:10 E:T ratio with CD33⁺CD44v6⁺ THP-1 leukemic cells (upper row) or with CD19⁺CD44v6⁻ BV173 leukemic cells (lower row). Representative plots after 4-days co-culture (left) and mean elimination indexes±SD (see Methods) by CD44v6 CAR-T cells of different design from 9 independent experiments (right panel) are shown. Results from a one- or a two-way ANOVA test are indicated when statistically significant (*, P<0.05; **, P<0.01; ***, P<0.001).

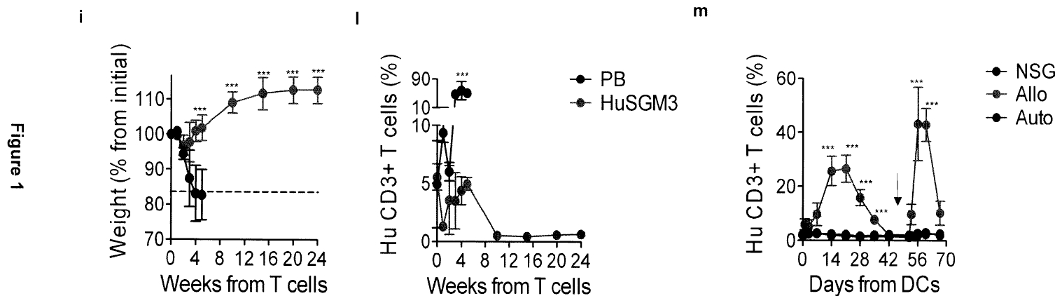

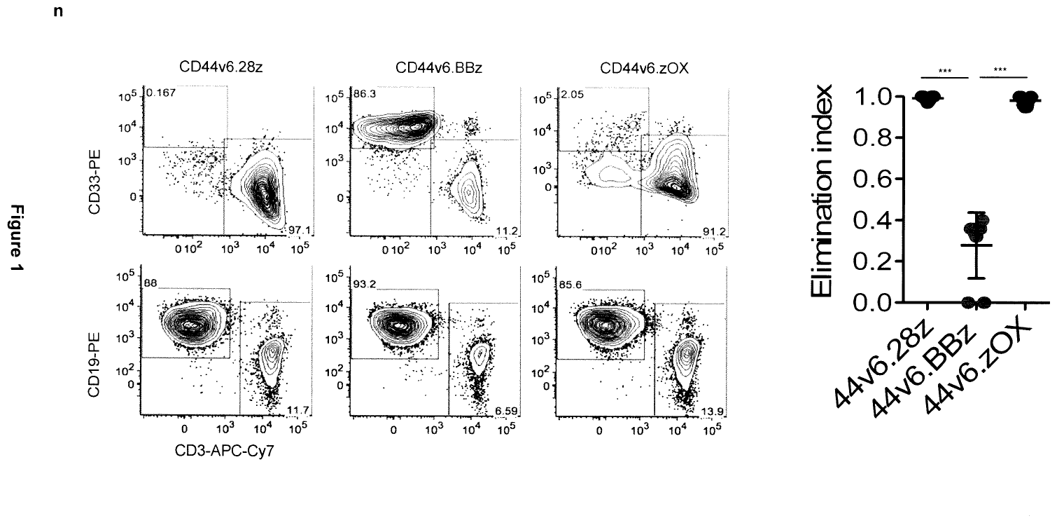

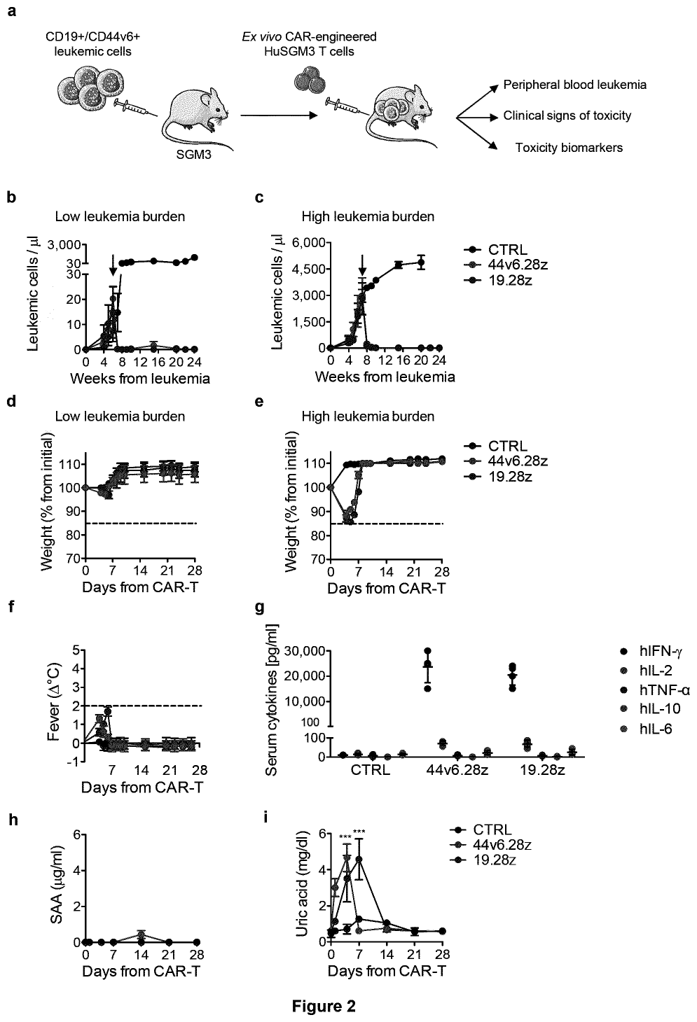

FIG. 2. Non-xenoreactive CAR-T cells cause TLS in SGM3 mice. (a) Adult SGM3 mice (8 weeks of age) were infused i.v. with 5×10⁶ (low leukemia burden) or 10×10⁶ (high leukemia burden) CD19⁺CD44v6⁺ ALL-CM leukemic cells and after 5 weeks (low leukemia burden) or 7 weeks (high leukemia burden) with 2×10⁶ T cells from newborn HuSGM3 mice (n=3 HSC donors) that had been ex vivo engineered with either a CD44v6.28z CAR (44v6.28z, n=12 mice from two independent experiments), a CD19.28z CAR (19.28z, n=12 mice) or left untransduced (CTRL, n=10 mice). Secondary recipients were followed over time by daily monitoring of weight loss and body temperature, and weekly monitoring of serum concentrations of human cytokines, mouse amyloid A (SAA), uric acid and peripheral blood leukemia. (b-c) Mean leukemic cell counts±SD over weeks from leukemia challenge in mice receiving CAR-T or CTRL cells are shown. (d-e) Mean percentages of body weight variations±SD over days from CAR-T or CTRL cells are shown. Dashed lines indicate the threshold for severe weight loss (>15%). (f) Mean body temperature variations±SD over days from CAR-T cells or CTRL cells are shown. Dashed lines indicate the threshold for high fever (ΔT>2° C.). (g) Means±SD of human IFN-γ, IL-2, TNF-α, IL-10 or IL-6 serum concentrations measured by cytokine immunoassay 7 days after CAR-T or CTRL cells in n=4 mice with high leukemia burden per group are shown. (h-i) Means±SD of mouse SAA and uric acid serum concentrations over days from CAR-T or CTRL cells in mice with high leukemia burden are shown. Results from a two-way ANOVA test with Bonferroni correction are indicated when statistically significant (*, P<0.05; **, P<0.01; ***, P<0.001).

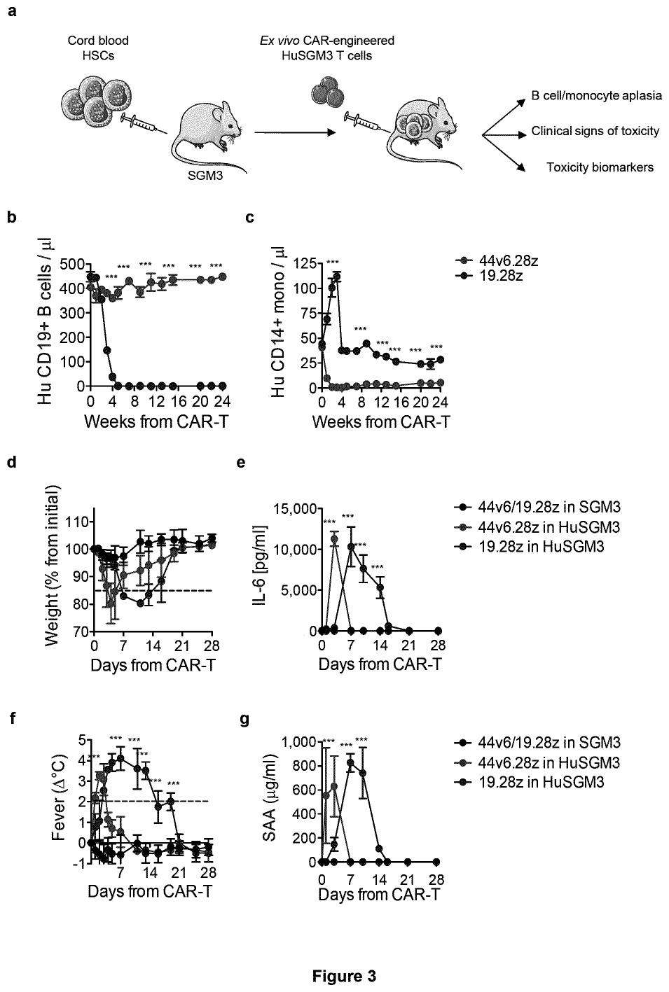

FIG. 3. Non-xenoreactive CAR-T cells induce CRS in HuSGM3 mice. (a) Adult SGM3 mice (8 weeks of age) were infused i.v. with 10⁵ CB HSCs (n=3 donors, HuSGM3) and after 4 weeks with 2×10⁶ T cells from newborn HuSGM3 mice (same HSC donors) that had been ex vivo engineered with either a CD44v6.28z CAR (44v6.28z in HuSGM3, n=15 mice from three independent experiments) or a CD19.28z CAR (19.28z in HuSGM3, n=15 mice). Non HSC-humanized SGM3 mice were infused with either HuSGM3 CD44v6.28z or CD19.28z CAR-T cells as control, and results were pooled (44v6/19.28z in SGM3, n=18 mice). Secondary recipients were followed over time by daily monitoring of weight loss and body temperature and weekly monitoring of serum concentrations of human IL-6 and circulating human B cells/monocytes. (b) Mean counts±SD of human CD19⁺ B cells or (c) CD14⁺ monocytes over days from 44v6.28z or 19.28.z HuSGM3 CAR-T cells are shown. (d) Mean percentages of body weight variations±SD over days from either 44v6/19.28z CAR-T cells in SGM3 mice, or from 44v6.28z or 19.28.z CAR-T cells in HuSGM3 mice are shown. Dashed lines indicate the threshold for severe weight loss (>15%). (e) Mean human IL-6 serum concentrations±SD over days from CAR-T cells are shown. (f) Mean body temperature variations±SD over days from CAR-T cells are shown. Dashed lines indicate the threshold for high fever (ΔT>2° C.). (g) Means±SD of mouse SAA concentrations over days from CAR-T are shown. Results from a two-way ANOVA test with Bonferroni correction are indicated when statistically significant (*, P<0.05; **, P<0.01; ***, P<0.001).

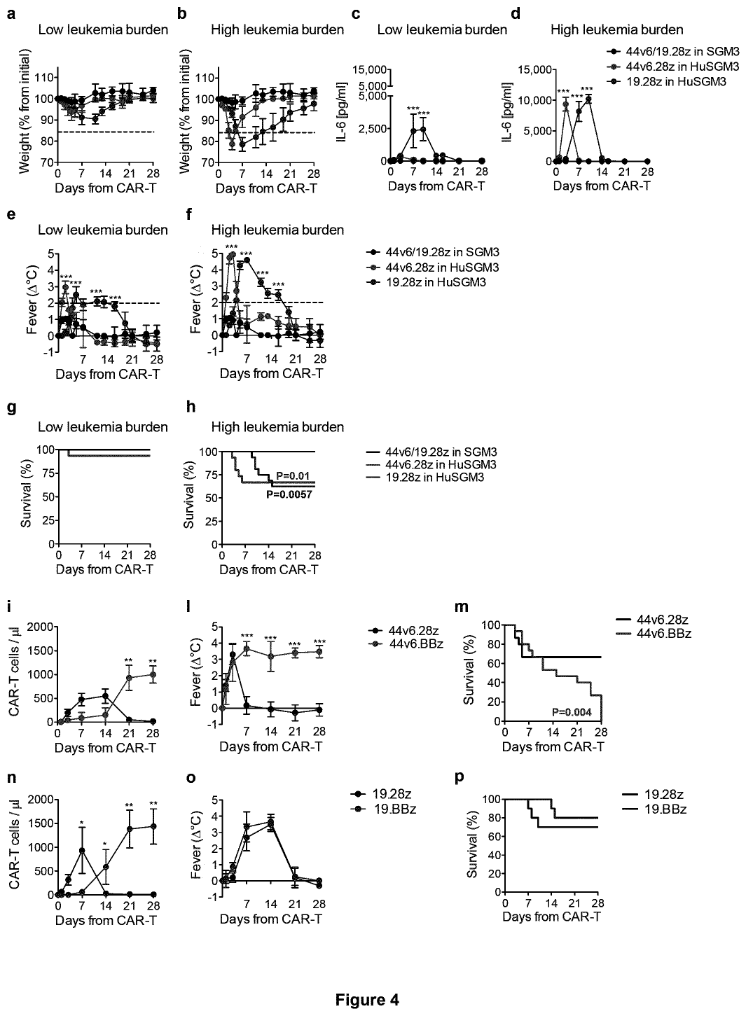

FIG. 4. CRS severity by non-xenoreactive CAR-T cells in HuSGM3 mice correlates with leukemia burden. Adult SGM3 mice (8 weeks of age) were co-infused i.v. with 10⁵ CB HSCs (n=3 donors, HuSGM3) and 5×10⁶ CD19⁺CD44v6⁺ ALL-CM leukemic cells, and, after either 4 weeks (low leukemia burden) or 7 weeks (high leukemia burden), with HuSGM3 T cells (same HSC donors) that had been ex vivo engineered with either a CD44v6.28z CAR (44v6.28z, n=15 mice from three independent experiments) or a CD19.28z CAR (19.28z, n=15 mice). Leukemic non HSC-humanized SGM3 mice were infused with either HuSGM3 CD44v6.28z or CD19.28z CAR-T cells as control, and results were pooled (44v6/19.28z in SGM3, n=18 mice). Secondary recipients were followed over time by daily monitoring of weight loss and body temperature, and weekly monitoring of serum concentrations of human IL-6. (a-b) Mean percentages of body weight variations±SD, (c-d) mean human IL-6 serum concentrations±SD over days from CAR-T cells, (e-f) mean body temperature variations±SD over days from CAR-T cells are shown. Results from a two-way ANOVA test with Bonferroni correction are indicated when statistically significant (*, P<0.05; **, P<0.01; ***, P<0.001). (g-h) Kaplan-Meyer survival plots of leukemic SGM3 mice infused with 44v6/19.28z CAR-T cells or leukemic HuSGM3 mice infused with either 44v6.28z or 19.28.z CAR-T cells are shown (for survival criteria, see Methods). Results from a Mantel-Cox (log-rank) test are indicated as exact P values of 44v6.28z in HuSGM3 vs 44v6/19.28z in SGM3 (red, hazard ratio: 10.3, 1.7-61.3 95% CI) or of 19.28z in HuSGM3 vs 44v6/19.28z in SGM3 (blue, hazard ratio: 9.8, 1.9-49.9 95% CI). CRS mortality was defined as death preceded by high fever (ΔT>2° C.) and human IL-6 serum concentration>1,500 pg/ml. (i) Adult SGM3 mice (8 weeks of age) were co-infused i.v. with 10⁵ CB HSCs (n=3 donors) and 5×10⁶ CD19⁺CD44v6⁺ ALL-CM leukemic cells and, after 7 weeks with HuSGM3 T cells (same HSC donors) that had been ex vivo engineered with either a CD44v6.28z CAR (44v6.28z, n=10 mice from two independent experiments), a CD44v6.BBz CAR (44v6.BBz, n=10), a CD19.28z CAR (19.28z, n=10 mice) or a CD19.BBz CAR (19.BBz, n=10). Mean CAR-T cell counts±SD, (I) mean body temperature variations±SD and (m) Kaplan-Meyer survival plots of HuSGM3 mice infused with CD44v6 CAR-T cells are shown. Results from a Mantel-Cox (log-rank) test are indicated as exact P values of 44v6.BBz vs 44v6.28z (red, hazard ratio: 0.3, 0.1-0.6 95% CI). (n) Mean CAR-T cell counts±SD, (o) mean body temperature variations±SD and (p) Kaplan-Meyer survival plots of HuSGM3 mice infused with CD19 CAR-T cells are shown. Dashed lines indicate the threshold for high fever (ΔT>2° C.). Results from a two-way ANOVA test with Bonferroni correction are indicated when statistically significant (*, P<0.05; **, P<0.01; ***, P<0.001).

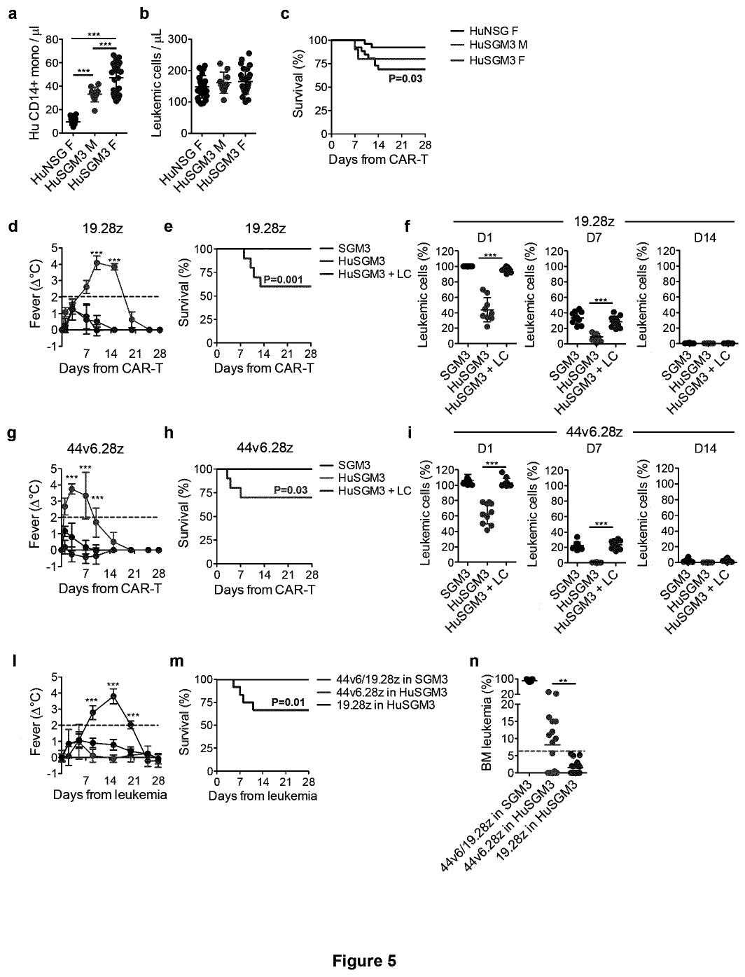

FIG. 5. Circulating monocyte ablation by non-xenoreactive CD44v6 CAR-T cells protects HuNSG-SGM3 mice from CRS. Eight-weeks old female NSG (n=26 from 3 independent experiments, HuNSG F), male SGM3 (n=10, HuSGM3 M) or female SGM3 (n=26, HuSGM3 F) were co-infused i.v. with 10⁵ CB HSCs and 5×10⁶ CD19⁺CD44v6⁺ ALL-CM leukemic cells and, after 5 weeks, with HuSGM3 T cells (same HSC donors) that had been ex vivo engineered with a CD19.28z CAR. Secondary recipients were followed for survival over days from CAR-T cells. (a) Mean counts±SD of human CD14⁺ monocytes, (b) of leukemic cells±SD at 5 weeks prior to CAR-T cells are shown. Results from a one-way ANOVA test with Bonferroni correction are indicated when statistically significant (***, P<0.001). (c) Kaplan-Meyer survival plots of HuNSG F, HuSGM3 M or HuSGM3 F mice infused with CD19.28z CAR-T cells are shown. Results from a Mantel-Cox (log-rank) test are indicated as exact P values of HuSGM3 F vs HuNSG F (blue, hazard ratio: 3.8, 1.1-13.3 95% CI). (d) Leukemic HuSGM3 mice were treated or not with liposomal clodronate (LC, n=20 per group from two independent experiments) prior to the infusion of CD19.28z (n=10) or CD44v6.28z (n=10) CAR-T cells. Non HSC-humanized SGM3 mice were used as control (n=20). (d,g) Mean body temperature variations±SD over days from CAR-T cells are shown. Dashed lines indicate the threshold for high fever (ΔT>2° C.). Results from a two-way ANOVA test with Bonferroni correction are indicated when statistically significant (*, P<0.05; **, P<0.01; ***, P<0.001). (e,h) Kaplan-Meyer survival plots of SGM3, HuSGM3 or HuSGM3+LC mice infused with CD19.28z or (h) CD44v6.28z CAR-T cells are shown. Results from a Mantel-Cox (log-rank) test are indicated as exact P values of HuSGM3+LC vs HuSGM3 (red, hazard ratio: 8.3, 0.9-80.5 95% CI). (f) Mean leukemic cells percentages±SD after one, 7 and 14 days from CD19.28z or (i) CD44v6.28z CAR-T cell infusion are shown. Results from a one-way ANOVA test with Bonferroni correction are indicated when statistically significant (***, P<0.001). (I) HuSGM3 mice were infused with HuSGM3 CD44v6.28z (44v6.28z, n=15 mice from three independent experiments or CD19.28z (19.28z, n=15 mice) and, after 3 weeks, with 5×10⁶ CD19⁺CD44v6⁺ ALL-CM leukemic cells. non HSC-humanized SGM3 mice were infused with either HuSGM3 CD44v6.28z or CD19.28z CAR-T cells as control, and results were pooled (44v6/19.28z in SGM3, n=17 mice). Mean body temperature variations±SD over days from leukemic cells are shown. Dashed lines indicate the threshold for high fever (ΔT>2° C.). Results from a two-way ANOVA test with Bonferroni correction are indicated when statistically significant (*, P<0.05; **, P<0.01; ***, P<0.001). (m) Kaplan-Meyer survival plots of SGM3 mice infused with 44v6/19.28z CAR-T cells or HuSGM3 mice infused with either 44v6.28z or 19.28.z CAR-T cells are shown. Results from a Mantel-Cox (log-rank) test are indicated as exact P values of 19.28z in HuSGM3 vs 28z in SGM3 (blue, hazard ratio: 13.9, 1.8-105.0 95% CI). (n) Mean bone marrow (BM) leukemic cells±SD 24 weeks after CAR-T cell infusion are shown. Results from a one-way ANOVA test with Bonferroni correction are indicated when statistically significant (**, P<0.01).

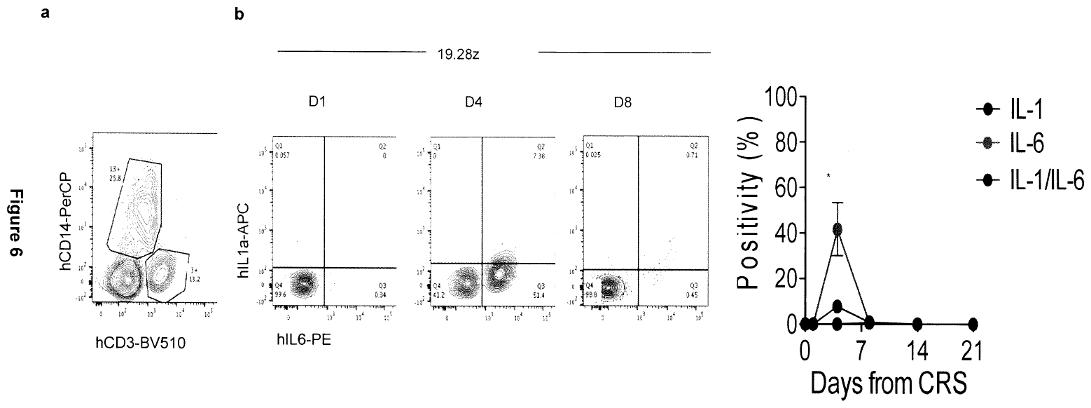

FIG. 6. Monocytic cells are the key cellular sources for IL-6 and IL-1 release upon leukemia recognition by CAR-T cells. Eight-weeks old SGM3 mice (n=8 from 2 independent experiments) were co-infused i.v. with 10⁵ CB HSCs and 5×10⁶ CD19⁺CD44v6⁺ ALL-CM leukemic cells and, after 5 weeks, with HuSGM3 T cells (same HSC donors) that had been ex vivo engineered with a CD19.28z CAR. Secondary recipients were followed over time by daily monitoring of weight loss, body temperature, and intracytoplasmic staining of human IL-1/IL-6 on peripheral blood. (a) Representative plot of human CD3+ CAR-T cells and CD14+ monocytes in leukemic HuSGM3 mice after 7 days from CD19.28z CAR-T cell infusion is shown. (b-c) Representative plots (left) and mean±SD human IL-1/IL-6 production over days from CRS onset by (b) CD19.28z CAR-T cells and (c) monocytes are shown. Results from a two-way ANOVA test with Bonferroni correction are indicated when statistically significant (*, P<0.05; **, P<0.01; ***, P<0.001). (d) tSNE plot incorporating scRNA-Seq data of human CD45+ cells sorted from spleen of leukemic HuSGM3 mice infused with CD19.28z CAR-T cells at day 2 and day 7 of CRS (n=6511). Colors and numbers in the legend indicate transcriptionally defined clusters as well as the assigned cell type based on gene signature analyses. Representative discriminative genes are shown for clusters 5, 7, 11 and 12 (DCs and monocytes). Each dot represents an individual cell. (e) Bar plots showing mean expression (log transformed TPM values, normalized for number of cells) of the indicated genes for each cluster.

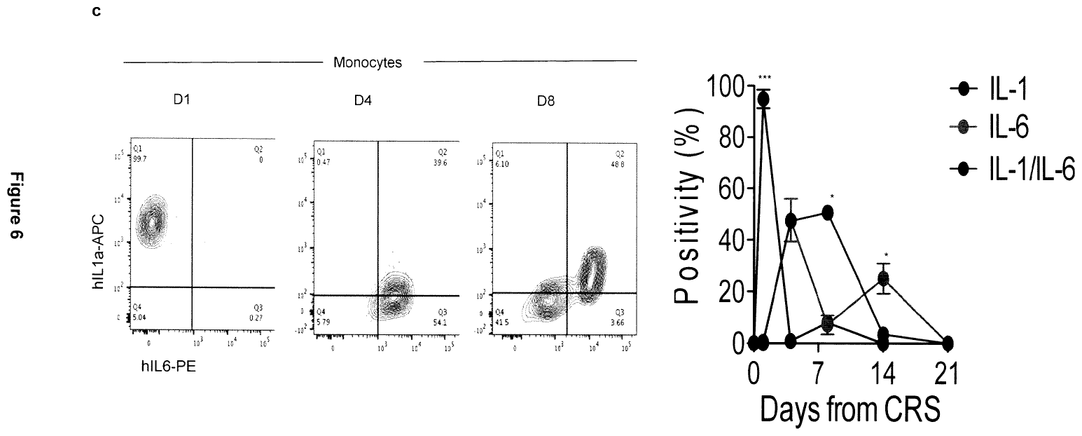

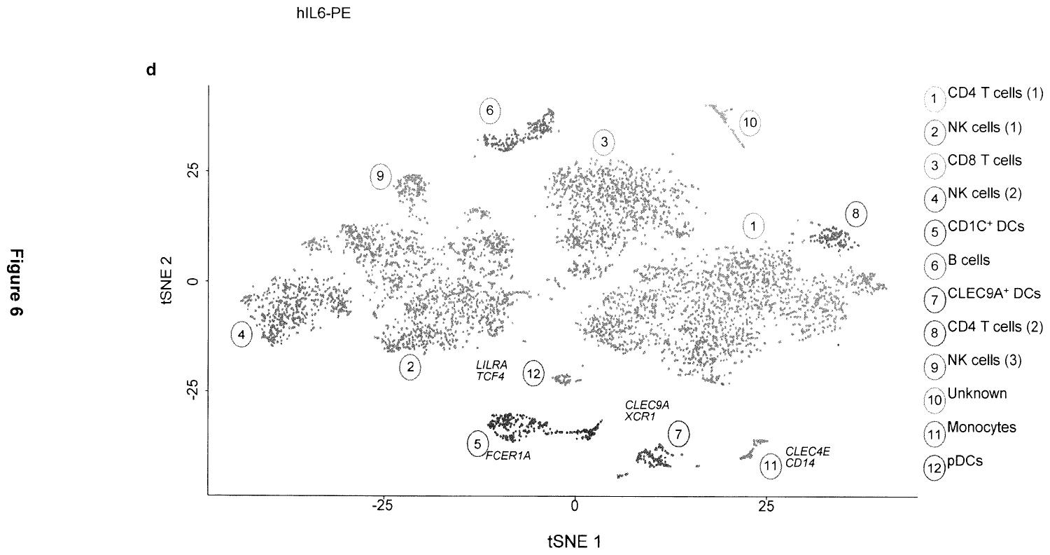

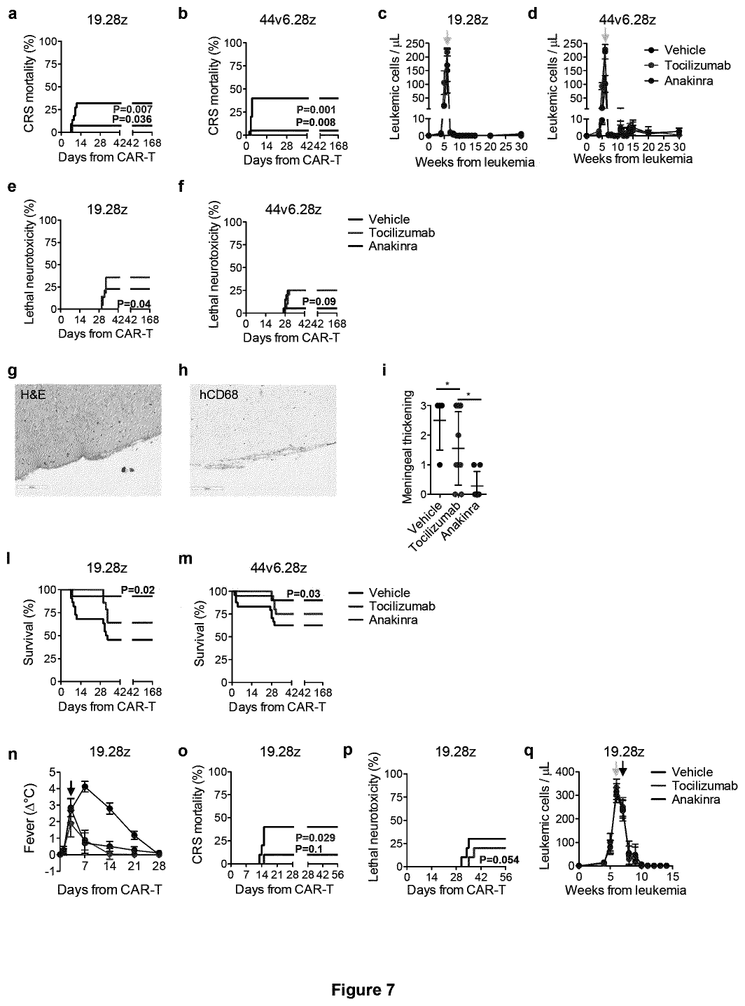

FIG. 7. Anakinra, but not tocilizumab, abolishes neurotoxicity by non-xenoreactive CAR-T cells in HuSGM3 mice. Adult SGM3 mice (8 weeks of age) were co-infused i.v. with 10⁵ CB HSCs (n=3 donors, HuSGM3) and 5×10⁶ CD19⁺CD44v6⁺ ALL-CM leukemic cells, and, after 7 weeks with 2×10⁶ HuSGM3 T cells (same HSC donors) that had been ex vivo engineered with a CD44v6.28z CAR (44v6.28z, n=50 mice from three independent experiments) or a CD19.28z CAR (19.28z, n=50 mice). Just before CAR-T cells, mice received vehicle (n=14 per group), tocilizumab (n=18 per group) or anakinra (n=18 per group) and followed for CRS mortality and lethal neurotoxicity. For doses and schedule of drug administration, see Methods. CRS mortality was defined as death preceded by high fever (ΔT>2° C.) and human IL-6 serum concentration>1,500 pg/ml. Lethal neurotoxicity was defined as death preceded by generalized paralysis or convulsions, in the absence of CRS signs. (a-b) CRS mortality over days from CAR-T cells is shown. Results from a Mantel-Cox (log-rank) test are shown as exact P values comparing tocilizumab (red, hazard ratio: 6.4, 1.6-24.7 95% CI) or anakinra (blue, hazard ratio: 3.9, 1.1-14.4 95% CI) to vehicle in mice infused with 19.28z CAR-T cells, or comparing tocilizumab (red, hazard ratio: 7.9, 2.2-29.2 95% CI) or anakinra (blue, hazard ratio: 5.3, 1.5-18.4 95% CI) to vehicle in mice infused with 44v6.28z CAR-T cells. When non visible, lines are overlapping with x axis. (c-d) Mean leukemic cells counts±SD over weeks from leukemia challenge are shown. Grey arrows indicate CAR-T cell infusion. (e-f) Lethal neurotoxicity over days from CAR-T cells is shown. Results from a Mantel-Cox (log-rank) test are shown as exact P values comparing anakinra (blue, hazard ratio: 6.3, 1.1-37.1 95% CI) to vehicle in mice infused with 19.28z CAR-T cells, or comparing anakinra (blue, hazard ratio: 4.0, 0.8-20.4 95% CI) to vehicle in mice infused with 44v6.28z CAR-T cells. When non visible, lines are overlapping with x axis. (g) Histology (hematoxylin and eosin, H&E) and (h) human CD68 immunohistochemistry pictures of HuSGM3 brain (vehicle) at the time of neurotoxicity are shown. (i) Meningeal thickening quantification (0-3 score, see Methods) in HuSGM3 mice receiving vehicle (n=4), tocilizumab (n=9) or anakinra (n=7) is shown. Results from a two-tailed Mann-Whitney test are indicated when statistically significant (*, P<0.05; **, P<0.01; ***, P<0.001). (l-m) Overall survival over days from CAR-T cells is shown. Results from a Mantel-Cox (log-rank) test are shown as exact P values comparing anakinra (blue, hazard ratio: 3.9, 1.2-12.7 95% CI) to vehicle in mice infused with 19.28z CAR-T cells, or comparing anakinra (blue, hazard ratio: 3.5, 1.0-11.7 95% CI) to vehicle in mice infused with 44v6.28z CAR-T cells. (n-q) Adult SGM3 mice (8 weeks of age) were co-infused i.v. with 10⁵ CB HSCs (n=3 donors, HuSGM3) and 5×10⁶ CD19⁺CD44v6⁺ ALL-CM leukemic cells, and, after 7 weeks with 5×10⁶ HuSGM3 T cells (same HSC donors) that had been ex vivo engineered with a CD19.28z CAR (19.28z, n=20 mice). At the onset of CRS symptomps, mice received vehicle (n=5 per group), tocilizumab (n=7 per group) or anakinra (n=8 per group) and followed for CRS mortality and lethal neurotoxicity. For doses and schedule of drug administration, see Methods. CRS mortality and neurotoxicity were defined above. (n) Mean body temperature variations±SD over days from CAR-T cell infusion are shown. Black arrow indicates beginning of tocilizumab/anakinra treatment. (o) CRS mortality over days from CAR-T cells is shown. Results from a Mantel-Cox (log-rank) test are shown as exact P values comparing tocilizumab or anakinra to vehicle in mice infused with 19.28z CAR-T cells. When non visible, lines are overlapping with x axis. (p) Lethal neurotoxicity over days from CAR-T cells is shown. Results from a Mantel-Cox (log-rank) test are shown as exact P values comparing anakinra to vehicle in mice infused with 19.28z CAR-T cells. When non visible, lines are overlapping with x axis. (q) Mean leukemic cells counts±SD over weeks from leukemia challenge are shown. Grey arrow indicates CAR-T cell infusion. Black arrow indicates beginning of tocilizumab/anakinra treatment.

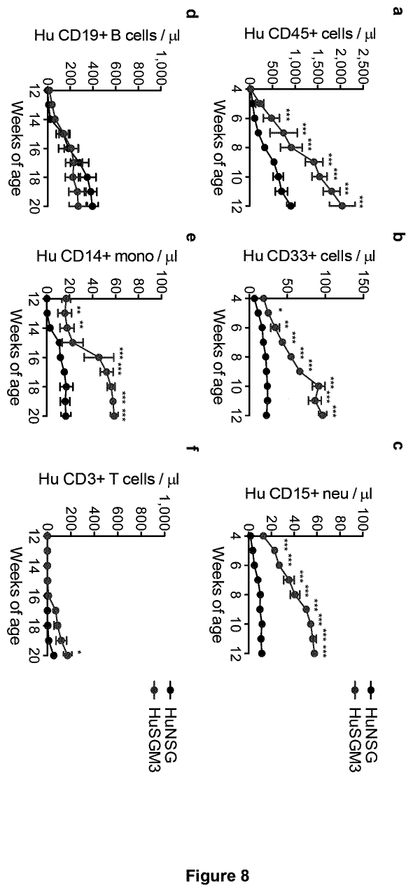

FIG. 8: Human lympho-hematopoietic reconstitution in HuSGM3 mice. (a) Mean counts±SD of circulating human (Hu) CD45+ cells, (b) CD33+ myeloid cells and (c) CD15+ granulocytes in mice over time (weeks of age) are shown. (d) Mean counts±SD of circulating human (Hu) CD19+ B cells, (e) CD14+ monocytes and (f) CD3+ T cells from mice over time (weeks of age) are shown. Data representative of five donors. Results from a two-way ANOVA test are indicated when statistically significant (*, P<0.05; **, P<0.01; ***, P<0.001).

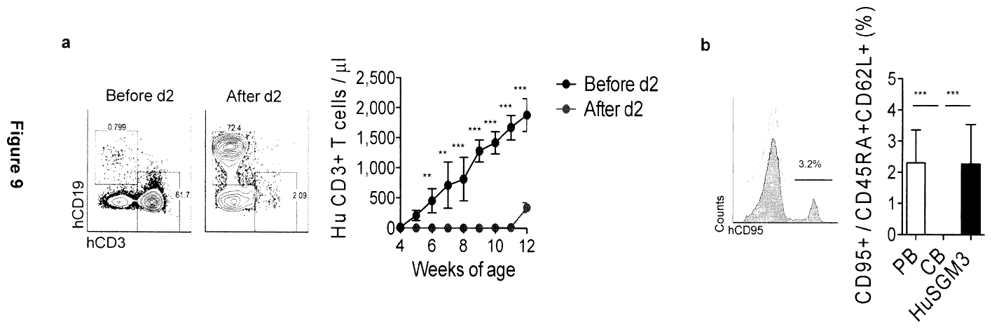

FIG. 9: Human T cell repopulation of lymphoid organs in HuSGM3 mice. (a) Representative plot of circulating human CD3 T cells in HuSGM3 mice at 5 weeks of age (left) and mean CD3 counts±SD in HuSGM3 mice transplanted before (n=10) or after (n=10) post-natal day 2 are shown. Results from a one- or a two-way ANOVA test are indicated when statistically significant (**, P<0.01; ***, P<0.001). (b) Representative plot of circulating human CD95+ on CD8+/CD45RA+/CD62L+ T cells at 12 weeks of age (left) and mean counts±SD in human peripheral blood (PB, n=16 donors), CB (n=12 donors) and HuSGM3 T cells (right) are shown. Staining with isotype control antibody is shown in grey and specific antibody in red. (c) Representative plot of circulating human CD3 T cells in HuSGM3 mice at 5 weeks of age (left) and mean CD3 counts±SD in HuSGM3 mice in the thymus, (d) spleen and (e) bone marrow are shown. Results from Mann-Whitney test are indicated when statistically significant (***P<0.001).

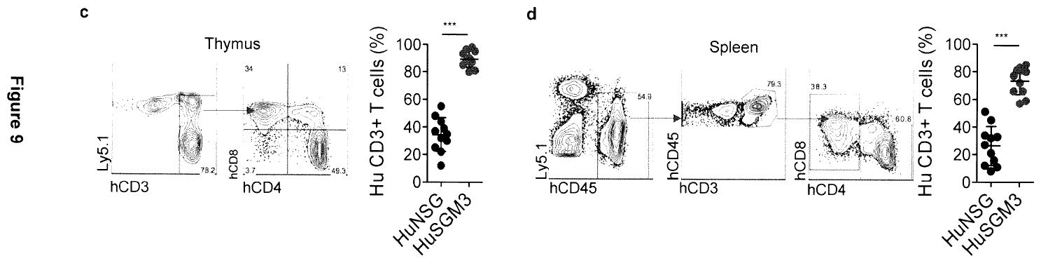

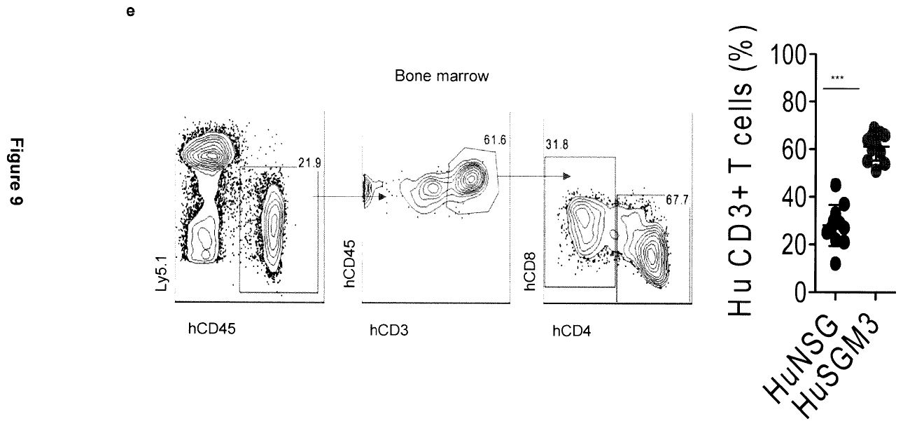

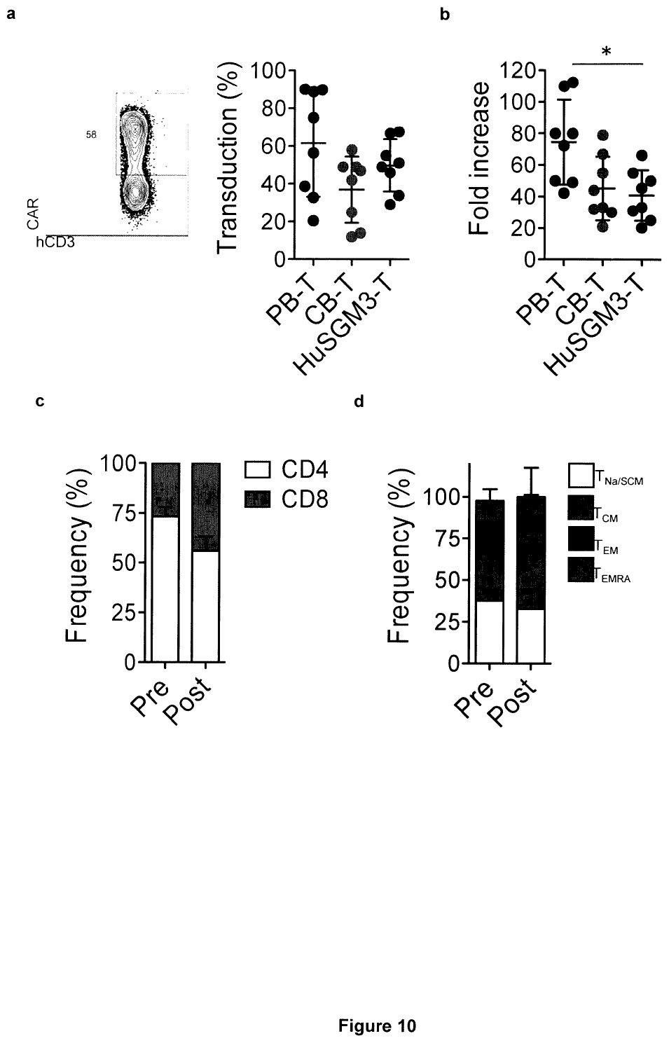

FIG. 10: CAR engineering of HuSGM3 T cells. (a) Representative plot (left) and frequencies±SD (right) of transduction efficiency, (b) fold increase frequencies±SD of CAR-T cells in human peripheral blood (PB, n=8 donors), CB (n=8 donors) and HuSGM3 (n=8) 15 days after activation. (c) mean human CD4/CD8 frequencies±SD and (d) mean human T_{Na} (CD45RA+/CD62L+), T_{CM} (CD45RA−/CD62L+), T_{EM} (CD45RA−/CD62L−) and T_{EMRA} (CD45RA+CD62L−) frequencies in HuSGM3 CAR-T cells before (Pre, n=8) and after (Post, n=8) ex vivo activation are shown. Results from one-way ANOVA test and Bonferroni correction are indicated when statistically significant (*P<0.05).

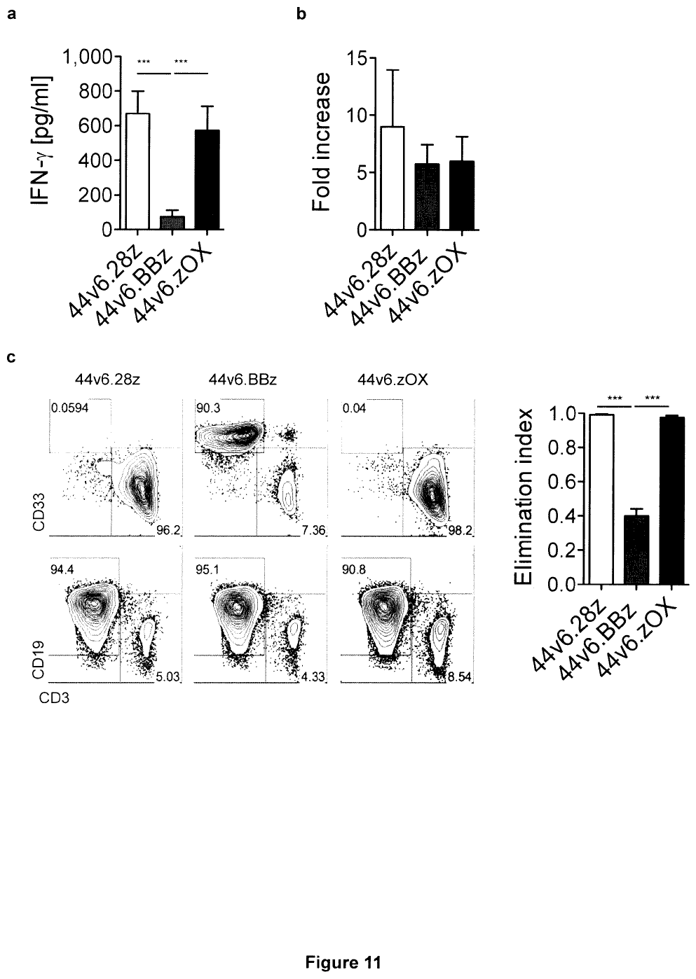

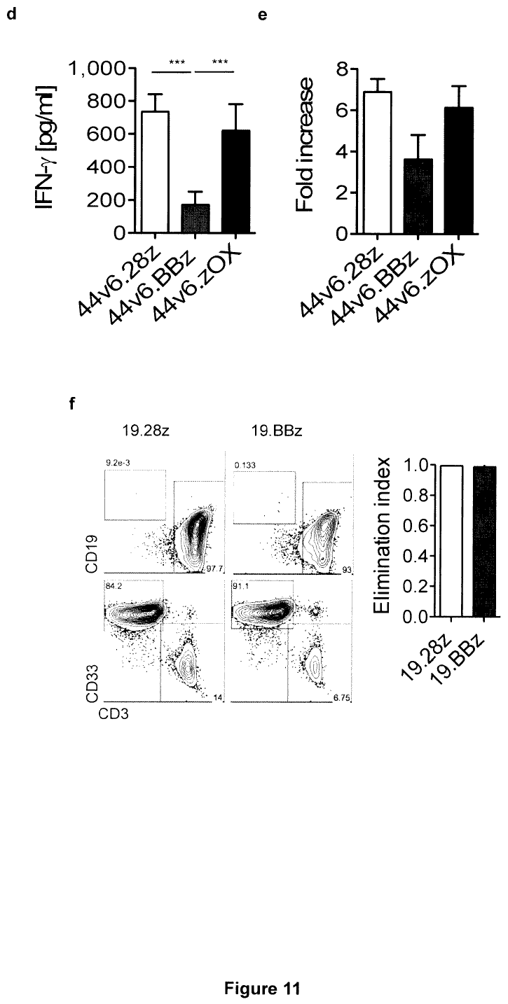

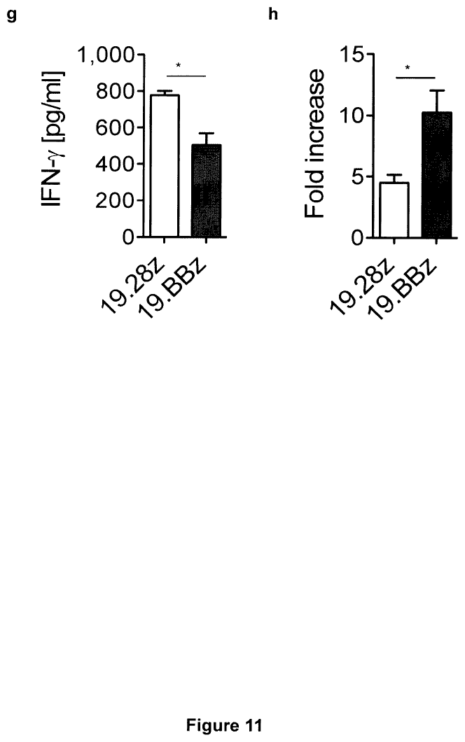

FIG. 11: In vitro functionality of HuSGM3 CAR-T cells. (a) HuSGM3 T cells were activated with CD3/CD28-beads and IL-7/IL-15, and RV transduced with either a CD44v6.28z, a CD44v6.BBz or a CD44v6.zOx CAR (see Methods). Mean IFN-g production±SD in HuSGM3 CAR-T cells in response to CD44v6⁺ leukemic cells after one day of co-culture are shown. (b) fold increase frequencies±SD of CAR-T cells after 4-days co-culture are shown. (c) PB-T cells were activated with CD3/CD28-beads and IL-7/IL-15, and RV transduced with either a CD44v6.28z, a CD44v6.BBz or a CD44v6.zOx CAR (see Methods). PB CAR-T cells were co-cultured at a 1:10 E:T ratio with CD33⁺CD44v6⁺ THP-1 leukemic cells (upper row) or with CD19⁺/CD44v6⁻ BV173 leukemic cells (lower row). Representative plots after 4-days co-culture (left) and mean elimination indexes±SD by CD44v6 CAR-T cells of different design from 5 independent experiments are shown (right). (d) Mean IFN-g production±SD in PB CAR-T cells in response to CD44v6⁺ leukemic cells after one day of co-culture are shown. (e) fold increase frequencies±SD of CAR-T cells after 4-days co-culture are shown. (f) HuSGM3 T cells were activated with CD3/CD28-beads and IL-7/IL-15, and RV transduced with either a CD19.28z or a CD19.BBz CAR (see Methods). CAR-T cells were co-cultured at a 1:10 E:T ratio with CD19⁺ BV-173 leukemic cells (upper row) or with CD33⁺/CD19⁻ THP-1 leukemic cells (lower row). Representative plots after 4-days co-culture (left) and mean elimination indexes±SD by CD19 CAR-T cells of different design from 5 independent experiments are shown (right). (g) Mean IFN-g production±SD in HuSGM3 CAR-T cells in response to CD19⁺ leukemic cells after one day of co-culture are shown. (e) fold increase frequencies±SD of HuSGM3 CAR-T cells after 4-days co-culture are shown. Results from Mann-Whitney test with Bonferroni correction are shown when statistically significant (*, P<0.05; ***, P<0.001).

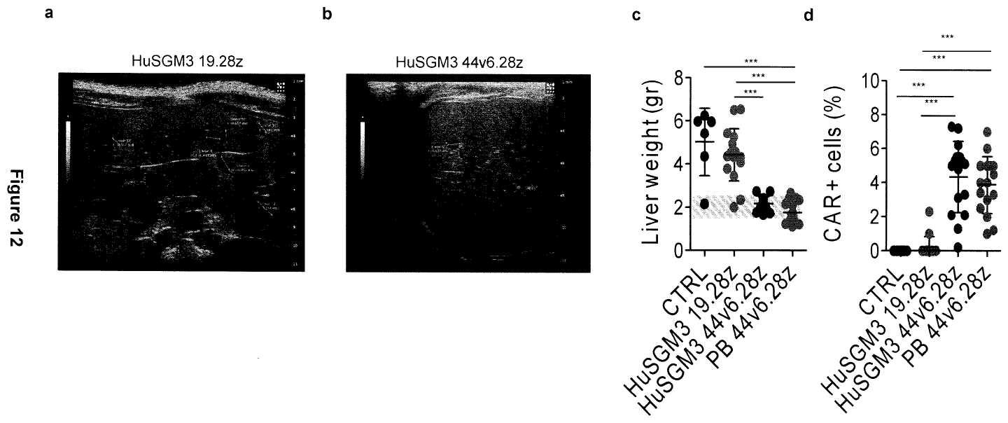

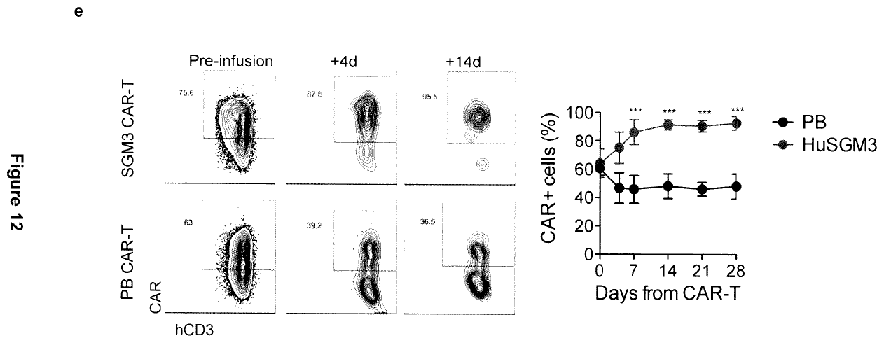

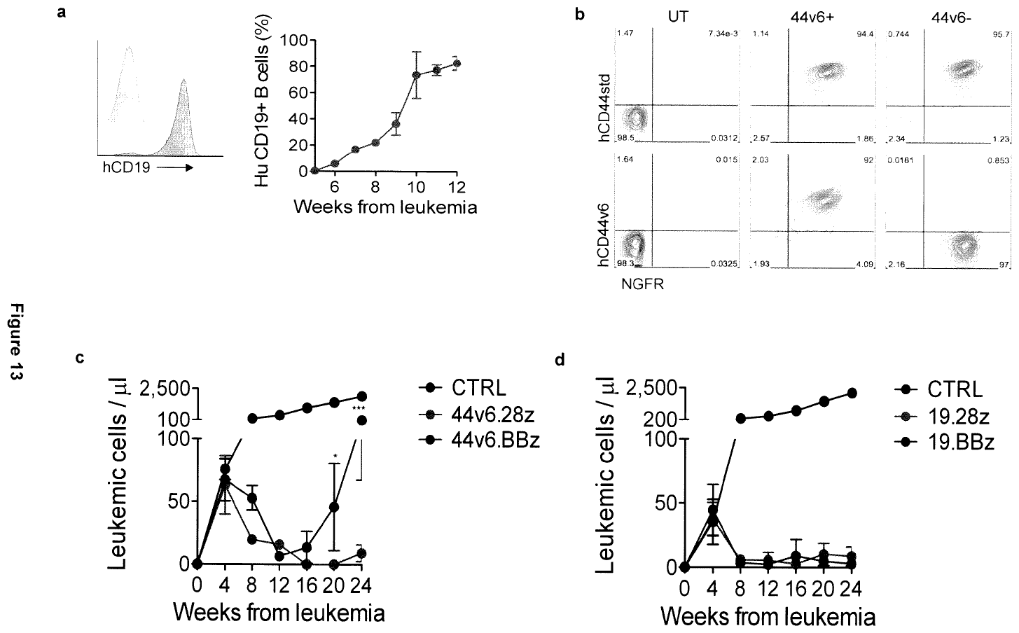

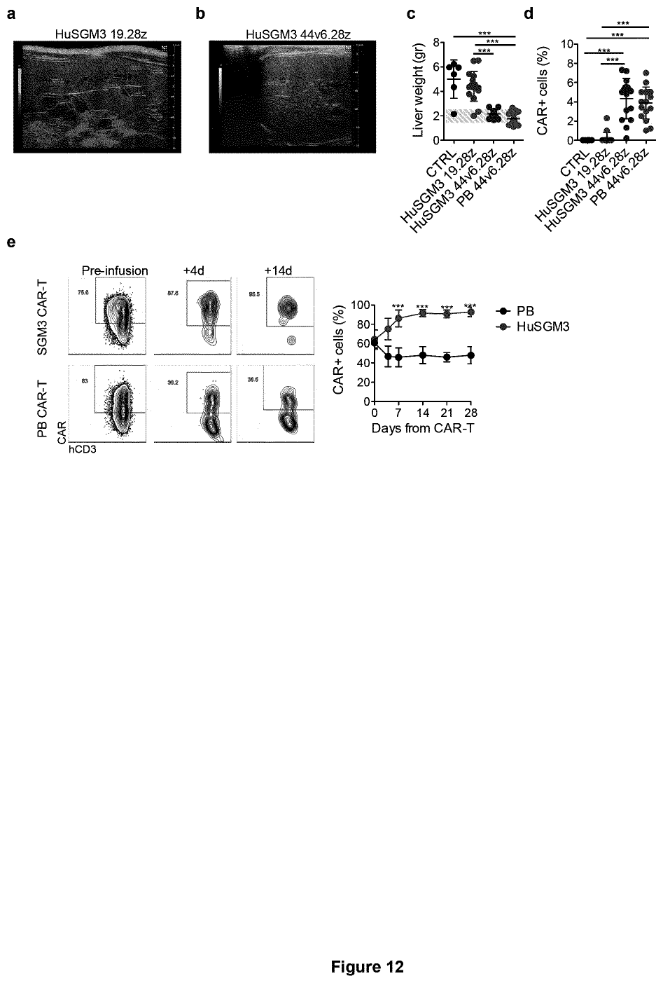

FIG. 12: In vivo antileukemic effects by HuSGM3 CAR-T cells. (a) NSG mice were engrafted with CD19⁻/CD44v6⁺ THP-1 leukemic cells and,after one week, with 5×10⁶ CD44v6 CAR-T cells from HuSGM3 (n=16 from 3 independent experiments), PB (n=16 from 3 independent experiments), or with CTRL CD19 CAR-T cells from HuSGM3 (n=16 from 3 independent experiments). THP-1 leukemic cell progression by hepatic echography after 28 days in CTRL (HuSGM3 19.28z) or (b) CD44v6 (HuSGM3 44v6.28z) CAR-T cells are shown.(c) Antileukemia efficacy after 35 days by liver weight analysis is shown. Results from a one-way ANOVA test with Bonferroni correction are shown when statistically significant (***P<0.001). (d) Representative plots (left) and mean CAR⁺ cell frequencies±SD are shown. Results from a two-way ANOVA test with Bonferroni correction are shown when statistically significant (***P<0.001).

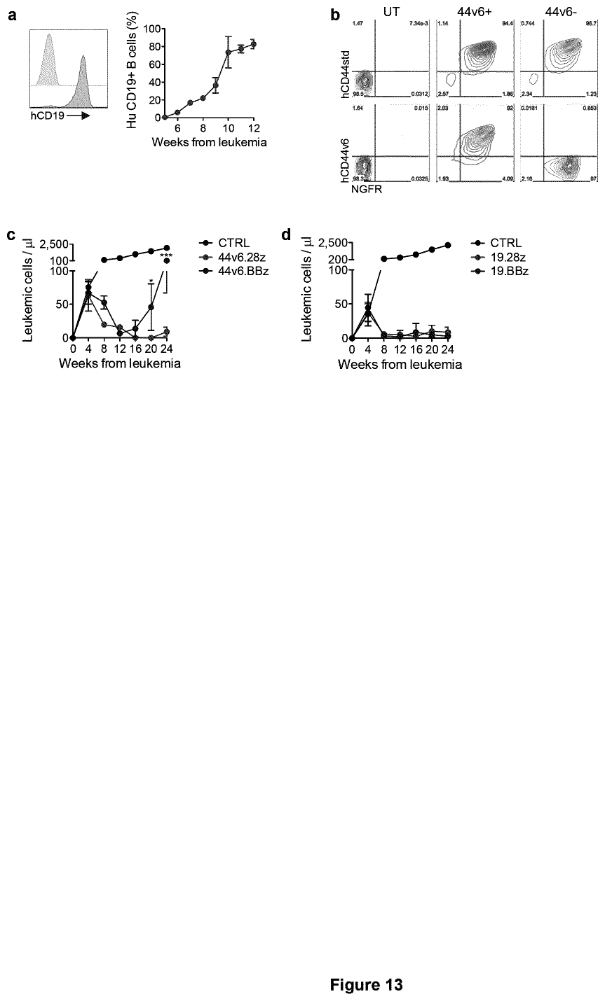

FIG. 13: Suboptimal antileukemia efficacy by CD44v6.BBz HuSGM3 CAR-T cells. (a) Representative plot (left) and mean CD19⁺ ALL-CM leukemic cell frequencies±SD in NSG mice (n=8) are shown. (b) Representative plots of CD44std⁻/CD44v6⁻/NGFR⁻ ALL-CM untransduced (UT, left), CD44std⁺/CD44v6⁺/NGFR⁺ ALL-CM (44v6⁺, middle) and CD44std⁺/CD44v6⁺/NGFR⁺ (44v6⁻, right) cells are shown. (c) Adult SGM3 mice were infused i.v. with CD19⁺/CD44v6⁺ ALL-CM leukemic cells and after 5 weeks with 5×10⁶ T cells from newborn HuSGM3 mice (n=3 HSC donors) that had been ex vivo engineered with either a CD44v6.28z (n=6), CD44v6.BBz (n=6), CD19.28z (n=6) or CD19.BBz (n=6) CAR or left untransduced (CTRL, n=6). Mean leukemic cell counts±SD over weeks from tumor challenge are shown. Results from a two-way ANOVA test with Bonferroni correction are shown when statistically significant (*, P<0.05; ***, P<0.001).

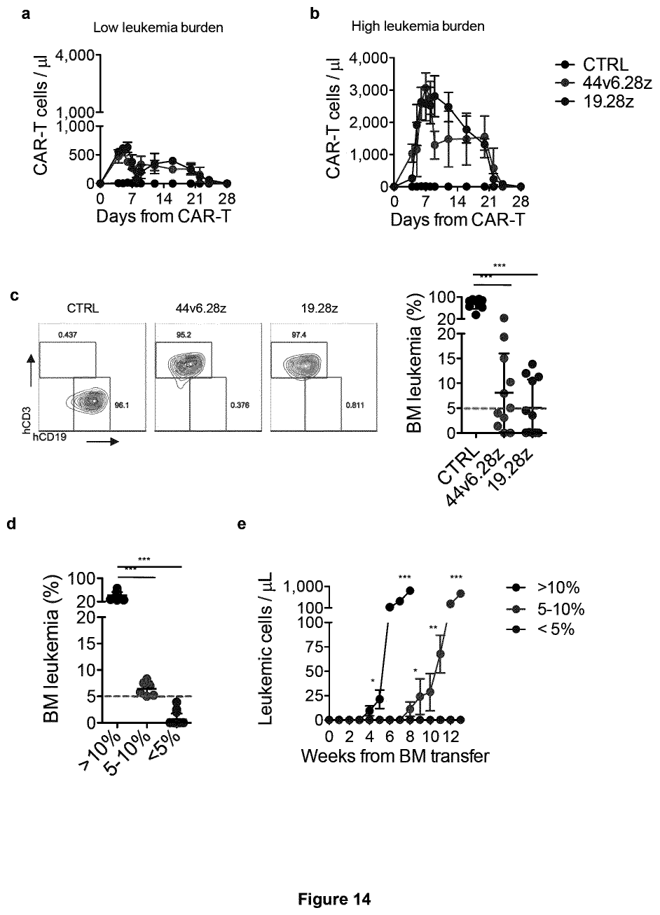

FIG. 14: Deep leukemia remissions by HuSGM3 CAR-T cells. Adult SGM3 mice were infused i.v. with CD19⁺/CD44v6⁺ ALL-CM leukemic cells and after 5 weeks with 2×10⁶ T cells from newborn HuSGM3 mice (n=3 HSC donors) that had been ex vivo engineered with either a CD44v6.28z CAR (n=12 from 2 independent experiments) or a CD19.28z CAR (n=12 from 2 independent experiments) or left untransduced (CTRL, n=10). (a-b) HuSGM3 CAR-T cell expansion kinetics in low (a) and high (b) tumor burden settings are shown as mean±SD. (c) Representative plots after 24 weeks (left) and mean±SD frequencies of BM leukemic cells in animals are shown. Threshold for minimal residual disease (MRD) identification is set at 5%. Results from one-way ANOVA test with Bonferroni correction are indicated when statistically significant (***P<0.001). Mean±SD frequencies of BM leukemic cells are shown. (d) BM leukemic cells were purified from T cells and injected in SGM3 tertiary recipients. (e) Mean frequencies±SD of circulating leukemic cells in SGM3 recipients (>10%, n=10 from 2 independent experiments; 5-10%, n=6 from 2 independent experiments; <5%, n=10 from 2 independent experiments) are shown. Results from a two-way ANOVA test with Bonferroni correction are indicated when statistically significant. (*, P<0.05; **, P<0.01; ***, P<0.001).

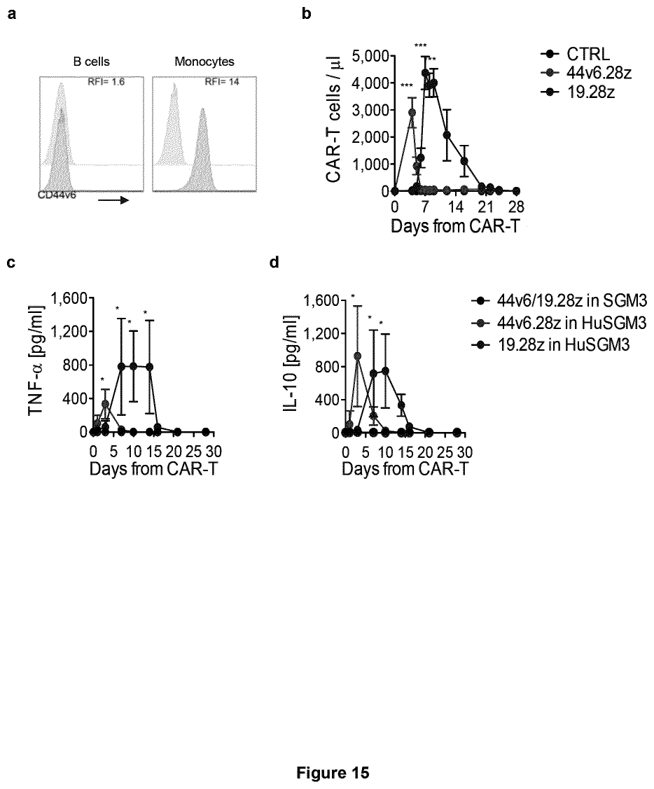

FIG. 15: CRS biomarkers in HuSGM3 mice infused with CAR-T cells (a) Adult SGM3 mice were infused i.v. with 10⁵ CB HSCs (n=3 donors, HuSGM3) and after 4 weeks with 2×10⁶ T cells from newborn HuSGM3 mice (same HSC donors) that had been ex vivo engineered with either a CD44v6.28z (n=15 from 3 independent experiments) or CD19.28z (n=15 from 3 independent experiments). Non HSC-humanized SGM3 mice were infused with either HuSGM3 CD44v6.28z or CD19.28z CAR-T cells as control, and results were pooled (44v6/19.28z in SGM3, n=18 mice). (a) Representative plots for CD44v6 expression on B cells and monocytes from HuSGM3 mice are shown in red. Grey histograms represent isotype control. (b) HuSGM3 CAR-T cell expansion kinetics are shown as mean±SD. (c-d) Mean production±SD of human TNF-a (c) and IL-10 (d) over days from CAR-T cells are shown. Results from a two-way ANOVA test with Bonferroni correction are indicated when statistically significant. (*, P<0.05; ***, P<0.001).

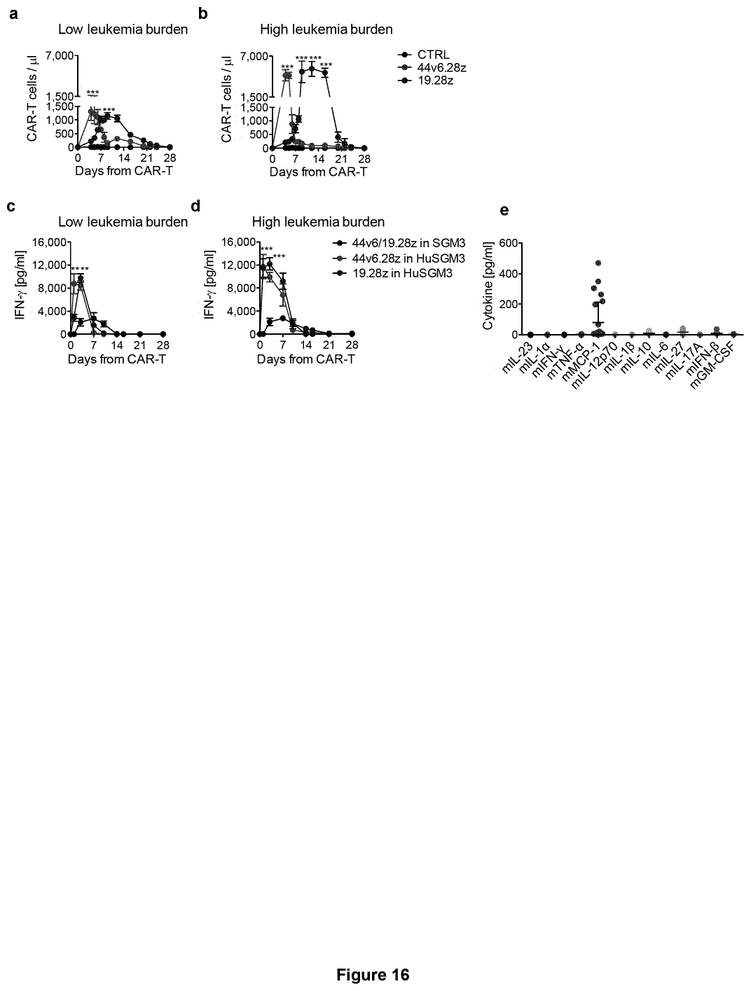

FIG. 16: CAR-T cell expansion levels in HuSGM3 mice with different leukemia burdens. (a-b) Adult SGM3 mice were co-infused i.v. with 10⁵ CB HSCs (n=3 donors, HuSGM3) and 5×10⁶ CD19+CD44v6+ ALL-CM leukemic cells, and, after either 4 weeks (Low leukemia burden) or 7 weeks (High leukemia burden) with HuSGM3 CD44v6.28z (n=15) or CD19.28z (n=15) CAR-T cells. Non HSC-humanized SGM3 mice were infused with either HuSGM3 CD44v6.28z or CD19.28z CAR-T cells as control, and results were pooled (44v6/19.28z in SGM3, n=18). HuSGM3 CAR-T cell expansion kinetics are shown as mean±SD. (c-d) Mean human IFN-g serum concentrations±SD over days from CAR-T cells are shown. Results from a two-way ANOVA test with Bonferroni correction are indicated when statistically significant. (***, P<0.001). (e) Mean human mouse cytokine concentrations±SD at the peak of CRS are depicted. Results from a one-way ANOVA test with Bonferroni correction are indicated when statistically significant. (***, P<0.001).

FIG. 17: Lack of CRS in leukemic HuSGM3 mice infused with irrelevant EGFR.28z CART cells. (a) Adult SGM3 mice were co-infused i.v. with 10⁵ CB HSCs (n=2 donors, HuSGM3) and 5×10⁶ CD19+CD44v6+ ALL-CM leukemic cells, and, after 7 weeks (High leukemia burden) with 2×10⁶ HuSGM3 EGFR.28z CAR-T cells (EGFR.28z, n=6 from two independent experiments). Mean percentages of body weight variations±SD over days are shown. (b) Means±SD of human IL-6 serum concentrations and (c) mean body temperature variations±SD over days are shown. (d) HuSGM3 CAR-T cell expansion kinetics are shown as mean±SD. (e) CRS-free survival and (f) leukemia-free survival over days from CAR-T cells are shown.

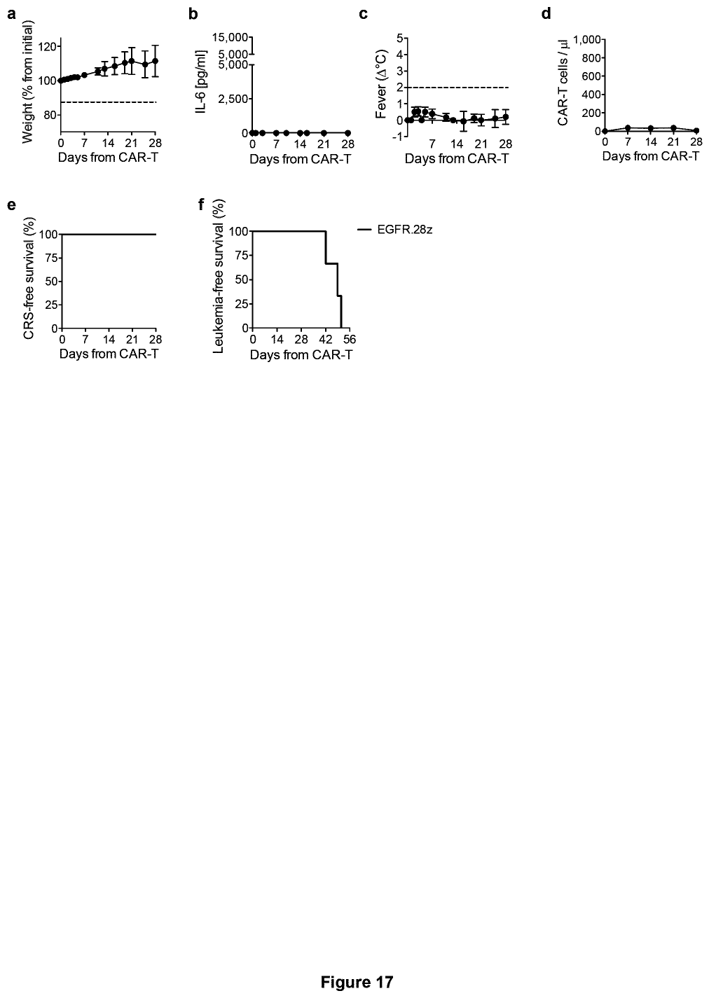

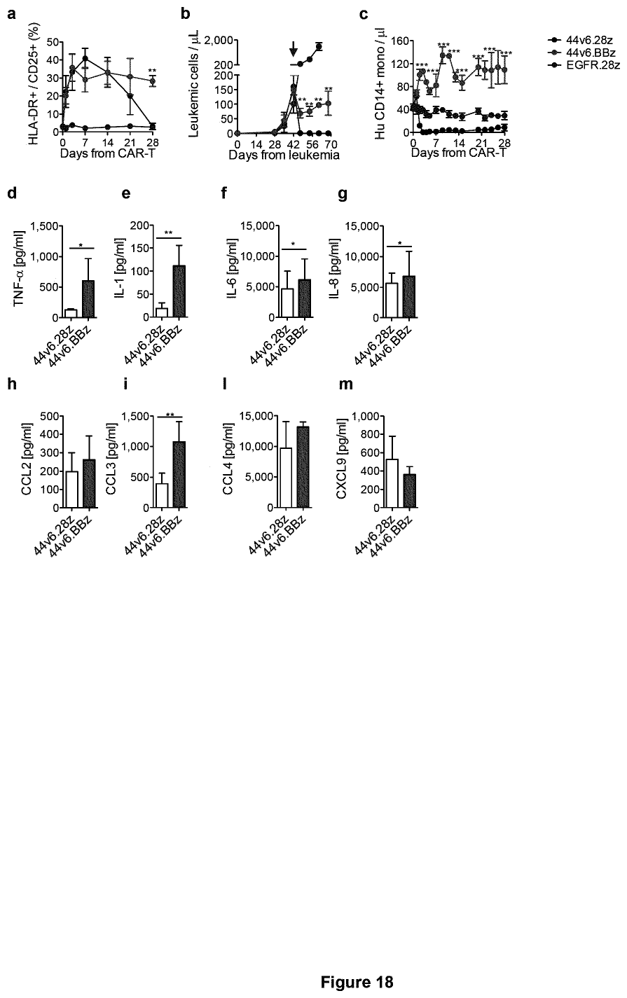

FIG. 18: Monocyte increase in leukemic HuSGM3 mice infused with 44v6.BBz CAR-T cells. (a) Adult SGM3 mice were co-infused i.v. with 10⁵ CB HSCs (n=3 donors) and 5×10⁶ CD19+CD44v6+ ALL-CM leukemic cells and, after 7 weeks with HuSGM3 T cells (same HSC donors) that had been ex vivo engineered with either a CD44v6.28z CAR (44v6.28z, n=10 mice from two independent experiments), a CD44v6.BBz CAR (44v6.BBz, n=10), or control EGFR.28z CAR (EGFR.28z, n=6 from two independent experiments). Mean HLA-DR/CD25+ percentages±SD on CAR-T cells, (b) mean counts±SD of circulating leukemic cells and (c) human monocytes over days are depicted. Results from a two-way ANOVA test with Bonferroni correction are indicated when statistically significant. (**, P<0.01; ***, P<0.001). (d-m) Mean human TNF-a, IL1, IL-6, IL8, CCL2, CCL3, CCL4 and CXCL9 serum concentrations±SD at the peak of CRS are shown. Results from Mann-Whitney test with Bonferroni correction are shown when statistically significant (*, P<0.05; **, P<0.01).

FIG. 19: CAR-T cell expansions in monocyte-depleted HuSGM3 mice. (a) HuSGM3 CD19.28z CAR-T cell expansion kinetics in leukemic HuNSG female, HuSGM3 male or female mice are shown as mean±SD. (b) Mean counts±SD of circulating human monocytes, (c) human B cells and (d) leukemic cells before (pre) and after (post) liposomal clodronate administration are shown. Results from Mann-Whitney test with Bonferroni correction are shown when statistically significant (***, P<0.001). (f) HuSGM3 CD19.28z CAR-T cell expansion kinetics in leukemic HuSGM3 mice infused with liposomal clodronate are shown as mean±SD. (g) Representative plots after 4-days co-culture (left) and mean elimination indexes±SD by CD19 CAR-T cells in the presence or absence of monocytes from 5 independent experiments are shown (right). Results from Mann-Whitney test with Bonferroni correction are shown when statistically significant (**, P<0.01). (h) Mean human IL-6 serum concentrations±SD over time from leukemia challenge are depicted. (i) HuSGM3 CAR-T cell expansion kinetics in prophylactically infused HuSGM3 mice are shown. Arrow indicates leukemia challenge. Results from a two-way ANOVA test with Bonferroni correction are indicated when statistically significant. (**, P<0.01; ***, P<0.001).

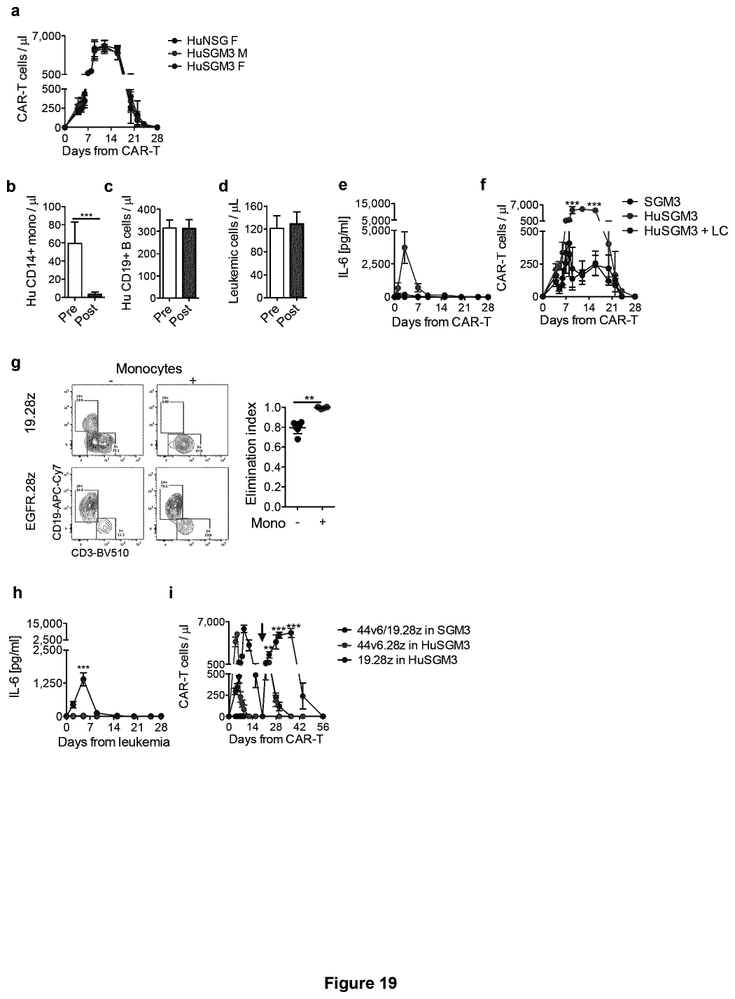

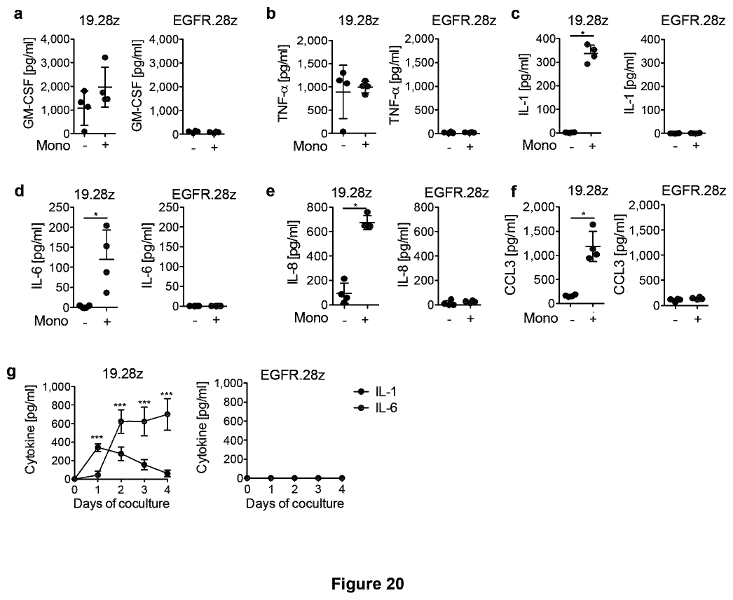

FIG. 20: Monocytic cells are required for IL-6 and IL-1 release upon leukemia recognition by CAR-T cells. T cells from human peripheral blood (n=4 donors) were engineered with a CD19.28z CAR and co-cultured with CD19⁺ ALL-CM leukemic cells. After 48 hrs, supernatants were collected and added to PMA-stimulated THP-1 cells. (a) GM-CSF, (b) TNF-a, (c) IL-8, (d) MIP-1a, (e) IL-1b and (f) IL-6 release was measured by cytokine immunoarray after 24 hrs and is expressed as means±SD. Results from a Student's t-test are shown when statistically significant (*, P<0.05). (g) Time-course analysis of IL-1 and IL-6 release from THP-cell exposed to CAR-T cell supernatants is shown. Results from a two-way ANOVA are depicted when statistically significant (*, P<0.05; **, P<0.01; ***, P<0.001).

FIG. 21: IL-1 and IL-6 production in three-party co-cultures. T cells from human peripheral blood (n=3 donors) were engineered with a CD19.28z CAR and co-cultured with CD19⁺ ALL-CM leukemic cells in presence or absence of autologous monocytes. After 12, 24, or 48 hrs cells were stained for intracytoplasmic detection of human IL-1/IL-6. (a) Representative plot of three-party coculture. (b-d) Representative plots (left) and mean percentages±SD (right) of IL-1/IL6 production after 12, 24 and 48 hrs coculture by CD19.28z CAR-T cells, (c) leukemic cells and (d) monocytes. Results from a two-way ANOVA are depicted when statistically significant (*, P<0.05; **, P<0.01).

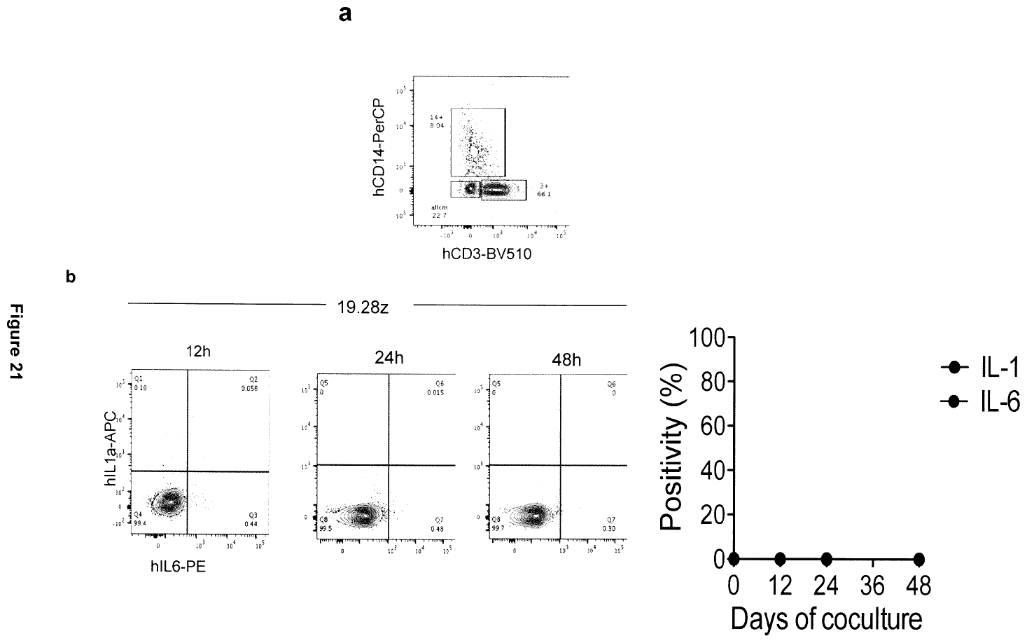

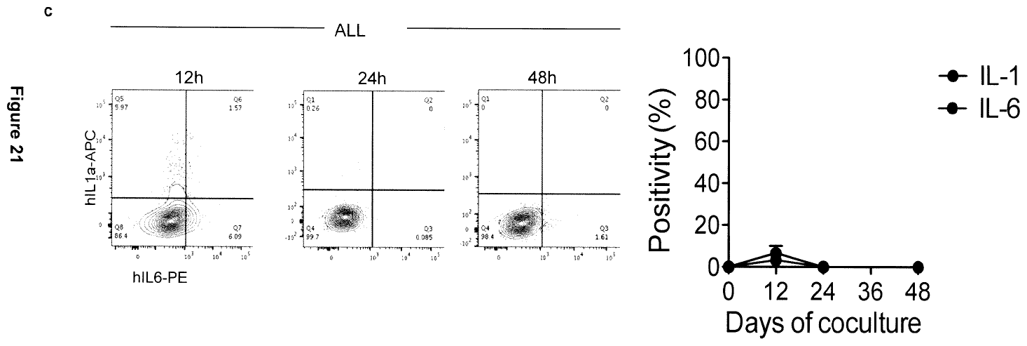

FIG. 22. IL-1 and IL-6 production in leukemic HuSGM3 mice infused with irrelevant EGFR.28z CAR-T cells. T cells from human peripheral blood (n=3 donors) were engineered with a EGFR.28z CAR and co-cultured with CD19⁺ ALL-CM leukemic cells in presence or absence of autologous monocytes. After 12, 24, or 48 hrs cells were stained for intracytoplasmic detection of human IL-1/IL-6. (a-b) Representative plots (left) and mean percentages±SD (right) of IL-1/IL6 production after 12, 24 and 48 hrs coculture by EGFR.28z CAR-T cells and (b) monocytes. (c) Representative plots (left) and mean±SD (right) of in vivo human IL-1/IL-6 production by CD4 and CD8 CD19-28z CAR-T cells in leukemic HuSGM3 mice. Results from a Student's t-test are shown when statistically significant (***, P<0.001).

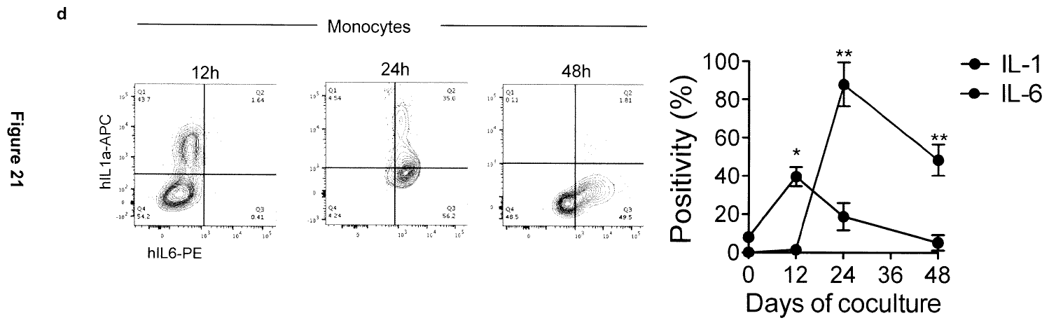

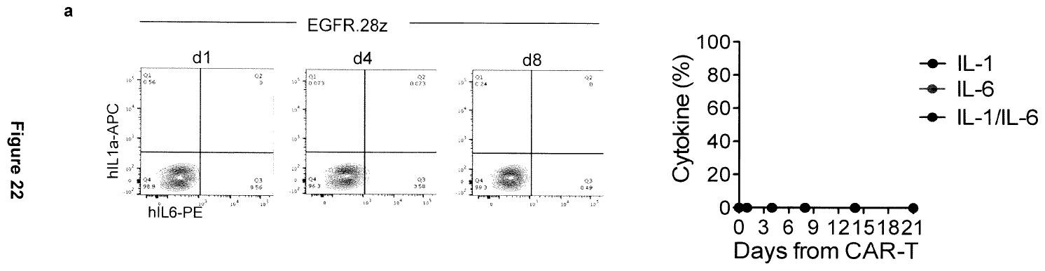

FIG. 23: Definition of human lymphoid and myeloid cell populations in HuSGM3 in CRS by scRNA-Seq. (a-b) Correlation analyses of replicate scRNA-Seq experiments, showing mean gene expression values in the indicated conditions. (c-f) tSNE plots showing single-cell gene expression levels of a T-cell signature (CD3D, CD3E, CD3G, CD27, CD28), (d) CD8/CD4, (e) B-cell (CD19, MS4A1, CD79A, CD79B, BLNK) and (f) NK-cell signature (FCGR3A, FCGR3B, NCAM1, KLRB1, KLRC1, KLRD1, KLRF1, KLRK1). Color scale reflects mean expression (log transformed TPM) across genes within each signature.

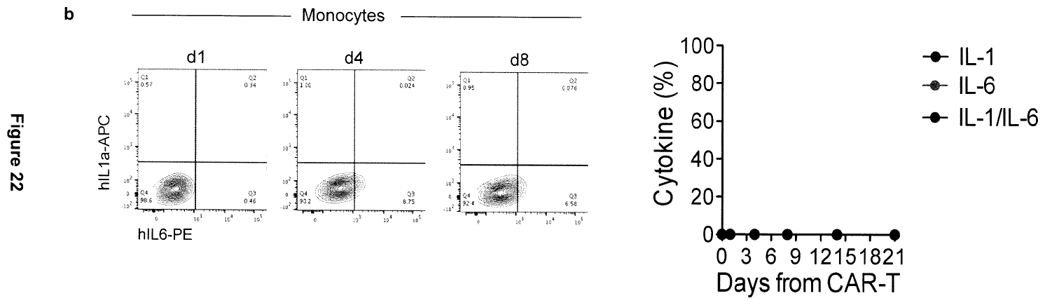

FIG. 24: Dynamic changes in the composition of human lympho-myeloid system in HuSGM3 mice during CRS. (a) Expression (scaled log transformed TPM values) of top 20 discriminative genes for each cluster is shown as heatmap. Selected representative genes for each cluster are shown on the right. Up to 200 single cells are shown for each cluster. (b) tSNE plot incorporating scRNA-Seq data of human CD45+ cells sorted from the spleen of leukemic HuSGM3 mice infused with CD19.28z CAR-T cells at day 2 and day 7 of CRS. Each dot is colored based on the respective experimental sample and replicate, as shown in the legend. Clusters, as defined in FIG. 6e, are indicated by circled numbers.

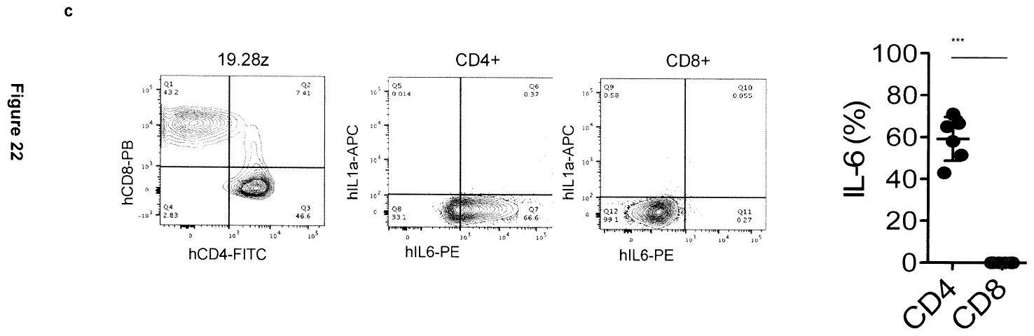

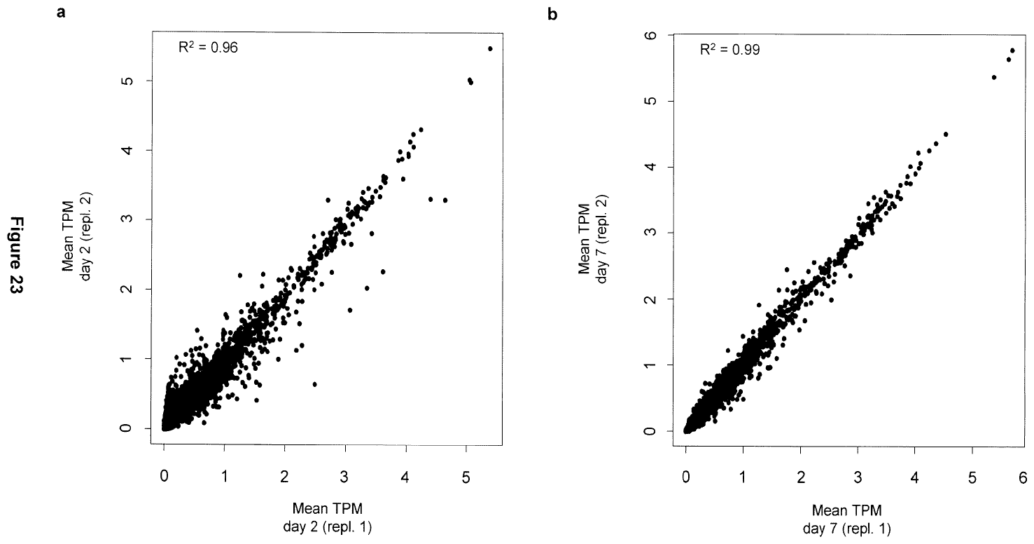

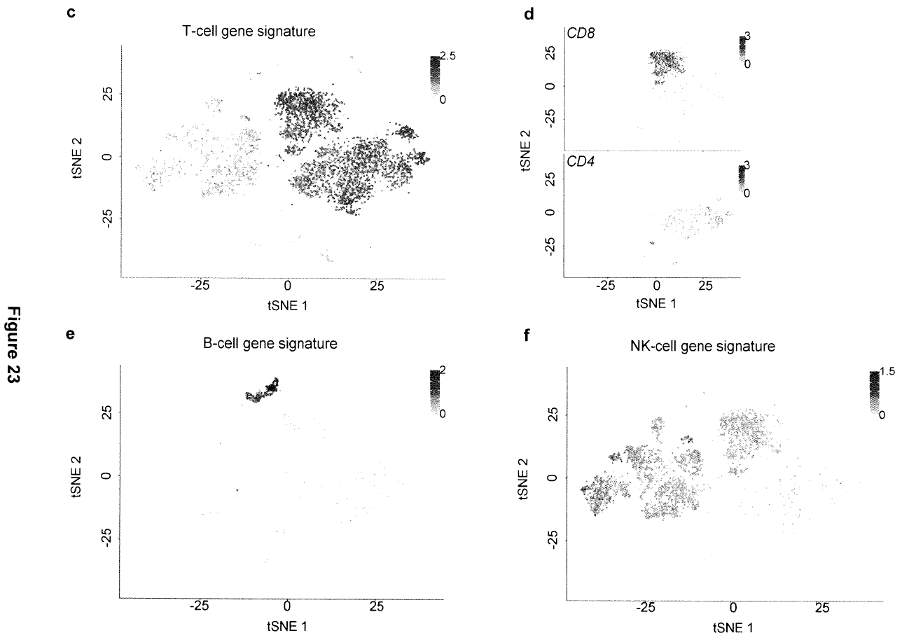

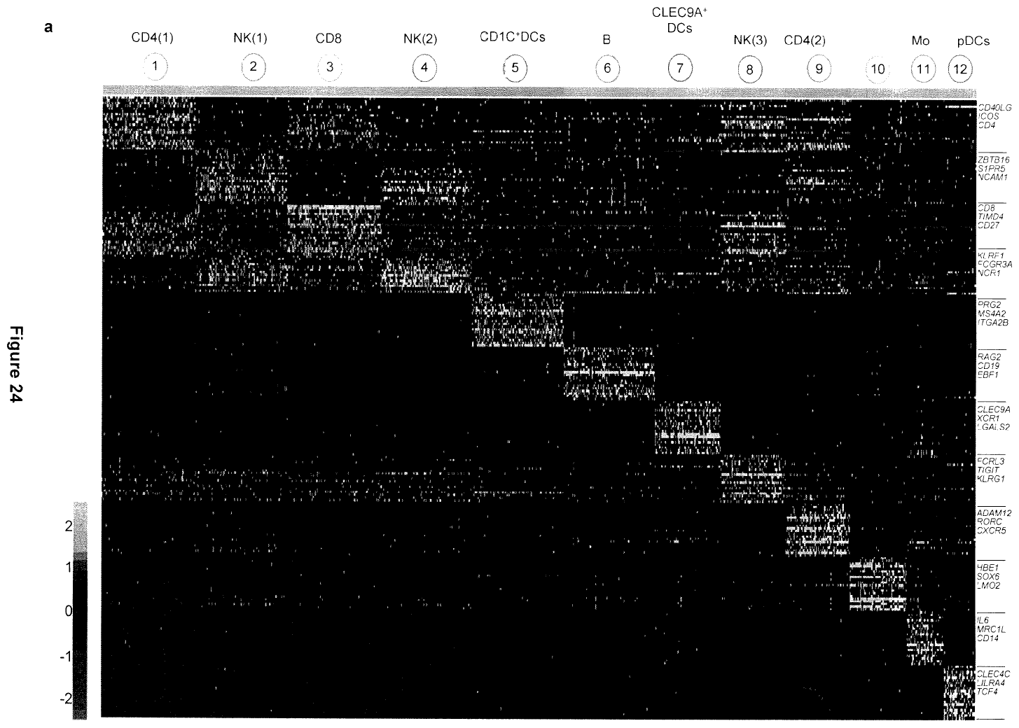

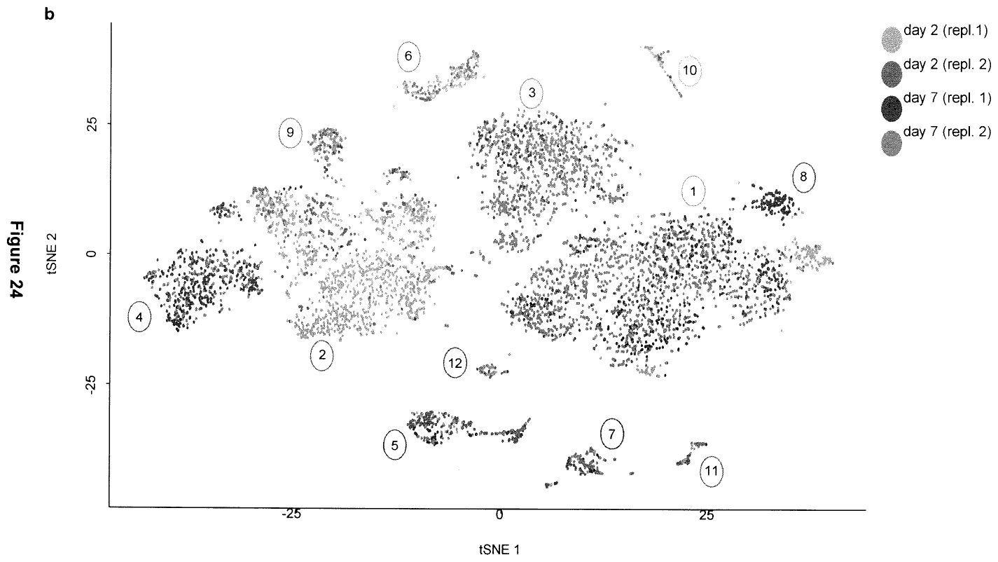

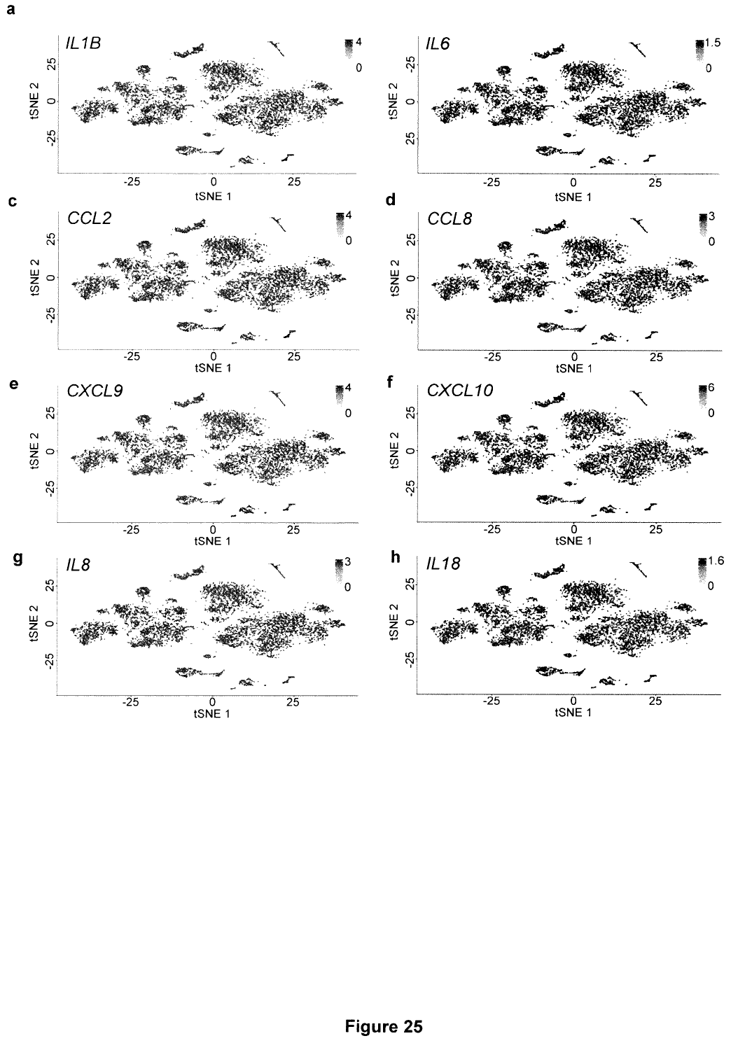

FIG. 25. Myeloid-specific expression of genes encoding for inflammatory cytokines and chemokine in leukemic HuSGM3 mice during CRS. (a-h) tSNE plots showing single-cell expression levels of the indicated genes. Color scale reflects gene expression in log(TPM+1).

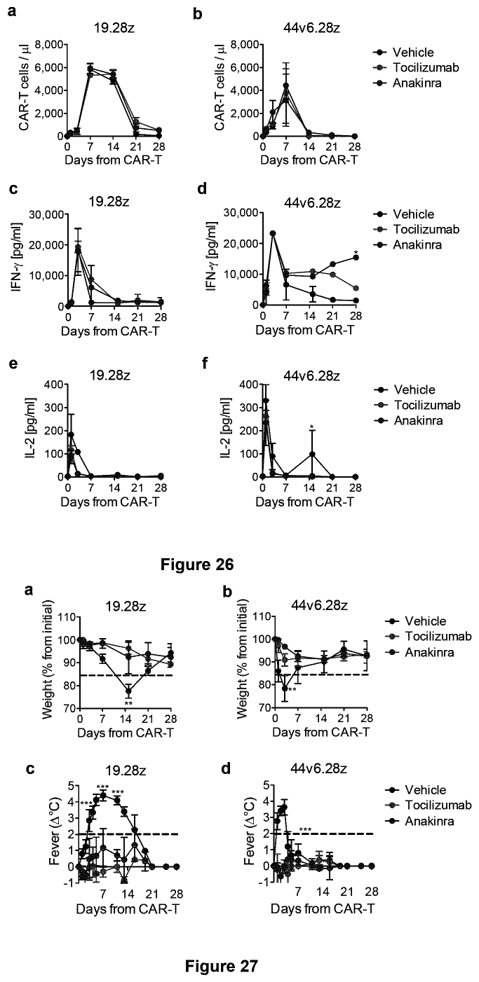

FIG. 26: CAR-T cell expansion after tocilizumab/anakinra prophylaxis. (a-b) Adult SGM3 mice were co-infused i.v. with 10⁵ CB HSCs (n=3 donors, HuSGM3) and 5×10⁶CD19+CD44v6+ ALL-CM leukemic cells, and, after 7 weeks with HuSGM3 T cells (same HSC donors) that had been ex vivo engineered with a CD44v6.28z CAR (44v6.28z, n=50 mice from three independent experiments) or a CD19.28z CAR (19.28z, n=50 mice). Just before CAR-T cells, mice received vehicle (n=14 per group), tocilizumab (n=18 per group) or anakinra (n=18 per group). HuSGM3 CAR-T cell expansion kinetics are shown as mean±SD. (c-f) Mean human IFN-g and IL-2, concentrations±SD over days from CAR-T cells are shown. Results from a two-way ANOVA test with Bonferroni correction are indicated when statistically significant. (*, P<0.05).

FIG. 27: CRS prevention by tocilizumab/anakinra. (a-b) Adult SGM3 mice were co-infused i.v. with 10⁵ CB HSCs (n=3 donors, HuSGM3) and 5×10⁶ CD19⁺CD44v6⁺ ALL-CM leukemic cells, and, after 7 weeks with HuSGM3 CD44v6.28z (n=50 mice from 3 independent experiments) or CD19.28z (n=50 mice) CAR-T cells (same HSC donors). Just before CAR-T cells, mice received vehicle (n=14 per group), tocilizumab (n=18 per group) or anakinra (n=18 per group). Mean percentages of body-weight variations±SD over days from CAR-T cells. Dashed line indicate the threshold for severe weight loss (>15%). (c-d) Mean body-temperature variations±SD over days from CAR-T cells. Dashed line indicate the threshold for high fever (DT>2° C.). Results from a two-way ANOVA test with Bonferroni correction are indicated when statistically significant. (**, P<0.01; ***, P<0.001).

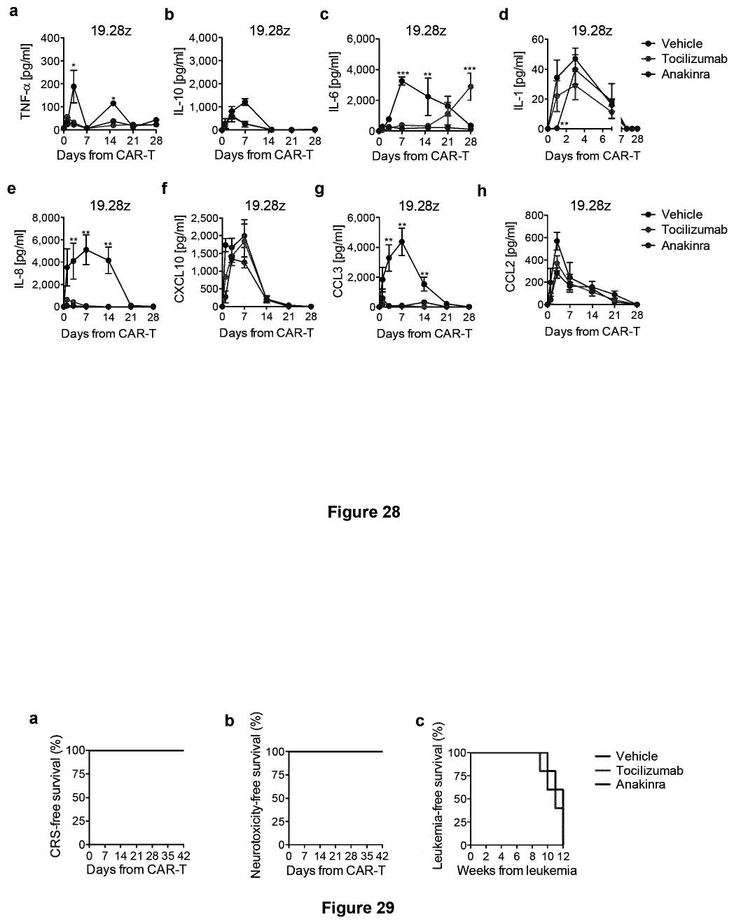

FIG. 28: Cytokine/chemokine kinetics after tocilizumab/anakinra prophylaxis. (a-h) Mean human TNF-a, IL-10, IL-6, IL-1, IL-8, CXCL10, CCL3 and CCL2 serum concentrations±SD over days from CD19.28z CAR-T cells are shown. Results from a two-way ANOVA test with Bonferroni correction are indicated when statistically significant. (*, P<0.05; **P<0.01; ***,P<0.001).

FIG. 29: Lack of neurotoxicity in leukemic HuSGM3 mice infused with irrelevant EGFR.28z CAR T cells and receiving tocilizumab/anakinra prophylaxis. (a) Adult SGM3 mice were co-infused i.v. with 10⁵ CB HSCs (n=2 donors, HuSGM3) and 5×10⁶ CD19⁺CD44v6⁺ ALL-CM leukemic cells, and, after 7 weeks with HuSGM3 EGFR.28z (n=18 mice from 2 independent experiments) CAR-T cells (same HSC donors). Just before CAR-T cells, mice received vehicle (n=6 per group), tocilizumab (n=6 per group) or anakinra (n=6 per group). CRS-free survival, (b) neurotoxicity-free survival and (c) leukemia-free survival over days from CAR-T cells are shown.

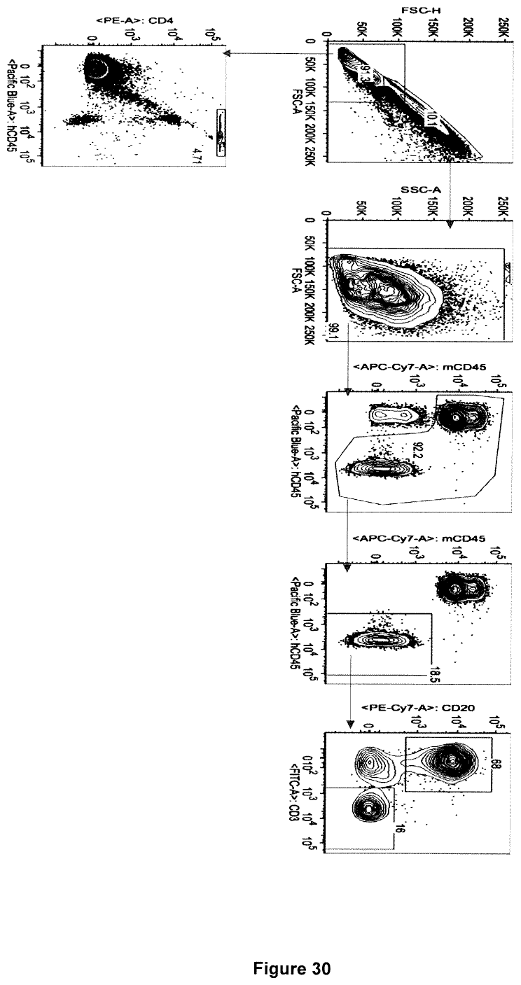

FIG. 30: Gating strategy exemplification. Mouse peripheral blood was stained with antibodies, lysed with ACK and acquired through a FACS Canto II apparatus. Serial gating is shown for cells (upper row) and counting fluorospheres (lower panel).

### DETAILED DESCRIPTION OF THE INVENTION

Methods

Generation of CAR constructs. CAR constructs were generated by gene synthesis of scFVs specific for CD44v6 (BIWA-8) or CD19 (FMC63), fused to a nerve growth factor receptor-derived spacer (NGFR), a transmembrane domain, a costimulatory endodoman from either CD28 (28z) as described in WO 2016/042461 (incorporated by reference), 4-1BB (BBz) or OX40 (zOX), and the CD3 zeta chain. In case of CD28 endodomains, the transmembrane domain was also derived from CD28. In all other cases, it was derived from CD4. All constructs were expressed in SFG RV vectors. RV supernatants were produced in 293T cells.

Cells and culture conditions. PB mononuclear cells were derived from healthy blood donors. CB mononuclear cells were supplied by commercial vendors (Lonza). CD34⁺ HSCs were isolated with immunomagnetic beads (Miltenyi). All procedures were approved by the Institutional Review Board (IRB number: TIGET_01) of San Raffaele University Hospital and Scientific Institute and human material obtained after written informed consent. Leukemic cell lines (THP-1, BV173) were purchased from ATCC.

THP-1 leukemia progression was followed in vivo by ultrasound imaging of the liver, where this cell line spreads forming myeloid sarcomas. ALL-CM leukemic cells were derived from patient with chronic myeloid leukemia in lymphoid blast crisis. CD44v6 was expressed in ALL-CM leukemic cells by lentiviral (LV) transduction. T cells were activated with CD3/CD28-beads (InVitrogen) at 3:1 ratio and 5 ng/ml IL-7/IL-15, and RV transduced by spinoculation at day 2 and 3. At day 6, beads were removed and T cells cultured in X-VIVO 10 (BioWhittaker) plus 10% FBS (Lonza). Transduction efficiency was determined by staining with an anti-NGFR mAb reactive with the CAR spacer. T cell expansion is expressed as fold increase: T cell numbers at day 14/T cell numbers at day 0. DCs were generated by culturing NSG mouse bone marrow, PB or CB adherent fractions with GM-CSF/IL-4 for 6 days, followed by LPS maturation overnight.

Flow cytometry. Mouse monoclonal Abs specific for human CD3 (BV510-conjugated, clone OKT3, Biolegend, lot nr. B226707; APC-Cy7-conjugated, clone SK7, Biolegend, lot nr. B225054), CD4 (PerCP-conjugated, clone SK3, BD Biosciences, lot nr. 23-5127-01), CD8 (APC-Cy7-conjugated, clone SK1, Biolegend, lot nr. B209571), CD14 (PerCP-conjugated, clone MϕP9, BD Biosciences, lot nr. 23-5143-01), CD15 (BV510-conjugated, clone W6D3, Biolegend, lot nr. B201379), CD19 (PE-conjugated, clone HIB19, Biolegend, lot nr. B188908), CD33 (PE-conjugated, clone WM53, Biolegend, lot nr. B195145), CD44v6 (PE-conjugated, clone 2F10, R&D, lot nr. YAV0616061; APC-conjugated, clone 2F10, R&D, lot nr. YAW0515041), CD45 (APC-Cy7-conjugated, clone HI30, Biolegend, lot nr. B214034; PE-Cy7-conjugated, clone HI30, Biolegend, lot nr. B210429), CD45RA (FITC-conjugated, clone HI100, Biolegend, lot nr. B202186), CD62L (APC-conjugated, clone DREG-56, Biolegend, lot nr. B230061), CD95 (PE-conjugated, clone DX2, Biolegend, lot nr. B2013943), NGFR (PE-conjugated, clone C40-1457, BD Biosciences, lot nr. 7068641), IL-6 (PE-conjugated, Miltenyi Biotec, lot nr. 5171106502), IL-1 (APC-conjugated, Miltenyi Biotec, lot nr. 5171106567), and a rat mAb specific for mouse CD45 (Ly5.1; PerCP-conjugated, clone 30-F11, Biolegend, lot nr. B214531) were purchased from commercial vendors. Samples were run through a FACS Canto II flow cytometer (BD Biosciences) and data were analyzed with the FlowJo software (LLC). An example of gating strategy is shown in FIG. 30.

In vitro functional assays. CAR-T cells were cultured with target cells at different E:T ratios. After 24 hrs, co-culture supernatants were collected and subsequently analyzed with the LEGENDplex bead-based cytokine immunoassay (Biolegend). After four days, surviving cells were counted and analyzed by FACS. T cells transduced with an irrelevant CAR (GD2-specific or EGFR-specific) were always used as control. Elimination index was calculated as follows: 1−(number of residual target cells in presence of experimental CAR-T cells)/(number of residual target cells in presence of CTRL CAR-T cells). In CFSE-diluting assays, T cells were loaded with CFSE and stimulated with irradiated (10′000 cGy) splenocytes from NSG or CD57/Bl6 mice, or with irradiated human allogeneic PB mononuclear cells at 1:5 E:S ratio. After 6 days, T cell proliferation was measured by FACS.

Mouse experiments. All mouse experiments were approved by the Institutional Animal Care and Use Committee (IACUC) of San Raffaele University Hospital and Scientific Institute and by the Italian Governmental Health Institute (Rome, IT). Eight-to-ten weeks old female or male NSG (NOD.Cg-Prkdc^{scid} II2rgt^{m1Wjl}) or SGM3 mice (NSG Tg^{CMV-IL3,CSF2,KITLG}1Eav/MloySzJ; Jackson Laboratories) were screened by PCR (according to JAX instructions and primers, stock number 013062) and ELISA (R&D systems; Catalog numbers DCK00, DGM00 and D3000 for SCF, GM-CSF and IL-3, respectively), for transgene expression and human cytokine expression. Newborn (0-2 days from birth) female of male NSG or SGM3 mice were sub-lethally irradiated (150 cGy from a linear accelerator) and injected intra-liver with 1×10⁵ human CB CD34⁺ cells. Adult mice were sub-lethally irradiated (200 cGy) and immediately i.v. infused with 1×10⁵ human CB CD34⁺ cells. For assessing X-GVHD, mice were monitored daily for hunching, activity, fur texture, skin integrity and weight loss. For studying CAR-T cell toxicities, mice were followed daily for weight loss and body temperature by rectal thermometer, and weekly for mouse SAA, uric acid and human cytokine levels by LegendPLEX bead-based cytokine immunoassay (Biolegend). For evaluating antileukemia efficacy, mice were infused i.v. with THP-1 (1×10⁶) or ALL-CM (5×10⁶ or 10×10⁶ for mice in FIG. 2) leukemic cells and, after 5 or 7 weeks (low or high tumor burden, respectively) with 2×10⁶ CAR-T cells. Leukemic and CAR-T cell counts were monitored weekly in peripheral blood by FACS using Flow-Count Fluorospheres (BeckmanCoulter). Mice were euthanized when weight loss was >20% or when manifesting signs of inhumane suffering. For depleting phagocytes, mice were treated i.p. with liposomal clodronate ClodronateLiposomes.com) for three consecutive days prior to CAR-T cell infusion. Tocilizumab (10 mg/kg, Roactemra, Roche) or anakinra (10 mg/kg, Kineret, Amgen) were administered i.v. immediately before CAR-T cells. While tocilizumab was given only once, anakinra administration was repeated daily for 7 days because of the different pharmacokinetics.

Single-Cell RNA Sequencing

Droplet-based digital 3′ end scRNA-Seq was performed on a Chromium Single-Cell Controller (10X Genomics, Pleasanton, Calif.) using the Chromium Single Cell 3′ Reagent Kit v2 according to the manufacturer's instructions. Briefly, suspended single cells were partitioned in Gel Beads in Emulsion (GEMs) and lysed, followed by RNA barcoding, reverse transcription and PCR amplification (12-14 cycles). Sequencing-ready scRNA-Seq were prepared according to the manufacturer's instructions, checked and quantified on 2100 Bioanalyzer (Agilent Genomics, Santa Clara, Calif.) and Qubit 3.0 (Invitrogen, Carlsbad, Calif.) instruments. Sequenced was performed on a NextSeq 500 machine (Illumina, San Diego, Calif.) using the NextSeq 500/550 High Output v2 kit (75 cycles).

Computational Methods

Raw reads were processed and aligned to the ENSEMBL hg19 transcriptome using CellRanger version 1.3 (https://support.10xgenomics.com/single-cell-gene-expression/software/pipelines/latest/what-is-cell-ranger) with default parameters. Only confidently mapped reads, non-PCR duplicates, with valid barcodes and UMIs (Unique Molecular Identifiers) were retained. The inventors filtered out low quality cells. A minimum of 500 unique genes detected for cell was required, additionally cells with a ratio of mitochondrial versus endogenous gene expression exceeding 0.1 were discarded. Resulting 6511 cells were retained for further analysis. Gene expression values were quantified in log transformed transcript per million [log(TPM+1)]. Downstream analyses were performed using the R software package Seurat version 2.1 (https://github.com/satijalab/seurat/). Cell clustering and tSNE analysis were performed on 1175 most variable genes, selected with mean expression higher than 0.01 and log transformed variance to mean ratio higher than 0.5.

Histopathological analysis. After hematoxylin and eosin staining, mouse organs including thymus and brain were blindingly and independently analyzed by at least two experienced NSG mouse pathologists (S. F and C. P.). Immunohistochemistry for TdT, human CD3 or CD68 was performed according to standard procedures. Meningeal thickening was scored according to the following arbitrary criteria: 0, normal; 1, mild; 2, moderate; 3, severe.

Statistics. Statistical analysis was performed by either one- or two-way ANOVA, by Mantel-Cox (log-rank) test or by a two-tailed Mann-Whitney test (Prism Software 5.0, Graphpad). Differences with a P value<0.05 were considered statistically significant. Sample size was calculated by power analysis with 0.05 alpha error and 0.80 power. For experiments on antileukemia efficacy (assumptions: leukemia progression in 100% of control mice vs 50% in treated mice), power analysis returned a n=11 size per experimental group. For experiments on tocilizumab or anakinra effectiveness (assumptions: CRS mortality in 35% of control mice vs 0% in treated mice), power analysis returned a n=17 size per experimental group. Before any treatment, mice were blindly randomized and no sample or animal was excluded from analysis.

### EXAMPLES

T Cells from HuSGM3 Mice are Non-Xenoreactive and can be Redirected Against Leukemia by CAR Engineering

Aiming at the development of a xenograft mouse model for studying the specific contribution of myeloid cells to CAR-T cell toxicities, the inventors transplanted human cord blood (CB) hematopoietic stem cells (HSCs) by intra-liver injection into sub-lethally irradiated newborn NSG-SGM3 (HuSGM3) mice and initially profiled lympho-hematopoietic reconstitution. Compared with control HuNSG mice, HuSGM3 mice reconstituted human CD45⁺ hematopoiesis more rapidly (FIG. 8a), displaying lower counts of CD19⁺ B cells (FIG. 1a), but inversely higher counts of CD33⁺ myeloid cells (FIG. 8b), CD14⁺ monocytes (FIG. 1b), and CD15⁺ granulocytes (FIG. 8c). HSC humanization of newborn SGM3 mice also resulted in robust CD3⁺ T cell development (FIG. 1c), which contrariwise was negligible when mice were humanized in adulthood (FIG. 8d-f). The timing of HSC injection soon after birth was critical to successful human T lymphopoiesis, since a two-days delay almost completely dampened the effect (FIG. 9a). Circulating T cells in HuSGM3 mice displayed a physiological CD4/CD8 ratio (FIG. 1d) and over time appeared to differentiate from CD45RA⁺CD62L⁺ naïve (T_{Na}), to CD45RA⁻CD62L⁺ central memory (T_{CM}) to CD45RA⁻CD62L⁻ effector memory (T_{EM}) cells (FIG. 1e). Only a minority of CD45RA⁺CD62L⁺ T cells expressed the stem cell memory (T_{SCM}) marker CD95³⁹ (FIG. 9b). T cell development in HuSGM3 mice was associated with substantial thymus cellularity (at 12 weeks of age, mean 0.99×10⁶±0.59 SD), including single positive CD4/CD8 T cells (FIG. 9c), and an architecture characterized by distinct cortical and medullary areas (FIG. 1f), populated with human CD3⁺ T cells by immunohistochemistry (FIG. 1g). Spleen (mean 3.79×10⁶±1.50 SD; FIG. 9d) and bone marrow (mean 1.71×10⁶±1.17 SD; FIG. 9e) were also colonized by human T cells. Intrigued by the observation of sizeable T lymphopoiesis in HuSGM3 mice, the inventors next addressed the issue of their functionality. In vitro, HuSGM3 T cells were hypo-responsive to NSG mouse antigens (I-A⁹⁷), but vigorously proliferated in response to C57/Bl6 mouse antigens (I-A^{d}) and to human alloantigens (FIG. 1h). Moreover, once i.v. transferred into sub-lethally irradiated secondary NSG recipients, HuSGM3 T cells failed to induce X-GVHD (FIG. 1i) yet persisted at low levels up to 24 weeks (FIG. 1l). The functionality of secondarily transferred HuSGM3 T cells in vivo was confirmed by expansion in response to vaccination with human allogeneic, but not with autologous CB-derived or NSG mouse dendritic cells (DCs; FIG. 1m). To establish bio-equivalence with CAR-T cells from humans, HuSGM3 T cells were activated with CD3/CD28-beads and IL-7/IL-15 ex vivo, according to a protocol that preserves early-differentiated (T_{SCM}/T_{CM}) memory T cells⁴⁰⁻⁴² and subsequently engineered with anti-CD44v6 CARs of different designs (28z, BBz, zOX) by retroviral (RV) transduction. Transduction and expansion rates were slightly inferior to those of T cells from human peripheral blood (PB), but superimposable to those of CB T cells (FIG. 10a-b). After CAR engineering, CD4/CD8 ratios and memory differentiation phenotypes were conserved (FIG. 10c-d). HuSGM3 T cells engineered with CD44v6.28z or CD44v6.zOX CAR, but not with CD44v6.BBz CAR, specifically and effectively killed CD44v6⁺ THP-1 leukemic cells in vitro (FIG. 1n), produced IFN-γ and secondarily proliferated (FIG. 11a-b). CD44v6.BBz CAR-T cells from human PB were also weakly effective (FIG. 11c-e), indicating that suboptimal functionality was due to this particular design, rather than to T cell source. Accordingly, there were no differences between 28z and BBz designs in case of HuSGM3 T cells transduced with CD19 CARs (FIG. 11f-h). Once infused i.v. into mice previously engrafted with THP-1 leukemic cells, CD44v6.28z CAR-T cells from HuSGM3 mice were as potent as those from human PB in controlling leukemic outgrowth (FIG. 12a-d). Moreover, HuSGM3 CAR-T cells were progressively enriched for transgene expression, confirming lack of xenoreactivity (FIG. 12e).

Leukemia Clearance by CAR-T Cells in HuSGM3 Mice Associates with CRS

To evaluate the antileukemia efficacy of CAR-T cells specific for CD19 and CD44v6 in vivo using the same xeno-engrafting tumor cells, the inventors transduced patient-derived CD19⁺ ALL-CM leukemic cells with different CD44 isoforms containing or not the variant 6 (FIG. 13a-b). After initial remission, CD44v6⁺CD19⁺ ALL-CM leukemia-bearing mice infused with CD44v6.BBz CAR-T cells eventually relapsed (FIG. 13c), while those receiving either CD44v6.28z, CD19.BBz or CD19.CD28z CAR-T cells benefited from durable antileukemic effects (FIG. 13d). For sake of comparability, all subsequent experiments were therefore performed with either CD19.28z or CD44v6.28z CAR-T cells.

The inventors next exploited HuSGM3 CAR-T cells for mimicking early toxicities associated with antileukemic effects in the absence of confounding xenoreactivity (FIG. 2a). Adult SGM3 mice were engrafted with ALL-CM leukemic cells and later infused with either CD19.28z or CD44v6.28z CAR-T cells after 5 weeks (low leukemia burden, circulating leukemic cells: mean 20.2±13.1 SD; FIG. 2b) or after 7 weeks (high leukemia burden, circulating leukemic cells: mean 2811.0±390.2 SD; FIG. 2c). In either setting, CD44v6.28z or CD19.28z CAR-T cells mediated rapid and long-lasting leukemia clearance in peripheral blood. However, only in case of high leukemia burden, CAR-T cells robustly expanded in vivo (FIG. 14a-b) and SGM3 mice developed a transient syndrome (median duration: 7 days, range 3-10), characterized by moderate weight loss (<15% from initial; FIG. 2d-e) and mild fever (ΔT<2° C. from basal; FIG. 2f). These signs were paralleled by increased systemic levels of human IFN-γ and IL-2, but not of TNF-α, IL-10 and IL-6 (FIG. 2g). The levels of serum amyloid A (SAA), murine homolog to the human CRS biomarker C-reactive protein¹⁷, whose production is under IL-6 control, were also unchanged (FIG. 2h). These data, along with a transient rise in uric acid (FIG. 2i), were therefore more indicative of tumor lysis syndrome, rather than of CRS. Long-term antileukemia efficacy by HuSGM3 CAR-T cells was confirmed by high rates of deep remission (bone marrow leukemic cells<5%; FIG. 14c) at 24 weeks from infusion, without differences between mice receiving CD19.28z (7/11 mice) or CD44v6.28z CAR-T cells (5/11). The 5% cut-off for deep remission was chosen based on subsequent experiments demonstrating lack of engraftment in tertiary recipients in case of residual bone marrow leukemic cells below this threshold (FIG. 14d-e).

Endogenous myeloid cells from immunocompromised mice derived from the NOD background are known to be functionally defective⁴³⁻⁴⁵. Aiming at modeling human CRS, the inventors therefore infused non-xenoreactive HuSGM3 CAR-T cells into secondary recipients previously humanized with HSCs, as a way for simultaneously providing functional myeloid cells (FIG. 3a) and antigenic CD19⁺ B cells or CD44v6⁺ monocytes (FIG. 15a). As expected, CD19.28z and CD44v6.28z CAR-T cells expanded in vivo, although with different kinetics (FIG. 15b), and induced long-lasting B cell (FIG. 3b) and monocyte (FIG. 3c) aplasia, respectively. Moreover, despite a significant difference in circulating antigenic cells before infusion (CD19⁺ B cells per μl: mean 447.5±27.5 SD vs CD44v6⁺ monocytes per μl: mean 44.1±3.1 SD, P<0.05 by Mann-Whitney test), CD19.28z and CD44v6.28z CAR-T cells equivalently caused a violent systemic inflammatory syndrome, highly reminiscent of human CRS and characterized by severe weight loss (>15% from initial; FIG. 3d), increased systemic human IL-6 levels (FIG. 3e) and high fever (ΔT>2° C. from basal; FIG. 3f). Elevations of systemic human TNF-α and IL-10 (FIG. 15c-d), as well as of IL-6-induced mouse SAA (FIG. 3g), closely mirrored the kinetics of the syndrome. All these signs were negative in control SGM3 mice not previously humanized with HSCs. Interestingly, it was evident that CRS by CD44v6.28z CAR-T cells was somewhat anticipated and shorter than that by CD19.28z CAR-T cells, although resulting in comparable mortality (25% vs 33.3%). At histopathology, mice dying from CRS had human CAR-T cell infiltration in the liver, often accompanied by a human histiocytic component (not shown).

Monocytes are Major Sources of IL-1 and IL-6 Induced by CAR-T Cells in HuSGM3 Mice

The inventors next examined whether leukemia presence in HuSGM3 mice, and especially its burden, could be a determinant of CRS severity by CAR-T cells, as observed in humans¹⁷. To this aim, HuSGM3 mice were co-engrafted with ALL-CM leukemic cells and later infused with non-xenoreactive HuSGM3 CAR-T cells after verifying the establishment of different leukemia burdens. CRS by either CD19.28z or CD44v6.28z CAR-T cells was more severe in case of higher leukemia burden, as revealed by more profound weight loss (FIG. 4a-b), superior systemic levels of human IL-6 (FIG. 4c-d) and higher fever (FIG. 4e-f). Consequently, CRS mortality was also significantly different (FIG. 4g-h), correlating with in vivo kinetics of CAR-T cells (FIG. 16a-b) and with systemic human IFN-γ elevations (FIG. 16c-d). During CRS, the majority of mouse cytokines and chemokines were undetectable (FIG. 16e), suggesting a minor contribution, if any. Leukemic HuSGM3 mice infused with irrelevant EGFR.28z CAR-T cells as control did not develop CRS (FIG. 17a-e), but conversely died from leukemia within 8 weeks (FIG. 17f).

A highly relevant question to the CAR-T cell field is whether the type of costimulatory endodomain influences CRS liability. To answer this question, the inventors compared CRS incidence and severity by either 28z or BBz CAR-T cells specific for CD19 or CD44v6 in HuSGM3 secondary recipients with high leukemia burden. Despite differences in kinetics (FIG. 4i), CD44v6.BBz CAR-T cells unexpectedly caused significantly more severe CRS than CD44v6.28z CAR-T cells, resulting in 100% mortality (FIG. 4l-m). Disproportionate CRS mortality by CD44v6.BBz CAR-T cells was associated with inferior antileukemic effects, despite greater T cell activation in vivo (FIG. 18a-b), and a paradoxical surge in human monocyte counts (FIG. 18c). Such an effect was mirrored by increased systemic levels of human inflammatory cytokines (FIG. 18d-g) and, among monocyte-derived chemokines, of IL-8 and CCL3/MIP-1α (FIG. 18h-m). In line with results in humans, CD19.BBz CAR-T cells mediated similar antileukemic effects compared to CD19.28z CAR-T cells, without inducing excessive mortality (FIG. 4n-p).

While exploring the variables influencing CRS, the inventors noticed that, due to different timing from HSC humanization (7 vs 5 weeks) at the time of CAR-T cell infusion, SGM3 mice with higher leukemia burden concomitantly displayed superior monocyte counts (mean 57.9±18.3 SD per μl vs 26.8±9.8 SD per μl, P<0.0001 by Mann-Whitney test). To weigh monocyte contribution to CRS, the inventors took advantage of the observation that their reconstitution in HSC-humanized mice is strain and sex-dependent (FIG. 5a), whereas leukemia engraftment (FIG. 5b) and CAR-T cell kinetics (FIG. 19a) are not. CRS mortality by CD19.28z CAR-T cells proved higher in female HuSGM3 than in female HuNSG mice (FIG. 5c), correlating with superior monocyte counts. More directly, depleting monocytes before CD19.28z CAR-T cell infusion by liposomal clodronate administration (FIG. 19b-d) had no direct effect on B cell or leukemic cell counts and completely abated CRS incidence and mortality (FIG. 5d-e and FIG. 19e). At a closer look, it was however evident that monocyte depletion had a negative impact on in vivo CAR-T cell expansion (FIG. 19f) and on the kinetics of leukemia clearance (FIG. 5f). Similar results were observed with CD44v6.28z CAR-T cells (FIG. 5g-i). The adjuvant role of monocytes on overall antileukemia efficacy by CD19.28z CAR-T cells was confirmed in vitro in three-party co-culture experiments (FIG. 19g).

To demonstrate that monocytes were primarily responsible for CRS and contributed to the antileukemic effects by CAR-T cells, the inventors used CD44v6.28z CAR-T cells as a way to ablate monocytes long term in HuSGM3 mice, and subsequently challenged them with leukemia. In agreement with the inventors' hypothesis, mice rendered monocyte aplastic by prophylactic CD44v6.28z CAR-T cells, but not mice infused with CD19.28z CAR-T cells as control, were protected from CRS (FIG. 5l-m and FIG. 19h). In the absence of monocytes, decreased secondary in vivo expansion of CD44v6.28z compared to CD19.28z CAR-T cells (FIG. 19i) was however awkwardly associated with lower rates of deep remission at sacrifice (FIG. 5n).

Although IL-6 is recognized to be pivotal for CRS pathogenesis¹⁸, it is at present unknown whether CAR-T cells themselves might be major sources of this cytokine during the syndrome. To tackle this issue, the inventors set up an in vitro cytokine release assay by co-culturing CD19.28z or control EGFR.28z CAR-T cells with leukemic cells with or without THP-1 monocytic cells. In this assay, while GM-CSF and TNF-α (FIG. 20a-b) were released upon specific tumor recognition by CD19.28z CAR T cells alone, the production of IL-1, IL-6, IL-8, CCL3/MIP-1α, required THP-1 cells (FIG. 20c-f). Interestingly, a time course analysis revealed that IL-1 preceded IL-6 release by approximately 24 hrs (FIG. 20g). Intracellular staining results confirmed the kinetics of IL-1/IL-6 production both in vitro, in three-party co-cultures with primary autologous monocytes (FIG. 21a-d), and in vivo in leukemic HuSGM3 mice infused with CD19.28z (FIG. 6a-c), but not with control EGFR.28z CAR-T cells (FIG. 22a-b). In vivo, transient IL-6 production was also detected in CD19.28z CAR-T cells, limitedly to the CD4 subset (FIG. 22c).

To define the cellular determinants of CRS in a broader manner, the inventors performed single-cell RNA-Sequencing (scRNA-Seq) on whole human CD45⁺ leukocytes isolated from leukemic HuSGM3 mice infused with CD19.28z CAR-T cells, two days after CRS onset and 5 days later. Using a microfluidics-based approach⁴⁶, the inventors generated scRNA-Seq libraries from 6,511 cells and sequenced them at a median depth of 56,164 reads per cell. The average number of detected genes per cell was 1,980, with a very high correlation between replicates (R²>0.9; FIG. 23a-b). Clustering analysis, performed using a graph-based approach^{47,48} identified 12 clusters (cl.) encompassing the major human lymphoid and myeloid cell populations (FIG. 6d). Using unbiased gene signature analysis (FIG. 23c-f), the inventors defined populations of CD4 T cells (cl. 1 and 8), CD8 T cells (cl. 3), NK-like cells (cl. 2 4 and 9), B cells (cl. 6), as well as a poorly defined population (cl. 10). The inventors also identified monocytes (cl. 11), two sub-populations of conventional DCs, respectively expressing FCERIA and CLEC9A (cl. 5 and 7, respectively), and plasmacytoid DCs⁴⁸ (cl. 12; FIG. 6d, FIG. 24a) Cell populations showed different dynamics during CRS, with monocytes and DCs being detected at both time points (FIG. 24b). As expected, cl. 6, comprising both B cells and leukemic cells, was present at the earlier time point, but disappeared later on, mirroring on-target clearance of CD19⁺ cells. Contrariwise, cl. 1, 8 and 3 were selectively enriched, reflecting CAR-T cell expansion. At the single-cell level, monocytes specifically expressed high levels of IL1B and IL6, as well as of IL8, CCL2, CCL8 and CXCL10 (encoding for IL-8, MCP-1, MCP-2 and IP-10, respectively; FIG. 6e). This comprehensive analysis revealed that, at least to some extent, also DCs expressed inflammatory genes, including CXCL9 and IL18 at high levels (FIG. 6e, FIG. 25a-f).

Anakinra, but not Tocilizumab, Protects HuSGM3 Mice from Lethal Neurotoxicity by CAR-T Cells

In humans, tocilizumab is often used, either alone or in combination with steroids, to manage CAR-T cell toxicities, ameliorating fever and hypotension typical of severe CRS, but apparently failing to revert severe neurotoxicity^{10-12,17}. Despite anecdotal reports, ample data on CRS responsiveness to anakinra, an IL-1 receptor antagonist, are lacking. Motivated by the in vitro observation of early IL-1 induction in monocytes by CAR-T cells, the inventors used the inventors' xenograft mouse model of human CRS to verify whether anakinra might have some advantages over tocilizumab. At the time of CAR-T cell infusion, cohorts of leukemic HuSGM3 mice were administered either tocilizumab, anakinra, or vehicle as control. Either drug did not substantially interfere with in vivo CAR-T cell expansion (FIG. 26a-b) or in vivo IFN-γ and IL-2 production (FIG. 26c-f), and was effective at preventing CRS by both CD19.28z and CD44v6.28z CAR-T cells (FIG. 7a-b and FIG. 27a-d). CRS prevention by tocilizumab was associated with early normalization and a later increase in systemic human IL-6 levels (FIG. 28a-c). Initial normalization of systemic human IL-1 levels by anakinra was not followed by a similar increase (FIG. 28d), possibly due to a different pharmacology in mice compared to humans. Systemic human IL-8 and CCL3/MIP-1α levels were protractedly abated by either drug (FIG. 28e-h). Importantly, leukemia clearance by CAR-T cells in HuSGM3 mice receiving either tocilizumab or anakinra was similar to that in control mice (FIG. 7c-d).

By prolonging follow-up for detecting potential leukemia relapses, after a median of 30 days (range 27-33), in HuSGM3 mice prophylactically receiving either vehicle or tocilizumab, but not in those receiving anakinra, the inventors unexpectedly documented the occurrence of a sudden (24 hrs duration) and highly lethal neurological syndrome (FIG. 7e-f), characterized by generalized paralysis and, in some cases, by spontaneous convulsions. This form of delayed neurotoxicity was common to both CD19.28z and CD44v6.28z CAR-T cells and emerged only in mice with previous CRS (P<0.01 by Fisher's exact test, not shown). Post-mortem analysis did not reveal any sign of X-GVHD in target organs (skin and liver, not shown), but conversely showed multi-focal brain meningeal thickening, without leukemic cell infiltration in the CNS (FIG. 7g). Meningeal thickening, accompanied by human macrophage infiltration in subarachnoid space, as ascertained by scattered positivity for CD68 by immunohistochemistry (FIG. 7h), was effectively prevented by anakinra, but not tocilizumab (FIG. 7i). As a result, only anakinra prophylaxis had a statistically significant effect on overall survival (FIG. 7l-m). HuSGM3 mice infused with control EGFR.28z CAR-T cells did not develop either CRS or neurotoxicity but died from leukemia within 12 weeks (FIG. 29a-c).

The inventors finally investigated whether administering tocilizumab or anakinra to leukemic HuSGM3 mice after, rather than before, the onset of CRS by CD19.28z CAR-T cells (FIG. 7n) could revert the syndrome. Also, in this therapeutic setting, either drug was confirmed to be effective at decreasing CRS mortality, although with borderline statistical significance for anakinra (FIG. 7o). Nonetheless, anakinra treatment was uniquely associated with rescue from lethal neurotoxicity (FIG. 7p). Leukemia clearance by CAR-T cells was unaffected by either treatment (FIG. 7q).

The cellular and molecular players involved in life-threatening toxicity by cell therapy, in particular CAR-T cells in humans remain poorly understood. For gauging into its pathogenesis, the inventors used T cells derived from HSC-humanized SGM3 mice, a strain known to better support human lympho-hematopoiesis compared to NSG mice, including the development of myeloid and T cells³². Successful thymic education of human T cells in SGM3 mice was implied by their robust xenotolerance, a prerequisite for unbiased studies on CRS and neurotoxicity in secondary recipients. Although the reasons for efficient human T cell development in SGM3 mice are at present unknown, it is reasonable that transgenic expression of c-kit ligand/stem-cell factor might be key, as this cytokine is known to sustain thymopoiesis in immunocompromised mice transplanted with human HSCs⁴⁹. Either transgenic expression of HLA molecules^{50,51} or co-transplantation of human thymic tissue⁵² has been successfully used for boosting thymopoiesis in xenograft models and, in the future, would be worth combining with transgenic SCF in order to further improve human T cell development in NSG mice. In the present invention, by transferring non-xenoreactive CAR-T cells in leukemic HSC-transplanted SGM3 mice, the inventors demonstrated at the single-cell level, by both scRNAseq and flow cytometry, that human circulating monocytes are primarily responsible for the systemic release of IL-6, which ultimately cause the clinical manifestations of CRS. In this human xenograft mouse model of CRS, mouse cytokines, and IL-6 in particular, did not appear to play a significant role, likely due to cytokine dysregulation inherited from the NOD background⁵³.

In humans, circulating monocytes can be divided in different subpopulations according to their ability to phagocytose (classical monocytes, CD14⁺CD16⁻), produce proinflammatory cytokines (intermediate monocytes, CD14⁺CD16⁺) or patrol endothelial integrity (non-classical monocytes, CD14^{lo}CD16⁻)⁵⁴. In the inventors' model, besides proinflammatory monocytes, DCs were also involved in cytokine production, as revealed by unbiased and comprehensive in vivo scRNA-Seq analysis, underlying unexpected complexities, but also suggesting new cellular and molecular targets for therapeutic intervention. Since in the present invention the inventors have used leukemic cells that, besides obvious bone marrow homing, essentially accumulate in the circulation⁴¹, it is reasonable that intravascular leukemia recognition by CAR-T cells might have been crucial for licensing human circulating myeloid cells to produce inflammatory cytokines. Although the inventors cannot exclude that in tumors in which malignant cells do not routinely circulate in blood, e.g. lymphoma, the role of proinflammatory monocytes could be less prominent, the inventors' findings might explain the apparently higher incidence of severe CRS by CD19 CAR-T cells reported in human ALL⁹⁻¹², as compared to NHL¹³⁻¹⁶.

While human T cells are known to produce IL-6, the major source of this cytokine in vivo are monocytes/macrophages⁵⁵. Confirming recent findings⁵⁶, the inventors found that upon tumor recognition in vitro, CAR-T cells produce negligible levels of IL-6, whose release conversely requires by-stander monocytes. Quite unexpectedly, however, the inventors also observed that monocytes are licensed by CAR-T cells to produce IL-1, with a kinetics that precedes IL-6 by many hours. Since IL-1 is capable of inducing the secretion of IL-6, as well as of its soluble IL-6R (sIL-6R)⁵⁵, it is tempting to speculate that CRS by CAR-T cells in HSC-humanized SGM3 mice, and in humans, might be primarily initiated by IL-1 release from circulating monocytes. The inventors' in vivo scRNA-Seq and flow-cytometry data are in line with this hypothesis. Accordingly, in the inventors' human xenograft model, antagonizing IL-1 by in vivo administration of a IL-1 antagonist, such as anakinra, was equally effective at protecting mice from CRS mortality as blocking IL-6 trans-signaling, i.e. signaling derived from IL-6 coupling to sIL-6R, through tocilizumab. Most importantly, administration of either drug did not result in decreased antileukemic effects, even if given preemptively, suggesting that pharmacological CRS prophylaxis could be routinely adopted, without jeopardizing antileukemia efficacy.

Neurotoxicity by CD19 CAR-T cells, whose acknowledgement as a separate clinical entity was initially challenged by neurological manifestations of CRS¹⁹, is becoming an emerging issue. The recent halt to some ongoing CD19 CAR-T cell trials for lethal neurotoxicity has emphasized the need of a better understanding of this severe adverse event, especially in light of further clinical development and ongoing commercialization. The inventors were surprised to find that, besides CRS, the inventors' human xenograft mouse model of CAR-T cell therapy also recapitulated neurotoxicity, which was delayed, abrupt and highly lethal, mimicking a pattern often observed in humans. Another similarity with humans was that neurotoxicity by CAR-T cells in mice was seemingly unrelated to leukemia recognition in the CNS, as indicated by no evidence of leukemic localization at brain histopathology. Instead, mice dying from neurotoxicity displayed signs of meningeal inflammation, suggesting blood-brain barrier leakage to peripherally produced cytokines, as recently described in humans⁵⁷. As clinical data are accumulating, it is emerging that neurotoxicity by CAR-T cells may be more diversified than initially assumed, both in timing and relationship with CRS, possibly reflecting a combination of different mechanisms. Far from asserting that the specific type of neurotoxicity observed in the inventors' model may fit all varieties, the inventors' findings appear particularly relevant from a clinical standpoint. By analogy with humans, for example, tocilizumab did not protect mice from lethal neurotoxicity. In striking contrast, a IL-1 antagonist, anakinra, proved highly effective, either prophylactically or therapeutically, revealing IL-1 as a valuable target for global pharmacological intervention against life-threatening CAR-T cell toxicities. Selective responsiveness of neurotoxicity to anakinra is also supported by data in neonatal-onset multisystem inflammatory disease (NOMID)^{58,59}, an auto-inflammatory disease characterized by chronic aseptic meningitis, which is effectively reverted by the drug due to its CNS bioavailability.

At the current state of the art, it is debated whether CRS and neurotoxicity are restricted to CD19 CAR-T cells or, more in general, are to be expected with CAR-T cells specific for other tumor antigens. The inventors have recently developed a CD44v6-specific CAR-T cell strategy for treating AML and multiple myeloma, which express the antigen at high levels and are effectively targeted²¹. By using the inventors' human xenograft mouse model, the inventors here demonstrate that severe CRS and lethal neurotoxicity are likely common to all CAR-T cell antigens, provided that similarly effective in vivo tumor recognition is achieved. Interestingly, the inventors also found that in case of CD44v6 CAR-T cells, employing a BBz, rather than a 28z design, was detrimental in terms of toxicity. Differently from CD44v6.28z CAR-T cells, which rapidly ablated circulating monocytes, therefore protecting mice from CRS if given prophylactically, CD44v6.BBz CAR-T cells appeared to paradoxically induce proinflammatory monocyte licensing, resulting in 100% CRS mortality. While these findings might support the infusion of CD44v6.28z CAR-T cells soon after HSCT as a way to prevent toxicities, the observation of increased relapse rates due to prolonged monocyte aplasia warrants the implementation of a suicide gene in order to switch-off delayed unwanted effects²¹.

In summary, by using a newly developed xenotolerant mouse model, the inventors have demonstrated that monocyte-derived IL-1 and IL-6 are required for CRS and neurotoxicity by cell therapy, in particular CAR-T cells, and that targeted intervention against IL-1 may successfully overcome both toxicities, without interfering with antileukemia efficacy.

### BIBLIOGRAPHY

1. Sadelain, M. Curr Opin Immunol 41, 68-76 (2016).

2. Brentjens, R. J., et al. Blood 118, 4817-4828 (2011).

3. Savoldo, B., et al. J Clin Invest 121, 1822-1826 (2011).

4. Kochenderfer, J. N., et al. Blood 119, 2709-2720 (2012).

5. Porter, D. L., et al. N Engl J Med 365, 725-733 (2011).

6. Grupp, S. A., et al. N Engl J Med 368, 1509-1518 (2013).

7. Porter, D. L., et al. Sci Transl Med 7, 303ra139 (2015).

8. Turtle, C. J., et al. J Clin Oncol, JCO2017728519 (2017).

9. Brentjens, R. J., et al. Sci Transl Med 5, 177ra138 (2013).

10. Maude, S. L., et al. N Engl J Med 371, 1507-1517 (2014).

11. Lee, D. W., et al. Lancet 385, 517-528 (2015).

12. Turtle, C. J., et al. J Clin Invest 126, 2123-2138 (2016).

13. Kochenderfer, J. N., et al. J Clin Oncol 33, 540-549 (2015).

14. Turtle, C. J., et al. Sci Transl Med 8, 355ra116 (2016).

15. Schuster, S. J., et al. N Engl J Med 377, 2545-2554 (2017).

16. Neelapu, S. S., et al. N Engl J Med 377, 2531-2544 (2017).

17. Davila, M. L., et al. Sci Transl Med 6, 224ra225 (2014).

18. Teachey, D. T., et al. Cancer Discov 6, 664-679 (2016).

19. Lee, D. W., et al. Blood 124, 188-195 (2014).

20. Topp, M. S., et al. Lancet Oncol 16, 57-66 (2015).

21. Casucci, M., et al. Blood 122, 3461-3472 (2013).

22. Gill, S., et al. Blood 123, 2343-2354 (2014).

23. Sotillo, E., et al. Cancer Discov 5, 1282-1295 (2015).

24. Zhao, Z., et al. Cancer Cell 28, 415-428 (2015).

25. Long, A. H., et al. Nat Med 21, 581-590 (2015).

26. Sommermeyer, D., et al. Leukemia 30, 492-500 (2016).

27. Gomes-Silva, D., et al. Blood 130, 285-296 (2017).

28. Sabatino, M., et al. Blood 128, 519-528 (2016).

29. Bondanza, A., et al. Blood 107, 1828-1836 (2006).

30. Mastaglio, S., et al. Blood (2017).

31. Traggiai, E., et al. Science 304, 104-107 (2004).

32. Billerbeck, E., et al. Blood 117, 3076-3086 (2011).

33. Rongvaux, A., et al. Nat Biotechnol 32, 364-372 (2014).

34. Rongvaux, A., et al. Annu Rev Immunol 31, 635-674 (2013).

35. Kochenderfer, J. N., et al., Blood 116, 3875-3886 (2010).

36. Davila, M. L., Kloss, C. C., Gunset, G. & Sadelain, M. PLoS One 8, e61338 (2013).

37. Paszkiewicz, P. J., et al. J Clin Invest 126, 4262-4272 (2016).

38. Ghosh, A., et al. Nat Med 23, 242-249 (2017).

39. Gattinoni, L., Nat Med 23, 18-27 (2017).

40. Kaneko, S., et al. Blood 113, 1006-1015 (2009).

41. Bondanza, A., et al. Blood 117, 6469-6478 (2011).

42. Cieri, N., et al. Blood 121, 573-584 (2013).

43. Rongvaux, A., et al. Annu Rev Immunol 31, 635-674 (2013).

44. Shultz, L. D., et al. J Immunol 154, 180-191 (1995).

45. Takenaka, K., et al. Nat Immunol 8, 1313-1323 (2007).

46. Zheng, G. X., et al. Nat Commun 8, 14049 (2017).

47. Levine, J. H., et al. Cell 162, 184-197 (2015).

48. Villani, A. C., et al. Science 356(2017).

49. Wils, E. J., et al. J Immunol 187, 2974-2981 (2011).

50. Koo, G. C., Hasan, A. & O'Reilly, R. J. Expert Rev Vaccines 8, 113-120 (2009).

51. Strowig, T., et al. J Exp Med 206, 1423-1434 (2009).

52. Lan, P., et al., Blood 108, 487-492 (2006).

53. Bouma, G., et al. Eur J Immunol 35, 225-235 (2005).

54. Patel, A. A., et al. J Exp Med 214, 1913-1923 (2017).

55. Hunter, C. A. & Jones, S. A. Nat Immunol 16, 448-457 (2015).

56. Singh, N., et al. Cytotherapy 19, 867-880 (2017).

57. Gust, J., et al. Cancer Discov 7, 1404-1419 (2017).

58. Goldbach-Mansky, R., et al. N Engl J Med 355, 581-592 (2006).

59. Fox, E., et al. J Neuroimmunol 223, 138-140 (2010).

1. A method for the treatment and/or prevention of toxicity induced by a T cell therapy wherein the T cell expresses at least one recombinant receptor, comprising administering an IL-1 antagonist to a patient in need thereof.

2. The method according to claim 1 wherein:
(a) the administration of the IL-1 antagonist is:
at a time that is less than or no more than ten, seven, six, five, four or three days after initiation of the administration of the cell therapy; and/or
at a time at which the subject does not exhibit a sign or symptom of toxicity; and/or
(b) between the time of the initiation of the administration of the cell therapy and the time of the administration of the IL-1 antagonist, the subject has not exhibited toxicity; and/or
(c) the administration of the IL-1 antagonist is performed before or simultaneously to the T cell therapy.

3. The method according to claim 1, wherein the IL-1 antagonist is selected from the group consisting of: anakinra, rilonacept, canakinumab, gevokizumab, LY2189102, MABp1, MEDI-8968, CYT013, sIL-1RI, sIL-1RII, EBI-005, CMPX-1023, VX-765.

4. The method according claim 1, wherein the toxicity is selected from the group consisting of cytokine release syndrome, neurotoxicity, delayed toxicity.

5. The method according to claim 1, wherein the physical signs or symptoms associated with neurotoxicity, optionally severe neurotoxicity are selected from among confusion, delirium, expressive aphasia, obtundation, myoclonus, lethargy, altered mental status, convulsions, seizure-like activity, seizures (optionally as confirmed by electroencephalogram [EEG]), encephalopathy, dysphasia, tremor, choreoathetosis, symptoms that limit self-care, symptoms of peripheral motor neuropathy, symptoms of peripheral sensory neuropathy and combinations thereof; and/or the physical signs or symptoms associated with toxicity, optionally severe neurotoxicity, are associated with grade 3, grade 4 or grade 5 neurotoxicity; and/or the physical signs or symptoms associated with neurotoxicity, optionally severe neurotoxicity, manifest greater than or greater than about or about 5 days after cell therapy, 6 days after cell therapy or 7 days after T cell therapy.

6. The method according to claim 1 wherein the physical signs or symptoms associated with neurotoxicity, are selected from among acute inflammatory response and/or endothelial organ damage, fever, rigors, chills, hypotension, dyspnea, acute respiratory distress syndrome (ARDS), encephalopathy, ALT/AST elevation, renal failure, cardiac disorders, hypoxia, neurologic disturbances, and death, neurological complications such as delirium, seizure-like activity, confusion, word-finding difficulty, aphasia, and/or becoming obtunded, or fatigue, nausea, headache, seizure, tachycardia, myalgias, rash, acute vascular leak syndrome, liver function impairment, and renal failure and combinations thereof; and/or the physical signs or symptoms associated with toxicity manifest greater than or greater than about or about 5 days after cell therapy, 6 days after cell therapy or 7 days after cell therapy.

7. The method according to claim 1 wherein the T cell therapy is associated with or is capable of inducing toxicity, and wherein the T cell therapy optionally is adoptive T cell therapy and/or wherein the T cell therapy comprises administration of a dose of cells to treat a disease or condition in the subject.

8. The method according to claim 7, wherein the disease or condition is a cancer.

9. The method according to claim 1 wherein the dose of T cells comprises a number of cells between about 0.5×106 cells/kg body weight of the subject and 3×106 cells/kg, between about 0.75×106 cells/kg and 2.5×106 cells/kg or between about 1×106 cells/kg and 2×106 cells/kg.

10. The method according to claim 1 wherein the dose of T cells comprises a number of cells between about 1×105 cells/kg and 5×107 cells/kg, 2×105 cells/kg and 2×107 cells/kg, 2×105 cells/kg and 1×107 cells/kg, 2×105 cells/kg and 5×106 cells/kg, 2×105 cells/kg and 2×106 cells/kg or 2×105 cells/kg and 1×106 cells/kg.

11. The method according to claim 1 in combination with administering a further therapeutic agent.

12. The method according to claim 11 wherein the further therapeutic agent is a IL-6 antagonist or a chemotherapeutic agent, preferably the further therapeutic agent is selected from among tocilizumab, siltuximab, sarilumab, clazakizumab, olokizumab (CDP6038), elsilimomab, ALD518/BMS-945429, sirukumab (CNTO 136), CPSI-2634, ARGX-109, FE301, FMlOl, Hu-Mik-β-I, tofacitinib, ruxolitinib, CCX140-B, R0523444, BMS CCR2 22, INCB 3284 dimesylate, JNJ27141491 and RS 504393, adalimumab, certolizumab pegol, golimumab, lenalidomide, ibrutinib or acalabrutinib.

13. The method according to claim 1 wherein the recombinant receptor binds to, recognizes or targets an antigen associated with the disease or condition; and/or the recombinant receptor is a T cell receptor or a functional non-T cell receptor; and/or the recombinant receptor is a chimeric antigen receptor (CAR).

14. The method according to claim 13 wherein the CAR comprises an extracellular antigen-recognition domain that specifically binds to the antigen and an intracellular signaling domain comprising an IT AM, wherein optionally, the intracellular signaling domain comprises an intracellular domain of a CD3-zeta chain; and/or wherein the CAR further comprises a costimulatory signaling region, which optionally comprises a signaling domain of CD28 or 4-IBB.

15. The method according to claim 14 wherein the antigen is CD19 or CD 44v6.

16. The method according to claim 1 wherein the T cell is a CD4+ or CD8+ T cell.

17-20. (canceled)
